SEQUENCE LISTING (80 sequences)

SEQ ID NO: 1 - DNA, 12651 bp, Lactococcus lactis
  Eps gene cluster 33204, 33205, 33220, 33221, 33218, 33219, 33224, 33197, 33196, 33195, 33194, 33226, 33223, 33193 and 33192
atgaatgatttattttaccatcgtctaaaggaactagttgaatcaagtggtaaatctgcaaatcaaatagaaagggaattgggttaccctagaaattctttgaataattataagttgggaggagaaccctctgggacaagattaataggactatcagagtattttaatgtgtctccaaaatatctgatgggtataagtgatgagcctaatgacagttctgcaattaatctttttaaaactctaactcaagaagagaaaaaagaaatgtttataatttgtcaaaaatggctttttttagaatatcaaatagagttataacaataataaatttagggagtttttcttattaatatgatgaaaaaaggaatttttgtaattactatagtgatatctatagcattgataattggaggttttttatagttataattctaggataaataatctttcaaaagctgataaaggaaaagaagttgtaaaaaatagcagtgaaaaaaatcagatagaccttacctataaaaagtattataaaaatttaccaaaatcagttcaaaataaaatagatgatatttcatccaaaaataaagaagttactttaacttgtatttggcaatctgattcagttatttctgaacaatttcaacaaaacttacaaaaatattatggaaataagttttggaacatcaaaaatatcacctacaatggcgaaacaagtgaacaattattggctgaaaaagttcaaaatcaagtattggcgactaaccctgatgttgttttatatgaagctccactttttaatgataaccaatatagactactgggctagttacccagacaaaaattctgatgaaatgaaggggctgttttctgatgatggagtatatagaacattaaatgcttcggggaataaggtttggctagattatattactaaatattttacagcaaactaattaagttataaataacaattattaaatattggagaagaaatgcaggaaacacaggaacagacgattgatttaagagggatttttaaaattattcgcaaaaggttaggtttaatattatttagtgctttaatagtcacaatattagggagcatctacacattttttatagcctccccagtttacacagcctcaactcaacttgtcgttaaactaccaaattcggataattcagcagcctacgctggagaagtgaccgggaatattcaaatggcgaacacaattaaccaagttattgttagtccagtcattttagataaagttcaaagtaatttaaatctatctgatgactctttccaaaaacaagttacagcagcaaatcaaacaaattcacaagttattatgcttactgttaaatattctaatccttacattgcaaaaaagattgcagacgagactgctaaaatttttagttcagatgcagcaaaactattgaatgttactaacgttaatattctatccaaagcaaaagctcaaacaacaccaattagtcctaaacctaaattgtatttagcgatatctgttatagccggactagttttaggtttagccattgctttattgaaggaattgtttgataacaaaattaataaagaagaagatattgaagctctggggctcacggttcttggtgtaacaagctatgatcaaatgagtgattttaataagaatacaaataaaaatggcacgcaatcgggaactaagtcaagtccgcctagcgaccatgaagtaaatagatcatcaaaaaggaataaaagataggagttcaggatggctaaaaataaaagaagcatagacaataatcattatattattaccagtgtcaatcctcaatcacctatttccgaacaatatcgtacgattcgtacgaccattgattttaaaatggcggatcaaggaattaaaagttttctagtaacatcttcagaaacagatgaaggtaaaacaaccgtaagtgctaatatagctgttgcttttgcacaacaaggtaaaaaagtacttttaattgatggcgatcttcgtaaaccgactgttaacattacttttaaagtacaaaatagagtaggattaaccaatattttaatgcatcaatcttcgattgaagatgccatacaagggacaagactttctgaaaatcttacaataattacctctggtccaattccacctaatccatcggaattattagcatctagtgcaatgaagaatttgattgactctgtgtccgatttctttgatgttgttttgattgatattccacctctctctgcagttactgatgctcaaattttgagtagttatgtaggaggagtggttcttgttgtacgtgcctatgaaacaaaaaaagagagtttagcaaaaacaaaaaaaaagctggaacaagttaatgcaaatatattaggagttgttttgcatggggtagactcttctgactcaccgtcgtattactactacggagtagagtaattggaataaattttaatcaaataaaagacagaaatttgtagaagaggagagcaaatgattgatattcattgccatattttaccgggtatagatgatggagctaaaacttctggagatactttgacaatgctgaaatcagcaattgatgaagggataacaaccatcaccgctactcctcatcataatcctcaatttaataatgaatcaccacttattttaaaaaaagttaaggaagttcaaaatatcattgacgagcatcaattaccaattgaagttttgcctggacaagaggttagaatatatggtgatttattaaaagaattttctgaaggaaagttactgaaagcagcgggcacttcaagttatatattgattgaatttccatcaaatcatgtgccagcttatgctaaagaacttttttataatattaaattggagggccttcaacctattttggtccaccctgagcgtaatagtggaatcattgagaaccctgatatattatttgattttattgaacaaggagtactaagtcagataacagcttcaagtgtcactggtcattttggtaaaaaaatacaaaagctatcatttaaaatgatagaaaaccatcttacgcattttgttgcatcagatgcgcataatgtgacgtcacgtgcatttaagatgaaggaagcatttgaaattattgaagatagttatggttctggtgtatcacgaatgttacaaaataatgcagactcggtgattttgaacgaaagtttttatcaagaagaaccaataaaaattaaaacaaagaaatttttgggattattttaaaaggattaaatggagtaaataatggaagtttttgaggcatcatctgaactggaagagcctaagttagtagaattaaaaaaattttctcgcagagagataattataaaaagagggattgatattttagggggattagcgggttcaggtttatttcttatcgcggctgcattgctttatgtcccttacaaaatgagctcaaaaaaagatcaagggccaatgttctataaacaaaaacggtatggaaaaaatggtaaaattttttatattttgaaatttagaacaatgataattaatgctgagcagtatttagagctacatccagaagttaaagccgcctatcatgccaatggcaataaactagaaagtgatccccgtgtaacgaagattgggtcatttattagacaacactcaattgatgaattaccacaatttatcaatgtccttaaaggagatatgtcattagttggtccaagaccaattttgctttttgaagcgaaagaatatggggagcgcctctcttacttactgatatgcaaacctggaattactggttattggacaacacatggtcgaagtaaagttctttttcctcaacgagcagatttagagctctattatctccagtaccatagtacaaaaaatgatataaaacttattatgcttacaataaaacaaattctacatggatcggatgcttattaaagtaacattatgaaaaaaaaaacaactaaaatttgcatgatttcttcttctgggggtcatttaaaagagcttaatgaattgatagagatttcagagcagtatgaaacgtttcaaattactgaaaaagataaattttctaatatcaagattggaactaggcaatactatgtgaataaaattgatagagatgaaaaaaattttttatttcatttttttattctttttttgaaaatatttcaaatatttgctgtagagaagcctaaagttatagtaaccactggtgccttagtagcttatccagcatgtctaataggaaaattaatgagagctaaagttatttttatagagtcttatgctcgaacagaaacattatcattaacaggaaaattagtttataggttatctgatttatttattgttcaatggccagatctttcaaaaaaatattctaaagctaaatactatggggaattattctgatgatattaataatattagggactcaaaaatttcaattcaaccgacttataaaaaaagttgataaattaatagaagatgatcaaatcaaagattctgtaatagctcaaatcggatattctaattacaaacctataaattataaattttcagatttttttgatcaatcggaatttgattcattaataaataaatcagatataataataactcatggaggagtaggtgggatagtttcttccttaaaaaagaataaaaaaatcatagtagttccgcgtttaaagaaatacagagaacatattgatgatcatcaattagagatagcaagggcgtttcaaagaaaaaatctagttattttaaacgagaatctaaatgaactatgtaatgatatatctaaaattgaatcattcgagccaatacactatgtcaaagataataaaaaaattatatgtgaaataaaaaaatttatatcgaaagttaaatgatatttttatacaaaattatcttatgatgagaaaggactttttaaaagataaaaaatgataaaattgagcattataattccaatttataacgtggaaaaatatttaagtaaatgtttaaattctattttagaacaaacttataaagaaatagaaataatattagtaaatgatggtagtactgataactcaaaagatatagctgtaagctattgtgaaagatttcctaatgtttttaaatattttgagaaagataacggaggcctctcttcagccagaaattttggacttgaaaaaatttctggtgattttgtaggcttcttagactcagatgactatatagataacgatttatatgaaattatgattaattcattggatagttcaataaaaattgtggaatgtgattttatatgggaatacgaaaatggaaaaagtgtccttgataaaacatctgaatataattctatcaaagacttaatggttaacggtagagttgttgcttggaataaaatatataatgttgaatggttagaaaaaataaacataaagtttaaagaaggtctattgtatgaagatttaaattttttcttcaaaattgttcctcacttgactagtatttcagaagtatcaacagttaaaaatagttttgttcactatgtccagcataaaggtacaataacttcagataattctcttaatatcttggatatcataaaatcttacgaagatgtctttcattattataacgaaaaacagattaatgatttatattttgatgagctagaatataaattttctaggaacttaatgggggcatttttaaaaagagcaattaagattaaagataaaagacaacgtaaaataattttagatgaattttggaataatgttttatcttactatccgaattggaaaaaaaataaatatataaaaaaactatcaaaacagaatatacttttattttttattaataaatatacatataaattattttatttattataaaaaaaatttaatattagagtatttgtattagttgcaatgaaaatatcgaaagtagaataaatgatttatgtagaaataaggggaaacttaggtaatcaattatttatctatgccaccgcaaaaaaaattcaaaagttaaccggacaaaaaattcaattaaatacaacaactttaaataaatactttccaaattacaagtttggcctttcagaatttataatggaggatcctgattgttttattgaatcctataaaaaattaccctggttcacaaacgagtatctcttacctattaaaatttttaaaaaaatattgaataaaacacccaaaattaataaaatcctttcagatttttttttcaaagcttttgaaaaaaaaggatattttatttggcgaggagagacttttaaaaagttttctttaggaaatcataaaaattactatttatcaggtttttggcaatcggaagaatatttttatgatataagggatgaattattagaaatcatcactcctataaattcaataagagagtgtaactttgaacttctcaatttaataaggaattcagaatcaatttgtgtttcaatacgccgaggagattatgtagataatcctaaaatatcagctatttataacgtatgtgatataaattattttatagaatctgtaaatgaaataaagaaaaatgttgtgaatgttaaagttatctgtttttcagatgatgttgaatgggtcaaaaaaaatataaaattcgactgtgaaacacattatgaaacttatggtaattctttatctgaaaaagttcaacttatgtcttcttgtaaacattttgttttatctaatagttcttttagttggtggacagaatttttatctatacgaggtgggattactatagcccccaaaaattggtatgcagatgaacgtgaagctgatatctatagaaaaaattggatttacttagaagataagacagaggaagagtaatgggatttctatttttaactataatacttattttgtgggggtatagttttaccaatataaaaataagcccttttagtattttattcatgagtttagggatcttttactctcaatttacttcaataaatattgacttaataataaaagtactttttttgataacttccataatttatcttattaaagataaatattcaaaaaaatacgttttttctttattattaattgcagtattaattttaattgagtcaactagtccctctaaatttaatcaatattatggttttattgatgctttgacatcatttgcaaccttctcaacaggcatacttctattttccataaaatttagtttacaagaacgcagaagtattttaaaatcaatttcatatttgccaatcttttcagtgttaattggaatccctctaacttttggtggttttatatctatgacagctagaggaggaattgccctttcaggagcagctttagaaacaaatttatcttttttttcagttctaagccttgtttcattagatattttatatcaggacactcgttctaataaatatcaaattttaaaaattattaactttatattgctatgttgtactttaacacgaggcggtattatttctggaattatcattattttaccaagtttactatttcttttaaaaaaaggatttaaaggagtaagacaatttatttttttgattattactatttttgggagtatttatccgcttattttattgtggaaaagtattagtgagaggactttcagtgcagatggtattaatacttcaggtcgatatacggcctgggactatattgtgaatttgacaacaaacaaatctcagggaatgggattgggaagtttaaagacattaactgaggatattaatttacgtgcctttactgctgctcataatacatatattcaattttattatgaaactggttatttgggagtaacactattatctattttatttattttaatattaataataatcctaaaattgactaattatagaaaaaaaatcatttacttaacattcatttcatttttagtatatagttatacagataattgtattgttaataatagatactggtatttgtttatgtttattataggatgttttaaatattttgacagaaaggaagaaaatgcgctactttaaaatattatttgagattattcaactattggtagctagtattttatgtagattatataaaaatccaaatgatatctggctaataaatgaaaaacctgatgaagctagagataatggttatgctttttatcaatatttaagaaagaatttccccgatattaaagtttattatgtaatcagtaaagagtctactgatatttataagtttgataatgaaactaacattgtattttataagagttttttacattttattttatatatcaaatctaaagttttaattagttctcaaacattgccctatccatctagcagaaaattatgtgaagcgctaatgtaccttaatttgaataaaccaaagaggatttggttacaacatggagttactaaagataaactcccatatgagaatatggcaagggaaatttttaagtatgatttaataacctgtgtttcattaaaagaggctaattttataatgaaagaatatggatataatgaagatcaggtgaaggctcttggatttgcaagatatgataatttgccaattggaaataataacacatttgatatacttataatgcctactttccgtaagggttacgagattaaaaattttagtctcccaacagatagtgaaactaaacattttgaggaaagtgtattctttaaaacatatgttgatttattgaattctgaagagctagacgagtatttagaaaagtctggtaaaaaagcaattttttatttacactatgcttttcaaccatatgcaaaatctttttctaaacgactaatgtcttcaaatgttatcattgctgaaagaacagaatatgatgttcaaaaactattaattaattgtgaattgctaattacagattattcaagcgttttttttgatttttcatatatgaaaaaacctgaaatatttttccattttgatgagaaagaatatagaagtaatcattatagggagggatattttgattataaaacagatggatttggtccagtagttaattctaaagaagaattactaactgaaatcaaagagtttattgataacccatctctgttaatggaatttaataagcgagctaataatttcttcaaatatactgataacaataattgccaacgtattttaaaagaaatttggagaattaatgaaactaattaagaattatttaatgacaagctcttatcaattgttaattattatcttaccaataataacaacaccatatatatcgagagtacttagtccagaagggattggactttattcatatacttatactattacacagtatttcgtattatttgctactcttggtactgttacgtatggtagcagagagatagcatattatcagtcaaataaacaaaagagaagtgaaattttttggggaattaccttccttagctgggctactggtgctatatcacttttaatattttatatatttattttttttaatggcaaatatagtgttttatttttttggcaaagctttttgatttttggagttatttttgatattaattggtatttcacaggaatggaaaagttcaaggttattatttcacgtaacttttgtataaaaattattagtttattgtgtatttttgtctttgtaaaatctgagaaagatttaagtttatatatagttatactaggattgagcaatataataggtaatatattagtttggccatatttgagaaaagaggtttataaacctaatttttctaagttatcattcaaaaaacatttgggaagtacatggatattttttttgccacaaacttctgttactttaaactcattaataaaccaaaatatgattgcatattttgactcaataacaagcttaggatactttacacaaacaaataagtttactgtgattgcgatttcaatagttatttcaattgggactgttatgttgcctagaatgtccaatttagttgcgcgcaaagagtattcaaagtttacagactatgttactaagagtattaatataagctcaggaatttctatagcaataatgtttggtttaatggctatagcacctaagtttacaactttttttttaggagctcaatataaatttgttattcatttgctagttttatcatcaccgatagtggttttagtaacctggagtaatgttcttggtcaacaatatttaatacctttaaataggatgaaaatatttacaaaatctctaatttgtggaaacttagtaaatgtttctctaaacttgattttgttacccaaaatgggagtagaaatttcaataataaatcagttaattaatgaaattattattgtaggtattcaatttatatcagttagaaaagagttaaaaataaatataatattaggagatctaataaaatatttttttgcgggtataattatgtttattgccgttttatatctgaatttacaattaccgatgactatcttcacactacttatagagattggtattggagttcttatatattcaatgctagttatttctcttaaaactggattatataaagaattgaaaaagattattaaaattcgttagcttaaaatctatcacctttcatttgagtagtaagaaatacaaagctttattataaaatttatcatttttaagactatcataaaagaagaaggatgacatggaacgaaaaaagaagaaaaaaaaaatatatataattattctaatattattaatgtttatcactattgtttgttttgggggatatgctacacgagagttaattactcccactgaaaaaacaataccaaatgtctcggatcaacctaaaaaaacttcggcctctaacggttatgtagagcaaaaaggggaagaagctgctgtgggtagtatagcacttgtagacgatgctggtgtaccagaatgggttaaagttccctcaaaggtaaatctagataaatttactgatttatctacgaataatatcactatttatcgaattaataatccggaagtcttaaaaacagttaccaatcgtacagatcaacggatgaaaatgtcagaagttatagctaagtatcctaatgctttgattatgaatgcttccgcttttgatatgcagacaggacaagtagctggatttcaaattaataatggaaagttgattcaagactggagtccaggtacaacgactcaatatgcttttgttattaacaaagatggttcgtgcaaaatttatgattcaagtacacctgctttaactattattaaaaatggagggcaacaagcctatgattttggtactgcgattatccgtgatggtaaaattcaaccaagtgatggctcagtagattggaagattcatatttttattgcgaatgataaagataataatctctatgctattttgagtgatacaaatgcaggttatgataatataataaaatcagtatcaaatttgaagctccaaaatatgttattacttgatagtggtggctcaagtcaactatctgtcaatggtaaaacgattgttgctagtcaagatgatcgagccgtaccggattatattgtgatgaaataaaaataaaagaacctcatggttcttttattttagagatttttcaaaaagggttttgactgagtctaattctgtttgagaaacgaccttagctccattttcatctgttgtatgtagattgagcttgctggtattctttagagccttactgtagctcataacgatcgttttagcatcattgaaacttatattggttttaacagtgtttgaaaaagcatagagaatttctcgatagtaattgaaactttcaagttttttcatatgagcaattttttgaatatttccgtagagttccattgagacattttgaatccgagtgattgaagcatccaaatcagtatcgtcaatttgtgtcatataggcttggacttgatcagcagtttgtaaattaacagttccttgtttaaactcataaccttcagccttgaatgcctttggattttgcatggtgattccaccagtggcctgtacaagtgatcccattttattaacatcgatctgaattactttgttaatggacgcattcaataggtctttaaccatctggaaaattccatcatctccattcgtattgtaaacttcagtgattgttttttgattaggcattgtcgcaaaaactgggaagttcatgaaagtagtttgatttgtctttacattcgttgaagctaaaacagtagcataagctgaatttttagaattatttttaccagttgcaatgataagtgtggtgaatgttttagctttttttaagtcaatacttgttgttttagggaaattttcatatgatgttgaaaaggttgattcaacatttctataagctacgtaggctatagaagcaacagaaataattactaatacaaaaataattgaaataacttttagtactgtgtattttttcttacgataatgacgcctctttttttgattcat

SEQ ID NO: 2 - DNA, 12651 bp, Lactococcus lactis
  Eps gene cluster of 33200, 33201, 33202 and 33203
atgaatgatttattttaccatcgtctaaaggaactagttgaatcaagtggtaaatctgcaaatcaaatagaaagggaattgggttaccctagaaattctttgaataattataagttgggaggagaaccctctgggacaagattaataggactatcagagtattttaatgtgtctccaaaatatctgatgggtataagtgatgagcctaatgacagttctgcaattaatctttttaaaactctaactcaagaagagaaaaaagaaatgtttataatttgtcaaaaatggctttttttagaatatcaaatagagttataacaataataaatttagggagtttttcttattaatatgatgaaaaaaggaatttttgtaattactatagtgatatctatagcattgataattggaggttttttatagttataattctaggataaataatctttcaaaagctgataaaggaaaagaagttgtaaaaaatagcagtgaaaaaaatcagatagaccttacctataaaaagtattataaaaatttaccaaaatcagttcaaaataaaatagatgatatttcatccaaaaataaagaagttactttaacttgtatttggcaatctgattcagttatttctgaacaatttcaacaaaacttacaaaaatattatggaaataagttttggaacatcaaaaatatcacctacaatggcgaaacaagtgaacaattattggctgaaaaagttcaaaatcaagtattggcgactaaccctgatgttgttttatatgaagctccactttttaatgataaccaatatagactactgggctagttacccagacaaaaattctgatgaaatgaaggggctgttttctgatgatggagtatatagaacattaaatgcttcggggaataaggtttggctagattatattactaaatattttacagcaaactaattaagttataaataacaattattaaatattggagaagaaatgcaggaaacacaggaacagacgattgatttaagagggatttttaaaattattcgcaaaaggttaggtttaatattatttagtgctttaatagtcacaatattagggagcatctacacattttttatagcctccccagtttacacagcctcaactcaacttgtcgttaaactaccaaattcggataattcagcagcctacgctgtagaagtgaccgggaatattcaaatggcgaacacaattaaccaagttattgttagtccagtcattttagataaagttcaaagtaatttaaatctatctgatgactctttccaaaaacaagttacagcagcaaatcaaacaaattcacaagttattatgcttactgttaaatattctaatccttacattgcaaaaaagattgcagacgagactgctaaaatttttagttcagatgcagcaaaactattgaatgttactaacgttaatattctatccaaagcaaaagctcaaacaacaccaattagtcctaaacctaaattgtatttagcgatatctgttatagccggactagttttaggtttagccattgctttattgaaggaattgtttgataacaaaattaataaagaagaagatattgaagctctggggctcacggttcttggtgtaacaagctatgatcaaatgagtgattttaataagaatacaaataaaaatggcacgcaatcgggaactaagtcaagtccgcctagcgaccatgaagtaaatagatcatcaaaaaggaataaaagataggagttcaggatggctaaaaataaaagaagcatagacaataatcattatattattaccagtgtcaatcctcaatcacctatttccgaacaatatcgtacgattcgtacgaccattgattttaaaatggcggatcaaggaattaaaagttttctagtaacatcttcagaaacagatgaaggtaaaacaaccgtaagtgctaatatagctgttgcttttgcacaacaaggtaaaaaagtacttttaattgatggcgatcttcgtaaaccgactgttaacattacttttaaagtacaaaatagagtaggattaaccaatattttaatgcatcaatcttcgattgaagatgccatacaagggacaagactttctgaaaatcttacaataattacctctggtccaattccacctaatccatcggaattattagcatctagtgcaatgaagaatttgattgactctgtgtccgatttctttgatgttgttttgattgatattccacctctctctgcagttactgatgctcaaattttgagtagttatgtaggaggagtggttcttgttgtacgtgcctatgaaacaaaaaaagagagtttagcaaaaacaaaaaaaaagctggaacaagttaatgcaaatatattaggagttgttttgcatggggtagactcttctgactcaccgtcgtattactactacggagtagagtaattggaataaattttaatcaaataaaagacagaaatttgtagaagaggagagcaaatgattgatattcattgccatattttaccgggtatagatgatggagctaaaacttctggagatactttgacaatgctgaaatcagcaattgatgaagggataacaaccatcaccgctactcctcatcataatcctcaatttaataatgaatcaccacttattttaaaaaaagttaaggaagttcaaaatatcattgacgagcatcaattaccaattgaagttttgcctggacaagaggttagaatatatggtgatttattaaaagaattttctgaaggaaagttactgaaagcagcgggcacttcaagttatatattgattgaatttccatcaaatcatgtgccagcttatgctaaagaacttttttataatattaaattggagggccttcaacctattttggtccaccctgagcgtaatagtggaatcattgagaaccctgatatattatttgattttattgaacaaggagtactaagtcagataacagcttcaagtgtcactggtcattttggtaaaaaaatacaaaagctatcatttaaaatgatagaaaaccatcttacgcattttgttgcatcagatgcgcataatgtgacgtcacgtgcatttaagatgaaggaagcatttgaaattattgaagatagttatggttctggtgtatcacgaatgttacaaaataatgcagactcggtgattttgaacgaaagtttttatcaagaagaaccaataaaaattaaaacaaagaaatttttgggattattttaaaaggattaaatggagtaaataatggaagtttttgaggcatcatctgaactggaagagcctaagttagtagaattaaaaaaattttctcgcagagagataattataaaaagagggattgatattttagggggattagcgggttcaggtttatttcttatcgcggctgcattgctttatgtcccttacaaaatgagctcaaaaaaagatcaagggccaatgttctataaacaaaaacggtatggaaaaaatggtaaaattttttatattttgaaatttagaacaatgataattaatgctgagcagtatttagagctacatccagaagttaaagccgcctatcatgccaatggcaataaactagaaagtgatccccgtgtaacgaagattgggtcatttattagacaacactcaattgatgaattaccacaatttatcaatgtccttaaaggagatatgtcattagttggtccaagaccaattttgctttttgaagcgaaagaatatggggagcgcctctcttacttactgatatgcaaacctggaattactggttattggacaacacatggtcgaagtaaagttctttttcctcaacgagcagatttagagctctattatctccagtaccatagtacaaaaaatgatataaaacttattatgcttacaataaaacaaattctacatggatcggatgcttattaaagtaacattatgaaaaaaaaaacaactaaaatttgcatgatttcttcttctgggggtcatttaaaagagcttaatgaattgatagagatttcagagcagtatgaaacgtttcaaattactgaaaaagataaattttctaatatcaagattggaactaggcaatactatgtgaataaaattgatagagatgaaaaaaattttttatttcatttttttattctttttttgaaaatatttcaaatatttgctgtagagaagcctaaagttatagtaaccactggtgccttagtagcttatccagcatgtctaataggaaaattaatgagagctaaagttatttttatagagtcttatgctcgaacagaaacattatcattaacaggaaaattagtttataggttatctgatttatttattgttcaatggccagatctttcaaaaaaatattctaaagctaaatactatggggaattattctgatgatattaataatattagggactcaaaaatttcaattcaaccgacttataaaaaaagttgataaattaatagaagatgatcaaatcaaagattctgtaatagctcaaatcggatattctaattacaaacctataaattataaattttcagatttttttgatcaatcggaatttgattcattaataaataaatcagatataataataactcatggaggagtaggtgggatagtttcttccttaaaaaagaataaaaaaatcatagtagttccgcgtttaaagaaatacagagaacatattgatgatcatcaattagagatagcaagggcgtttcaaagaaaaaatctagttattttaaacgagaatctaaatgaactatgtaatgatatatctaaaattgaatcattcgagccaatacactatgtcaaagataataaaaaaattatatgtgaaataaaaaaatttatatcgaaagttaaatgatatttttatacaaaattatcttatgatgagaaaggactttttaaaagataaaaaatgataaaattgagcattataattccaatttataacgtggaaaaatatttaagtaaatgtttaaattctattttagaacaaacttataaagaaatagaaataatattagtaaatgatggtagtactgataactcaaaagatatagctgtaagctattgtgaaagatttcctaatgtttttaaatattttgagaaagataacggaggcctctcttcagccagaaattttggacttgaaaaaatttctggtgattttgtaggcttcttagactcagatgactatatagataacgatttatatgaaattatgattaattcattggatagttcaataaaaattgtggaatgtgattttatatgggaatacgaaaatggaaaaagtgtccttgataaaacatctgaatataattctatcaaagacttaatggttaacggtagagttgttgcttggaataaaatatataatgttgaatggttagaaaaaataaacataaagtttaaagaaggtctattgtatgaagatttaaattttttcttcaaaattgttcctcacttgactagtatttcagaagtatcaacagttaaaaatagttttgttcactatgtccagcataaaggtacaataacttcagataattctcttaatatcttggatatcataaaatcttacgaagatgtctttcattattataacgaaaaacagattaatgatttatattttgatgagctagaatataaattttctaggaacttaatgggggcatttttaaaaagagcaattaagattaaagataaaagacaacgtaaaataattttagatgaattttggaataatgttttatcttactatccgaattggaaaaaaaataaatatataaaaaaactatcaaaacagaatatacttttattttttattaataaatatacatataaattattttatttattataaaaaaaatttaatattagagtatttgtattagttgcaatgaaaatatcgaaagtagaataaatgatttatgtagaaataaggggaaacttaggtaatcaattatttatctatgccaccgcaaaaaaaattcaaaagttaaccggacaaaaaattcaattaaatacaacaactttaaataaatactttccaaattacaagtttggcctttcagaatttataatggaggatcctgattgttttattgaatcctataaaaaattaccctggttcacaaacgagtatctcttacctattaaaatttttaaaaaaatattgaataaaacacccaaaattaataaaatcctttcagatttttttttcaaagcttttgaaaaaaaaggatattttatttggcgaggagagacttttaaaaagttttctttaggaaatcataaaaattactatttatcaggtttttggcaatcggaagaatatttttatgatataagggatgaattattagaaatcatcactcctataaattcaataagagagtgtaactttgaacttctcaatttaataaggaattcagaatcaatttgtgtttcaatacgccgaggagattatgtagataatcctaaaatatcagctatttataacgtatgtgatataaattattttatagaatctgtaaatgaaataaagaaaaatgttgtgaatgttaaagttatctgtttttcagatgatgttgaatgggtcaaaaaaaatataaaattcgactgtgaaacacattatgaaacttatggtaattctttatctgaaaaagttcaacttatgtcttcttgtaaacattttgttttatctaatagttcttttagttggtggacagaatttttatctatacgaggtgggattactatagcccccaaaaattggtatgcagatgaacgtgaagctgatatctatagaaaaaattggatttacttagaagataagacagaggaagagtaatgggatttctatttttaactataatacttattttgtgggggtatagttttaccaatataaaaataagcccttttagtattttattcatgagtttagggatcttttactctcaatttacttcaataaatattgacttaataataaaagtactttttttgataacttccataatttatcttattaaagataaatattcaaaaaaatacgttttttctttattattaattgcagtattaattttaattgagtcaactagtccctctaaatttaatcaatattatggttttattgatgctttgacatcatttgcaaccttctcaacaggcatacttctattttccataaaatttagtttacaagaacgcagaagtattttaaaatcaatttcatatttgccaatcttttcagtgttaattggaatccctctaacttttggtggttttatatctatgacagctagaggaggaattgccctttcaggagcagctttagaaacaaatttatcttttttttcagttctaagccttgtttcattagatattttatatcaggacactcgttctaataaatatcaaattttaaaaattattaactttatattgctatgttgtactttaacacgaggcggtattatttctggaattatcattattttaccaagtttactatttcttttaaaaaaaggatttaaaggagtaagacaatttatttttttgattattactatttttgggagtatttatccgcttattttattgtggaaaagtattagtgagaggactttcagtgcagatggtattaatacttcaggtcgatatacggcctgggactatattgtgaatttgacaacaaacaaatctcagggaatgggattgggaagtttaaagacattaactgaggatattaatttacgtgcctttactgctgctcataatacatatattcaattttattatgaaactggttatttgggagtaacactattatctattttatttattttaatattaataataatcctaaaattgactaattatagaaaaaaaatcatttacttaacattcatttcatttttagtatatagttatacagataattgtattgttaataatagatactggtatttgtttatgtttattataggatgttttaaatattttgacagaaaggaagaaaatgcgctactttaaaatattatttgagattattcaactattggtagctagtattttatgtagattatataaaaatccaaatgatatctggctaataaatgaaaaacctgatgaagctagagataatggttatgctttttatcaatatttaagaaagaatttccccgatattaaagtttattatgtaatcagtaaagagtctactgatatttataagtttgataatgaaactaacattgtattttataagagttttttacattttattttatatatcaaatctaaagttttaattagttctcaaacattgccctatccatctagcagaaaattatgtgaagcgctaatgtaccttaatttgaataaaccaaagaggatttggttacaacatggagttactaaagataaactcccatatgagaatatggcaagggaaatttttaagtatgatttaataacctgtgtttcattaaaagaggctaattttataatgaaagaatatggatataatgaagatcaggtgaaggctcttggatttgcaagatatgataatttgccaattggaaataataacacatttgatatacttataatgcctactttccgtaagggttacgagattaaaaattttagtctcccaacagatagtgaaactaaacattttgaggaaagtgtattctttaaaacatatgttgatttattgaattctgaagagctagacgagtatttagaaaagtctggtaaaaaagcaattttttatttacactatgcttttcaaccatatgcaaaatctttttctaaacgactaatgtcttcaaatgttatcattgctgaaagaacagaatatgatgttcaaaaactattaattaattgtgaattgctaattacagattattcaagcgttttttttgatttttcatatatgaaaaaacctgaaatatttttccattttgatgagaaagaatatagaagtaatcattatagggagggatattttgattataaaacagatggatttggtccagtagttaattctaaagaagaattactaactgaaatcaaagagtttattgataacccatctctgttaatggaatttaataagcgagctaataatttcttcaaatatactgataacaataattgccaacgtattttaaaagaaatttggagaattaatgaaactaattaagaattatttaatgacaagctcttatcaattgttaattattatcttaccaataataacaacaccatatatatcgagagtacttagtccagaagggattggactttattcatatacttatactattacacagtatttcgtattatttgctactcttggtactgttacgtatggtagcagagagatagcatattatcagtcaaataaacaaaagagaagtgaaattttttggggaattaccttccttagctgggctactggtgctatatcacttttaatattttatatatttattttttttaatggcaaatatagtgttttatttttttggcaaagctttttgatttttggagttatttttgatattaattggtatttcacaggaatggaaaagttcaaggttattatttcacgtaacttttgtataaaaattattagtttattgtgtatttttgtctttgtaaaatctgagaaagatttaagtttatatatagttatactaggattgagcaatataataggtaatatattagtttggccatatttgagaaaagaggtttataaacctaatttttctaagttatcattcaaaaaacatttgggaagtacatggatattttttttgccacaaacttctgttactttaaactcattaataaaccaaaatatgattgcatattttgactcaataacaagcttaggatactttacacaaacaaataagtttactgtgattgcgatttcaatagttatttcaattgggactgttatgttgcctagaatgtccaatttagttgcgcgcaaagagtattcaaagtttacagactatgttactaagagtattaatataagctcaggaatttctatagcaataatgtttggtttaatggctatagcacctaagtttacaactttttttttaggagctcaatataaatttgttattcatttgctagttttatcatcaccgatagtggttttagtaacctggagtaatgttcttggtcaacaatatttaatacctttaaataggatgaaaatatttacaaaatctctaatttgtggaaacttagtaaatgtttctctaaacttgattttgttacccaaaatgggagtagaaatttcaataataaatcagttaattaatgaaattattattgtaggtattcaatttatatcagttagaaaagagttaaaaataaatataatattaggagatctaataaaatatttttttgcgggtataattatgtttattgccgttttatatctgaatttacaattaccgatgactatcttcacactacttatagagattggtattggagttcttatatattcaatgctagttatttctcttaaaactggattatataaagaattgaaaaagattattaaaattcgttagcttaaaatctatcacctttcatttgagtagtaagaaatacaaagctttattataaaatttatcatttttaagactatcataaaagaagaaggatgacatggaacgaaaaaagaagaaaaaaaaaatatatataattattctaatattattaatgtttatcactattgtttgttttgggggatatgctacacgagagttaattactcccactgaaaaaacaataccaaatgtctcggatcaacctaaaaaaacttcggcctctaacggttatgtagagcaaaaaggggaagaagctgctgtgggtagtatagcacttgtagacgatgctggtgtaccagaatgggttaaagttccctcaaaggtaaatctagataaatttactgatttatctacgaataatatcactatttatcgaattaataatccggaagtcttaaaaacagttaccaatcgtacagatcaacggatgaaaatgtcagaagttatagctaagtatcctaatgctttgattatgaatgcttccgcttttgatatgcagacaggacaagtagctggatttcaaattaataatggaaagttgattcaagactggagtccaggtacaacgactcaatatgcttttgttattaacaaagatggttcgtgcaaaatttatgattcaagtacacctgctttaactattattaaaaatggagggcaacaagcctatgattttggtactgcgattatccgtgatggtaaaattcaaccaagtgatggctcagtagattggaagattcatatttttattgcgaatgataaagataataatctctatgctattttgagtgatacaaatgcaggttatgataatataataaaatcagtatcaaatttgaagctccaaaatatgttattacttgatagtggtggctcaagtcaactatctgtcaatggtaaaacgattgttgctagtcaagatgatcgagccgtaccggattatattgtgatgaaataaaaataaaagaacctcatggttcttttattttagagatttttcaaaaagggttttgactgagtctaattctgtttgagaaacgaccttagctccattttcatctgttgtatgtagattgagcttgctggtattctttagagccttactgtagctcataacgatcgttttagcatcattgaaacttatattggttttaacagtgtttgaaaaagcatagagaatttctcgatagtaattgaaactttcaagttttttcatatgagcaattttttgaatatttccgtagagttccattgagacattttgaatccgagtgattgaagcatccaaatcagtatcgtcaatttgtgtcatataggcttggacttgatcagcagtttgtaaattaacagttccttgtttaaactcataaccttcagccttgaatgcctttggattttgcatggtgattccaccagtggcctgtacaagtgatcccattttattaacatcgatctgaattactttgttaatggacgcattcaataggtctttaaccatctggaaaattccatcatctccattcgtattgtaaacttcagtgattgttttttgattaggcattgtcgcaaaaactgggaagttcatgaaagtagtttgatttgtctttacattcgttgaagctaaaacagtagcataagctgaatttttagaattatttttaccagttgcaatgataagtgtggtgaatgttttagctttttttaagtcaatacttgttgttttagggaaattttcatatgatgttgaaaaggttgattcaacatttctataagctacgtaggctatagaagcaacagaaataattactaatacaaaaataattgaaataacttttagtactgtgtattttttcttacgataatgacgcctctttttttgattcat

SEQ ID NO: 3 - DNA, 12651 bp, Lactococcus lactis
  Eps gene cluster of 33222
atgaatgatttattttaccatcgtctaaaggaactagttgaatcaagtggtaaatctgcaaatcaaatagaaagggaattgggttaccctagaaattctttgaataattataagttgggaggagaaccctctgggacaagattaataggactatcagagtattttaatgtgtctccaaaatatctgatgggtataagtgatgagcctaatgacagttctgcaattaatctttttaaaactctaactcaagaagagaaaaaagaaatgtttataatttgtcaaaaatggctttttttagaatatcaaatagagttataacaataataaatttagggagtttttcttattaatatgatgaaaaaaggaatttttgtaattactatagtgatatctatagcattgataattggaggttttttatagttataattctaggataaataatctttcaaaagctgataaaggaaaagaagttgtaaaaaatagcagtgaaaaaaatcagatagaccttacctataaaaagtattataaaaatttaccaaaatcagttcaaaataaaatagatgatatttcatccaaaaataaagaagttactttaacttgtatttggcaatctgattcagttatttctgaacaatttcaacaaaacttacaaaaatattatggaaataagttttggaacatcaaaaatatcacctacaatggcgaaacaagtgaacaattattggctgaaaaagttcaaaatcaagtattggcgactaaccctgatgttgttttatatgaagctccactttttaatgataaccaatatagactactgggctagttacccagacaaaaattctgatgaaatgaaggggctgttttctgatgatggagtatatagaacattaaatgcttcggggaataaggtttggctagattatattactaaatattttacagcaaactaattaagttataaataacaattattaaatattggagaagaaatgcaggaaacacaggaacagacgattgatttaagagggatttttaaaattattcgcaaaaggttaggtttaatattatttagtgctttaatagtcacaatattagggagcatctacacattttttatagcctccccagtttacacagcctcaactcaacttgtcgttaaactaccaaattcggataattcagcagcctacgctggagaagtgaccgggaatattcaaatggcgaacacaattaaccaagttattgttagtccagtcattttagataaagttcaaagtaatttaaatctatctgatgactctttccaaaaacaagttacagcagcaaatcaaacaaattcacaagttattatgcttactgttaaatattctaatccttacattgcaaaaaagattgcagacgagactgctaaaatttttagttcagatgcagcaaaactattgaatgttactaacgttaatattctatccaaagcaaaagctcaaacaacaccaattagtcctaaacctaaattgtatttagcgatatctgttatagccggactagttttaggtttagccattgctttattgaaggaattgtttgataacaaaattaataaagaagaagatattgaagctctggggctcacggttcttggtgtaacaagctatgatcaaatgagtgattttaataagaatacaaataaaaatggcacgcaatcgggaactaagtcaagtccgcctagcgaccatgaagtaaatagatcatcaaaaaggaataaaagataggagttcaggatggctaaaaataaaagaagcatagacaataatcattatattattaccagtgtcaatcctcaatcacctatttccgaacaatatcgtacgattcgtacgaccattgattttaaaatggcggatcaaggaattaaaagttttctagtaacatcttcagaaacagatgaaggtaaaacaaccgtaagtgctaatatagctgttgcttttgcacaacaaggtaaaaaagtacttttaattgatggcgatcttcgtaaaccgactgttaacattacttttaaagtacaaaatagagtaggattaaccaatattttaatgcatcaatcttcgattgaagatgccatacaagggacaagactttctgaaaatcttacaataattacctctggtccaattccacctaatccatcggaattattagcatctagtgcaatgaagaatttgattgactctgtgtccgatttctttgatgttgttttgattgatattccacctctctctgcagttactgatgctcaaattttgagtagttatgtaggaggagtggttcttgttgtacgtgcctatgaaacaaaaaaagagagtttagcaaaaacaaaaaaaaagctggaacaagttaatgcaaatatattaggagttgttttgcatggggtagactcttctgactcaccgtcgtattactactacggagtagagtaattggaataaattttaatcaaataaaagacagaaatttgtagaagaggagagcaaatgattgatattcattgccatattttaccgggtatagatgatggagctaaaacttctggagatactttgacaatgctgaaatcagcaattgatgaagggataacaaccatcaccgctactcctcatcataatcctcaatttaataatgaatcaccacttattttaaaaaaagttaaggaagttcaaaatatcattgacgagcatcaattaccaattgaagttttgcctggacaagaggttagaatatatggtgatttattaaaagaattttctgaaggaaagttactgaaagcagcgggcacttcaagttatatattgattgaatttccatcaaatcatgtgccagcttatgctaaagaacttttttataatattaaattggagggccttcaacctattttggtccaccctgagcgtaatagtggaatcattgagaaccctgatatattatttgattttattgaacaaggagtactaagtcagataacagcttcaagtgtcactggtcattttggtaaaaaaatacaaaagctatcatttaaaatgatagaaaaccatcttacgcattttgttgcatcagatgcgcataatgtgacgtcacgtgcatttaagatgaaggaagcatttgaaattattgaagatagttatggttctggtgtatcacgaatgttacaaaataatgcagactcggtgattttgaacgaaagtttttatcaagaagaaccaataaaaattaaaacaaagaaatttttgggattattttaaaaggattaaatggagtaaataatggaagtttttgaggcatcatctgaactggaagagcctaagttagtagaattaaaaaaattttctcgcagagagataattataaaaagagggattgatattttagggggattagcgggttcaggtttatttcttatcgcggctgcattgctttatgtcccttacaaaatgagctcaaaaaaagatcaagggccaatgttctataaacaaaaacggtatggaaaaaatggtaaaattttttatattttgaaatttagaacaatgataattaatgctgagcagtatttagagctacatccagaagttaaagccgcctatcatgccaatggcaataaactagaaagtgatccccgtgtaacgaagattgggtcatttattagacaacactcaattgatgaattaccacaatttatcaatgtccttaaaggagatatgtcattagttggtccaagaccaattttgctttttgaagcgaaagaatatggggagcgcctctcttacttactgatatgcaaacctggaattactggttattggacaacacatggtcgaagtaaagttctttttcctcaacgagcagatttagagctctattatctccagtaccatagtacaaaaaatgatataaaacttattatgcttacaataaaacaaattctacatggatcggatgcttattaaagtaacattatgaaaaaaaaaacaactaaaatttgcatgatttcttcttctgggggtcatttaaaagagcttaatgaattgatagagatttcagagcagtatgaaacgtttcaaattactgaaaaagataaattttctaatatcaagattggaactaggcaatactatgtgaataaaattgatagagatgaaaaaaattttttatttcatttttttattctttttttgaaaatatttcaaatatttgctgtagagaagcctaaagttatagtaaccactggtgccttagtagcttatccagcatgtctaataggaaaattaatgagagctaaagttatttttatagagtcttatgctcgaacagaaacattatcattaacaggaaaattagtttataggttatctgatttatttattgttcaatggccagatctttcaaaaaaatattctaaagctaaatactatggggaattattctgatgatattaataatattagggactcaaaaatttcaattcaaccgacttataaaaaaagttgataaattaatagaagatgatcaaatcaaagattctgtaatagctcaaatcggatattctaattacaaacctataaattataaattttcagatttttttgatcaatcggaatttgattcattaataaataaatcagatataataataactcatggaggagtaggtgggatagtttcttccttaaaaaagaataaaaaaatcatagtagttccgcgtttaaagaaatacagagaacatattgatgatcatcaattagagatagcaagggcgtttcaaagaaaaaatctagttattttaaacgagaatctaaatgaactatgtaatgatatatctaaaattgaatcattcgagccaatacactatgtcaaagataataaaaaaattatatgtgaaataaaaaaatttatatcgaaagttaaatgatatttttatacaaaattatcttatgatgagaaaggactttttaaaagataaaaaatgataaaattgagcattataattccaatttataacgtggaaaaatatttaagtaaatgtttaaattctattttagaacaaacttataaagaaatagaaataatattagtaaatgatggtagtactgataactcaaaagatatagctgtaagctattgtgaaagatttcctaatgtttttaaatattttgagaaagataacggaggcctctcttcagccagaaattttggacttgaaaaaatttctggtgattttgtaggcttcttagactcagatgactatatagataacgatttatatgaaattatgattaattcattggatagttcaataaaaattgtggaatgtgattttatatgggaatacgaaaatggaaaaagtgtccttgataaaacatctgaatataattctatcaaagacttaatggttaacggtagagttgttgcttggaataaaatatataatgttgaatggttagaaaaaataaacataaagtttaaagaaggtctattgtatgaagatttaaattttttcttcaaaattgttcctcacttgactagtatttcagaagtatcaacagttaaaaatagttttgttcactatgtccagcataaaggtacaataacttcagataattctcttaatatcttggatatcataaaatcttacgaagatgtctttcattattataacgaaaaacagattaatgatttatattttgatgagctagaatataaattttctaggaacttaatgggggcatttttaaaaagagcaattaagattaaagataaaagacaacgtaaaataattttagatgaattttggaataatgttttatcttactatccgaattggaaaaaaaataaatatataaaaaaactatcaaaacagaatatacttttattttttattaataaatatacatataaattattttatttattataaaaaaaatttaatattagagtatttgtattagttgcaatgaaaatatcgaaagtagaataaatgatttatgtagaaataaggggaaacttaggtaatcaattatttatctatgccaccgcaaaaaaaattcaaaagttaaccggacaaaaaattcaattaaatacaacaactttaaataaatactttccaaattacaagtttggcctttcagaatttataatggaggatcctgattgttttattgaatcctataaaaaattaccctggttcacaaacgagtatctcttacctattaaaatttttaaaaaaatattgaataaaacacccaaaattaataaaatcctttcagatttttttttcaaagcttttgaaaaaaaaggatattttatttggcgaggagagacttttaaaaagttttctttaggaaatcataaaaattactatttatcaggtttttggcaatcggaagaatatttttatgatataagggatgaattattagaaatcatcactcctataaattcaataagagagtgtaactttgaacttctcaatttaataagtaattcagaatcaatttgtgtttcaatacgccgaggagattatgtagataatcctaaaatatcagctatttataacgtatgtgatataaattattttatagaatctgtaaatgaaataaagaaaaatgttgtgaatgttaaagttatctgtttttcagatgatgttgaatgggtcaaaaaaaatataaaattcgactgtgaaacacattatgaaacttatggtaattctttatctgaaaaagttcaacttatgtcttcttgtaaacattttgttttatctaatagttcttttagttggtggacagaatttttatctatacgaggtgggattactatagcccccaaaaattggtatgcagatgaacgtgaagctgatatctatagaaaaaattggatttacttagaagataagacagaggaagagtaatgggatttctatttttaactataatacttattttgtgggggtatagttttaccaatataaaaataagcccttttagtattttattcatgagtttagggatcttttactctcaatttacttcaataaatattgacttaataataaaagtactttttttgataacttccataatttatcttattaaagataaatattcaaaaaaatacgttttttctttattattaattgcagtattaattttaattgagtcaactagtccctctaaatttaatcaatattatggttttattgatgctttgacatcatttgcaaccttctcaacaggcatacttctattttccataaaatttagtttacaagaacgcagaagtattttaaaatcaatttcatatttgccaatcttttcagtgttaattggaatccctctaacttttggtggttttatatctatgacagctagaggaggaattgccctttcaggagcagctttagaaacaaatttatcttttttttcagttctaagccttgtttcattagatattttatatcaggacactcgttctaataaatatcaaattttaaaaattattaactttatattgctatgttgtactttaacacgaggcggtattatttctggaattatcattattttaccaagtttactatttcttttaaaaaaaggatttaaaggagtaagacaatttatttttttgattattactatttttgggagtatttatccgcttattttattgtggaaaagtattagtgagaggactttcagtgcagatggtattaatacttcaggtcgatatacggcctgggactatattgtgaatttgacaacaaacaaatctcagggaatgggattgggaagtttaaagacattaactgaggatattaatttacgtgcctttactgctgctcataatacatatattcaattttattatgaaactggttatttgggagtaacactattatctattttatttattttaatattaataataatcctaaaattgactaattatagaaaaaaaatcatttacttaacattcatttcatttttagtatatagttatacagataattgtattgttaataatagatactggtatttgtttatgtttattataggatgttttaaatattttgacagaaaggaagaaaatgcgctactttaaaatattatttgagattattcaactattggtagctagtattttatgtagattatataaaaatccaaatgatatctggctaataaatgaaaaacctgatgaagctagagataatggttatgctttttatcaatatttaagaaagaatttccccgatattaaagtttattatgtaatcagtaaagagtctactgatatttataagtttgataatgaaactaacattgtattttataagagttttttacattttattttatatatcaaatctaaagttttaattagttctcaaacattgccctatccatctagcagaaaattatgtgaagcgctaatgtaccttaatttgaataaaccaaagaggatttggttacaacatggagttactaaagataaactcccatatgagaatatggcaagggaaatttttaagtatgatttaataacctgtgtttcattaaaagaggctaattttataatgaaagaatatggatataatgaagatcaggtgaaggctcttggatttgcaagatatgataatttgccaattggaaataataacacatttgatatacttataatgcctactttccgtaagggttacgagattaaaaattttagtctcccaacagatagtgaaactaaacattttgaggaaagtgtattctttaaaacatatgttgatttattgaattctgaagagctagacgagtatttagaaaagtctggtaaaaaagcaattttttatttacactatgcttttcaaccatatgcaaaatctttttctaaacgactaatgtcttcaaatgttatcattgctgaaagaacagaatatgatgttcaaaaactattaattaattgtgaattgctaattacagattattcaagcgttttttttgatttttcatatatgaaaaaacctgaaatatttttccattttgatgagaaagaatatagaagtaatcattatagggagggatattttgattataaaacagatggatttggtccagtagttaattctaaagaagaattactaactgaaatcaaagagtttattgataacccatctctgttaatggaatttaataagcgagctaataatttcttcaaatatactgataacaataattgccaacgtattttaaaagaaatttggagaattaatgaaactaattaagaattatttaatgacaagctcttatcaattgttaattattatcttaccaataataacaacaccatatatatcgagagtacttagtccagaagggattggactttattcatatacttatactattacacagtatttcgtattatttgctactcttggtactgttacgtatggtagcagagagatagcatattatcagtcaaataaacaaaagagaagtgaaattttttggggaattaccttccttagctgggctactggtgctatatcacttttaatattttatatatttattttttttaatggcaaatatagtgttttatttttttggcaaagctttttgatttttggagttatttttgatattaattggtatttcacaggaatggaaaagttcaaggttattatttcacgtaacttttgtataaaaattattagtttattgtgtatttttgtctttgtaaaatctgagaaagatttaagtttatatatagttatactaggattgagcaatataataggtaatatattagtttggccatatttgagaaaagaggtttataaacctaatttttctaagttatcattcaaaaaacatttgggaagtacatggatattttttttgccacaaacttctgttactttaaactcattaataaaccaaaatatgattgcatattttgactcaataacaagcttaggatactttacacaaacaaataagtttactgtgattgcgatttcaatagttatttcaattgggactgttatgttgcctagaatgtccaatttagttgcgcgcaaagagtattcaaagtttacagactatgttactaagagtattaatataagctcaggaatttctatagcaataatgtttggtttaatggctatagcacctaagtttacaactttttttttaggagctcaatataaatttgttattcatttgctagttttatcatcaccgatagtggttttagtaacctggagtaatgttcttggtcaacaatatttaatacctttaaataggatgaaaatatttacaaaatctctaatttgtggaaacttagtaaatgtttctctaaacttgattttgttacccaaaatgggagtagaaatttcaataataaatcagttaattaatgaaattattattgtaggtattcaatttatatcagttagaaaagagttaaaaataaatataatattaggagatctaataaaatatttttttgcgggtataattatgtttattgccgttttatatctgaatttacaattaccgatgactatcttcacactacttatagagattggtattggagttcttatatattcaatgctagttatttctcttaaaactggattatataaagaattgaaaaagattattaaaattcgttagcttaaaatctatcacctttcatttgagtagtaagaaatacaaagctttattataaaatttatcatttttaagactatcataaaagaagaaggatgacatggaacgaaaaaagaagaaaaaaaaaatatatataattattctaatattattaatgtttatcactattgtttgttttgggggatatgctacacgagagttaattactcccactgaaaaaacaataccaaatgtctcggatcaacctaaaaaaacttcggcctctaacggttatgtagagcaaaaaggggaagaagctgctgtgggtagtatagcacttgtagacgatgctggtgtaccagaatgggttaaagttccctcaaaggtaaatctagataaatttactgatttatctacgaataatatcactatttatcgaattaataatccggaagtcttaaaaacagttaccaatcgtacagatcaacggatgaaaatgtcagaagttatagctaagtatcctaatgctttgattatgaatgcttccgcttttgatatgcagacaggacaagtagctggatttcaaattaataatggaaagttgattcaagactggagtccaggtacaacgactcaatatgcttttgttattaacaaagatggttcgtgcaaaatttatgattcaagtacacctgctttaactattattaaaaatggagggcaacaagcctatgattttggtactgcgattatccgtgatggtaaaattcaaccaagtgatggctcagtagattggaagattcatatttttattgcgaatgataaagataataatctctatgctattttgagtgatacaaatgcaggttatgataatataataaaatcagtatcaaatttgaagctccaaaatatgttattacttgatagtggtggctcaagtcaactatctgtcaatggtaaaacgattgttgctagtcaagatgatcgagccgtaccggattatattgtgatgaaataaaaataaaagaacctcatggttcttttattttagagatttttcaaaaagggttttgactgagtctaattctgtttgagaaacgaccttagctccattttcatctgttgtatgtagattgagcttgctggtattctttagagccttactgtagctcataacgatcgttttagcatcattgaaacttatattggttttaacagtgtttgaaaaagcatagagaatttctcgatagtaattgaaactttcaagttttttcatatgagcaattttttgaatatttccgtagagttccattgagacattttgaatccgagtgattgaagcatccaaatcagtatcgtcaatttgtgtcatataggcttggacttgatcagcagtttgtaaattaacagttccttgtttaaactcataaccttcagccttgaatgcctttggattttgcatggtgattccaccagtggcctgtacaagtgatcccattttattaacatcgatctgaattactttgttaatggacgcattcaataggtctttaaccatctggaaaattccatcatctccattcgtattgtaaacttcagtgattgttttttgattaggcattgtcgcaaaaactgggaagttcatgaaagtagtttgatttgtctttacattcgttgaagctaaaacagtagcataagctgaatttttagaattatttttaccagttgcaatgataagtgtggtgaatgttttagctttttttaagtcaatacttgttgttttagggaaattttcatatgatgttgaaaaggttgattcaacatttctataagctacgtaggctatagaagcaacagaaataattactaatacaaaaataattgaaataacttttagtactgtgtattttttcttacgataatgacgcctctttttttgattcat

SEQ ID NO: 4 - DNA, 12651 bp, Lactococcus lactis
  Eps gene cluster of 33225
atgaatgatttattttaccatcgtctaaaggaactagttgaatcaagtggtaaatctgcaaatcaaatagaaagggaattgggttaccctagaaattctttgaataattataagttgggaggagaaccctctgggacaagattaataggacaatcagagtattttaatgtgtctccaaaatatctgatgggtataagtgatgagcctaatgacagttctgcaattaatctttttaaaactctaactcaagaagagaaaaaagaaatgtttataatttgtcaaaaatggctttttttagaatatcaaatagagttataacaataataaatttagggagtttttcttattaatatgatgaaaaaaggaatttttgtaattactatagtgatatctatagcattgataattggaggttttttatagttataattctaggataaataatctttcaaaagctgataaaggaaaagaagttgtaaaaaatagcagtgaaaaaaatcagatagaccttacctataaaaagtattataaaaatttaccaaaatcagttcaaaataaaatagatgatatttcatccaaaaataaagaagttactttaacttgtatttggcaatctgattcagttatttctgaacaatttcaacaaaacttacaaaaatattatggaaataagttttggaacatcaaaaatatcacctacaatggcgaaacaagtgaacaattattggctgaaaaagttcaaaatcaagtattggcgactaaccctgatgttgttttatatgaagctccactttttaatgataaccaatatagactactgggctagttacccagacaaaaattctgatgaaatgaaggggctgttttctgatgatggagtatatagaacattaaatgcttcggggaataaggtttggctagattatattactaaatattttacagcaaactaattaagttataaataacaattattaaatattggagaagaaatgcaggaaacacaggaacagacgattgatttaagagggatttttaaaattattcgcaaaaggttaggtttaatattatttagtgctttaatagtcacaatattagggagcatctacacattttttatagcctccccagtttacacagcctcaactcaacttgtcgttaaactaccaaattcggataattcagcagcctacgctggagaagtgaccgggaatattcaaatggcgaacacaattaaccaagttattgttagtccagtcattttagataaagttcaaagtaatttaaatctatctgatgactctttccaaaaacaagttacagcagcaaatcaaacaaattcacaagttattatgcttactgttaaatattctaatccttacattgcaaaaaagattgcagacgagactgctaaaatttttagttcagatgcagcaaaactattgaatgttactaacgttaatattctatccaaagcaaaagctcaaacaacaccaattagtcctaaacctaaattgtatttagcgatatctgttatagccggactagttttaggtttagccattgctttattgaaggaattgtttgataacaaaattaataaagaagaagatattgaagctctggggctcacggttcttggtgtaacaagctatgatcaaatgagtgattttaataagaatacaaataaaaatggcacgcaatcgggaactaagtcaagtccgcctagcgaccatgaagtaaatagatcatcaaaaaggaataaaagataggagttcaggatggctaaaaataaaagaagcatagacaataatcattatattattaccagtgtcaatcctcaatcacctatttccgaacaatatcgtacgattcgtacgaccattgattttaaaatggcggatcaaggaattaaaagttttctagtaacatcttcagaaacagatgaaggtaaaacaaccgtaagtgctaatatagctgttgcttttgcacaacaaggtaaaaaagtacttttaattgatggcgatcttcgtaaaccgactgttaacattacttttaaagtacaaaatagagtaggattaaccaatattttaatgcatcaatcttcgattgaagatgccatacaagggacaagactttctgaaaatcttacaataattacctctggtccaattccacctaatccatcggaattattagcatctagtgcaatgaagaatttgattgactctgtgtccgatttctttgatgttgttttgattgatattccacctctctctgcagttactgatgctcaaattttgagtagttatgtaggaggagtggttcttgttgtacgtgcctatgaaacaaaaaaagagagtttagcaaaaacaaaaaaaaagctggaacaagttaatgcaaatatattaggagttgttttgcatggggtagactcttctgactcaccgtcgtattactactacggagtagagtaattggaataaattttaatcaaataaaagacagaaatttgtagaagaggagagcaaatgattgatattcattgccatattttaccgggtatagatgatggagctaaaacttctggagatactttgacaatgctgaaatcagcaattgatgaagggataacaaccatcaccgctactcctcatcataatcctcaatttaataatgaatcaccacttattttaaaaaaagttaaggaagttcaaaatatcattgacgagcatcaattaccaattgaagttttgcctggacaagaggttagaatatatggtgatttattaaaagaattttctgaaggaaagttactgaaagcagcgggcacttcaagttatatattgattgaatttccatcaaatcatgtgccagcttatgctaaagaacttttttataatattaaattggagggccttcaacctattttggtccaccctgagcgtaatagtggaatcattgagaaccctgatatattatttgattttattgaacaaggagtactaagtcagataacagcttcaagtgtcactggtcattttggtaaaaaaatacaaaagctatcatttaaaatgatagaaaaccatcttacgcattttgttgcatcagatgcgcataatgtgacgtcacgtgcatttaagatgaaggaagcatttgaaattattgaagatagttatggttctggtgtatcacgaatgttacaaaataatgcagactcggtgattttgaacgaaagtttttatcaagaagaaccaataaaaattaaaacaaagaaatttttgggattattttaaaaggattaaatggagtaaataatggaagtttttgaggcatcatctgaactggaagagcctaagttagtagaattaaaaaaattttctcgcagagagataattataaaaagagggattgatattttagggggattagcgggttcaggtttatttcttatcgcggctgcattgctttatgtcccttacaaaatgagctcaaaaaaagatcaagggccaatgttctataaacaaaaacggtatggaaaaaatggtaaaattttttatattttgaaatttagaacaatgataattaatgctgagcagtatttagagctacatccagaagttaaagccgcctatcatgccaatggcaataaactagaaagtgatccccgtgtaacgaagattgggtcatttattagacaacactcaattgatgaattaccacaatttatcaatgtccttaaaggagatatgtcattagttggtccaagaccaattttgctttttgaagcgaaagaatatggggagcgcctctcttacttactgatatgcaaacctggaattactggttattggacaacacatggtcgaagtaaagttctttttcctcaacgagcagatttagagctctattatctccagtaccatagtacaaaaaatgatataaaacttattatgcttacaataaaacaaattctacatggatcggatgcttattaaagtaacattatgaaaaaaaaaacaactaaaatttgcatgatttcttcttctgggggtcatttaaaagagcttaatgaattgatagagatttcagagcagtatgaaacgtttcaaattactgaaaaagataaattttctaatatcaagattggaactaggcaatactatgtgaataaaattgatagagatgaaaaaaattttttatttcatttttttattctttttttgaaaatatttcaaatatttgctgtagagaagcctaaagttatagtaaccactggtgccttagtagcttatccagcatgtctaataggaaaattaatgagagctaaagttatttttatagagtcttatgctcgaacagaaacattatcattaacaggaaaattagtttataggttatctgatttatttattgttcaatggccagatctttcaaaaaaatattctaaagctaaatactatggggaattattctgatgatattaataatattagggactcaaaaatttcaattcaaccgacttataaaaaaagttgataaattaatagaagatgatcaaatcaaagattctgtaatagctcaaatcggatattctaattacaaacctataaattataaattttcagatttttttgatcaatcggaatttgattcattaataaataaatcagatataataataactcatggaggagtaggtgggatagtttcttccttaaaaaagaataaaaaaatcatagtagttccgcgtttaaagaaatacagagaacatattgatgatcatcaattagagatagcaagggcgtttcaaagaaaaaatctagttattttaaacgagaatctaaatgaactatgtaatgatatatctaaaattgaatcattcgagccaatacactatgtcaaagataataaaaaaattatatgtgaaataaaaaaatttatatcgaaagttaaatgatatttttatacaaaattatcttatgatgagaaaggactttttaaaagataaaaaatgataaaattgagcattataattccaatttataacgtggaaaaatatttaagtaaatgtttaaattctattttagaacaaacttataaagaaatagaaataatattagtaaatgatggtagtactgataactcaaaagatatagctgtaagctattgtgaaagatttcctaatgtttttaaatattttgagaaagataacggaggcctctcttcagccagaaattttggacttgaaaaaatttctggtgattttgtaggcttcttagactcagatgactatatagataacgatttatatgaaattatgattaattcattggatagttcaataaaaattgtggaatgtgattttatatgggaatacgaaaatggaaaaagtgtccttgataaaacatctgaatataattctatcaaagacttaatggttaacggtagagttgttgcttggaataaaatatataatgttgaatggttagaaaaaataaacataaagtttaaagaaggtctattgtatgaagatttaaattttttcttcaaaattgttcctcacttgactagtatttcagaagtatcaacagttaaaaatagttttgttcactatgtccagcataaaggtacaataacttcagataattctcttaatatcttggatatcataaaatcttacgaagatgtctttcattattataacgaaaaacagattaatgatttatattttgatgagctagaatataaattttctaggaacttaatgggggcatttttaaaaagagcaattaagattaaagataaaagacaacgtaaaataattttagatgaattttggaataatgttttatcttactatccgaattggaaaaaaaataaatatataaaaaaactatcaaaacagaatatacttttattttttattaataaatatacatataaattattttatttattataaaaaaaatttaatattagagtatttgtattagttgcaatgaaaatatcgaaagtagaataaatgatttatgtagaaataaggggaaacttaggtaatcaattatttatctatgccaccgcaaaaaaaattcaaaagttaaccggacaaaaaattcaattaaatacaacaactttaaataaatactttccaaattacaagtttggcctttcagaatttataatggaggatcctgattgttttattgaatcctataaaaaattaccctggttcacaaacgagtatctcttacctattaaaatttttaaaaaaatattgaataaaacacccaaaattaataaaatcctttcagatttttttttcaaagcttttgaaaaaaaaggatattttatttggcgaggagagacttttaaaaagttttctttaggaaatcataaaaattactatttatcaggtttttggcaatcggaagaatatttttatgatataagggatgaattattagaaatcatcactcctataaattcaataagagagtgtaactttgaacttctcaatttaataaggaattcagaatcaatttgtgtttcaatacgccgaggagattatgtagataatcctaaaatatcagctatttataacgtatgtgatataaattattttatagaatctgtaaatgaaataaagaaaaatgttgtgaatgttaaagttatctgtttttcagatgatgttgaatgggtcaaaaaaaatataaaattcgactgtgaaacacattatgaaacttatggtaattctttatctgaaaaagttcaacttatgtcttcttgtaaacattttgttttatctaatagttcttttagttggtggacagaatttttatctatacgaggtgggattactatagcccccaaaaattggtatgcagatgaacgtgaagctgatatctatagaaaaaattggatttacttagaagataagacagaggaagagtaatgggatttctatttttaactataatacttattttgtgggggtatagttttaccaatataaaaataagcccttttagtattttattcatgagtttagggatcttttactctcaatttacttcaataaatattgacttaataataaaagtactttttttgataacttccataatttatcttattaaagataaatattcaaaaaaatacgttttttctttattattaattgcagtattaattttaattgagtcaactagtccctctaaatttaatcaatattatggttttattgatgctttgacatcatttgcaaccttctcaacaggcatacttctattttccataaaatttagtttacaagaacgcagaagtattttaaaatcaatttcatatttgccaatcttttcagtgttaattggaatccctctaacttttggtggttttatatctatgacagctagaggaggaattgccctttcaggagcagctttagaaacaaatttatcttttttttcagttctaagccttgtttcattagatattttatatcaggacactcgttctaataaatatcaaattttaaaaattattaactttatattgctatgttgtactttaacacgaggcggtattatttctggaattatcattattttaccaagtttactatttcttttaaaaaaaggatttaaaggagtaagacaatttatttttttgattattactatttttgggagtatttatccgcttattttattgtggaaaagtattagtgagaggactttcagtgcagatggtattaatacttcaggtcgatatacggcctgggactatattgtgaatttgacaacaaacaaatctcagggaatgggattgggaagtttaaagacattaactgaggatattaatttacgtgcctttactgctgctcataatacatatattcaattttattatgaaactggttatttgggagtaacactattatctattttatttattttaatattaataataatcctaaaattgactaattatagaaaaaaaatcatttacttaacattcatttcatttttagtatatagttatacagataattgtattgttaataatagatactggtatttgtttatgtttattataggatgttttaaatattttgacagaaaggaagaaaatgcgctactttaaaatattatttgagattattcaactattggtagctagtattttatgtagattatataaaaatccaaatgatatctggctaataaatgaaaaacctgatgaagctagagataatggttatgctttttatcaatatttaagaaagaatttccccgatattaaagtttattatgtaatcagtaaagagtctactgatatttataagtttgataatgaaactaacattgtattttataagagttttttacattttattttatatatcaaatctaaagttttaattagttctcaaacattgccctatccatctagcagaaaattatgtgaagcgctaatgtaccttaatttgaataaaccaaagaggatttggttacaacatggagttactaaagataaactcccatatgagaatatggcaagggaaatttttaagtatgatttaataacctgtgtttcattaaaagaggctaattttataatgaaagaatatggatataatgaagatcaggtgaaggctcttggatttgcaagatatgataatttgccaattggaaataataacacatttgatatacttataatgcctactttccgtaagggttacgagattaaaaattttagtctcccaacagatagtgaaactaaacattttgaggaaagtgtattctttaaaacatatgttgatttattgaattctgaagagctagacgagtatttagaaaagtctggtaaaaaagcaattttttatttacactatgcttttcaaccatatgcaaaatctttttctaaacgactaatgtcttcaaatgttatcattgctgaaagaacagaatatgatgttcaaaaactattaattaattgtgaattgctaattacagattattcaagcgttttttttgatttttcatatatgaaaaaacctgaaatatttttccattttgatgagaaagaatatagaagtaatcattatagggagggatattttgattataaaacagatggatttggtccagtagttaattctaaagaagaattactaactgaaatcaaagagtttattgataacccatctctgttaatggaatttaataagcgagctaataatttcttcaaatatactgataacaataattgccaacgtattttaaaagaaatttggagaattaatgaaactaattaagaattatttaatgacaagctcttatcaattgttaattattatcttaccaataataacaacaccatatatatcgagagtacttagtccagaagggattggactttattcatatacttatactattacacagtatttcgtattatttgctactcttggtactgttacgtatggtagcagagagatagcatattatcagtcaaataaacaaaagagaagtgaaattttttggggaattaccttccttagctgggctactggtgctatatcacttttaatattttatatatttattttttttaatggcaaatatagtgttttatttttttggcaaagctttttgatttttggagttatttttgatattaattggtatttcacaggaatggaaaagttcaaggttattatttcacgtaacttttgtataaaaattattagtttattgtgtatttttgtctttgtaaaatctgagaaagatttaagtttatatatagttatactaggattgagcaatataataggtaatatattagtttggccatatttgagaaaagaggtttataaacctaatttttctaagttatcattcaaaaaacatttgggaagtacatggatattttttttgccacaaacttctgttactttaaactcattaataaaccaaaatatgattgcatattttgactcaataacaagcttaggatactttacacaaacaaataagtttactgtgattgcgatttcaatagttatttcaattgggactgttatgttgcctagaatgtccaatttagttgcgcgcaaagagtattcaaagtttacagactatgttactaagagtattaatataagctcaggaatttctatagcaataatgtttggtttaatggctatagcacctaagtttacaactttttttttaggagctcaatataaatttgttattcatttgctagttttatcatcaccgatagtggttttagtaacctggagtaatgttcttggtcaacaatatttaatacctttaaataggatgaaaatatttacaaaatctctaatttgtggaaacttagtaaatgtttctctaaacttgattttgttacccaaaatgggagtagaaatttcaataataaatcagttaattaatgaaattattattgtaggtattcaatttatatcagttagaaaagagttaaaaataaatataatattaggagatctaataaaatatttttttgcgggtataattatgtttattgccgttttatatctgaatttacaattaccgatgactatcttcacactacttatagagattggtattggagttcttatatattcaatgctagttatttctcttaaaactggattatataaagaattgaaaaagattattaaaattcgttagcttaaaatctatcacctttcatttgagtagtaagaaatacaaagctttattataaaatttatcatttttaagactatcataaaagaagaaggatgacatggaacgaaaaaagaagaaaaaaaaaatatatataattattctaatattattaatgtttatcactattgtttgttttgggggatatgctacacgagagttaattactcccactgaaaaaacaataccaaatgtctcggatcaacctaaaaaaacttcggcctctaacggttatgtagagcaaaaaggggaagaagctgctgtgggtagtatagcacttgtagacgatgctggtgtaccagaatgggttaaagttccctcaaaggtaaatctagataaatttactgatttatctacgaataatatcactatttatcgaattaataatccggaagtcttaaaaacagttaccaatcgtacagatcaacggatgaaaatgtcagaagttatagctaagtatcctaatgctttgattatgaatgcttccgcttttgatatgcagacaggacaagtagctggatttcaaattaataatggaaagttgattcaagactggagtccaggtacaacgactcaatatgcttttgttattaacaaagatggttcgtgcaaaatttatgattcaagtacacctgctttaactattattaaaaatggagggcaacaagcctatgattttggtactgcgattatccgtgatggtaaaattcaaccaagtgatggctcagtagattggaagattcatatttttattgcgaatgataaagataataatctctatgctattttgagtgatacaaatgcaggttatgataatataataaaatcagtatcaaatttgaagctccaaaatatgttattacttgatagtggtggctcaagtcaactatctgtcaatggtaaaacgattgttgctagtcaagatgatcgagccgtaccggattatattgtgatgaaataaaaataaaagaacctcatggttcttttattttagagatttttcaaaaagggttttgactgagtctaattctgtttgagaaacgaccttagctccattttcatctgttgtatgtagattgagcttgctggtattctttagagccttactgtagctcataacgatcgttttagcatcattgaaacttatattggttttaacagtgtttgaaaaagcatagagaatttctcgatagtaattgaaactttcaagttttttcatatgagcaattttttgaatatttccgtagagttccattgagacattttgaatccgagtgattgaagcatccaaatcagtatcgtcaatttgtgtcatataggcttggacttgatcagcagtttgtaaattaacagttccttgtttaaactcataaccttcagccttgaatgcctttggattttgcatggtgattccaccagtggcctgtacaagtgatcccattttattaacatcgatctgaattactttgttaatggacgcattcaataggtctttaaccatctggaaaattccatcatctccattcgtattgtaaacttcagtgattgttttttgattaggcattgtcgcaaaaactgggaagttcatgaaagtagtttgatttgtctttacattcgttgaagctaaaacagtagcataagctgaatttttagaattatttttaccagttgcaatgataagtgtggtgaatgttttagctttttttaagtcaatacttgttgttttagggaaattttcatatgatgttgaaaaggttgattcaacatttctataagctacgtaggctatagaagcaacagaaataattactaatacaaaaataattgaaataacttttagtactgtgtattttttcttacgataatgacgcctctttttttgattcat

SEQ ID NO: 5 - DNA, 12651 bp, Lactococcus lactis
  Eps gene cluster of 33133
atgaatgatttattttaccatcgtctaaaggaactagttgaatcaagtggtaaatctgcaaatcaaatagaaagggaattgggttaccctagaaattctttgaataattataagttgggaggagaaccctctgggacaagattaataggactatcagagtattttaatgtgtctccaaaatatctgatgggtataagtgatgagcctaatgacagttctgcaattaatctttttaaaactctaactcaagaagagaaaaaagaaatgtttataatttgtcaaaaatggctttttttagaatatcaaatagagttataacaataataaatttagggagtttttcttattaatatgatgaaaaaaggaatttttgtaattactatagtgatatctatagcattgataattggaggttttttatagttataattctaggataaataatctttcaaaagctgataaaggaaaagaagttgtaaaaaatagcagtgaaaaaaatcagatagaccttacctataaaaagtattataaaaatttaccaaaatcagttcaaaataaaatagatgatatttcatccaaaaataaagaagttactttaacttgtatttggcaatctgattcagttatttctgaacaatttcaacaaaacttacaaaaatattatggaaataagttttggaacatcaaaaatatcacctacaatggcgaaacaagtgaacaattattggctgaaaaagttcaaaatcaagtattggcgactaaccctgatgttgttttatatgaagctccactttttaatgataaccaatatagactactgggctagttacccagacaaaaattctgatgaaatgaaggggctgttttctgatgatggagtatatagaacattaaatgcttcggggaataaggtttggctagattatattactaaatattttacagcaaactaattaagttataaataacaattattaaatattggagaagaaatgcaggaaacacaggaacagacgattgatttaagagggatttttaaaattattcgcaaaaggttaggtttaatattatttagtgctttaatagtcacaatattagggagcatctacacattttttatagcctccccagtttacacagcctcaactcaacttgtcgttaaactaccaaattcggataattcagcagcctacgctggagaagtgaccgggaatattcaaatggcgaacacaattaaccaagttattgttagtccagtcattttaggtaaagttcaaagtaatttaaatctatctgatgactctttccaaaaacaagttacagcagcaaatcaaacaaattcacaagttattacgcttactgttaaatattctaatccttacattgcaaaaaagattgcagacgagactgctaaaatttttagttcagatgcagcaaaactattgaatgttactaacgttaatattctatccaaagcaaaagctcaaacaacaccaattagtcctaaacctaaattgtatttagcgatatctgttatagccggactagttttaggtttagccattgctttattgaaggaattgtttgataacaaaattaataaagaagaagatattgaagctctggggctcacggttcttggtgtaacaagctatgatcaaatgagtgattttaataagaatacaaataaaaatggcacgcaatcgggaactaagtcaagtccgcctagcgaccatgaagtaaatagatcatcaaaaaggaataaaagataggagttcaggatggctaaaaataaaagaagcatagacaataatcattatattattaccagtgtcaatcctcaatcacctatttccgaacaatatcgtacgattcgtacgaccattgattttaaaatggcggatcaaggaattaaaagttttctagtaacatcttcagaaacagatgaaggtaaaacagccgtaagtgctaatatagctgttgcttttgcacaacaaggtaaaaaagtacttttaattgatggcgatcttcgtaaaccgactgttaacattacttttaaagtacaaaatagagtaggattaaccaatattttaatgcatcaatcttcgattgaagatgccatacaagggacaagactttctgaaaatcttacaataattacctctggtccaattccacctaatccatcggaattattagcatctagtgcaatgaagaatttgattgactctgtgtccgatttctttgatgttgttttgattgatactccacctctctctgcagttactgatgctcaaattttgagtagttatgtaggaggagtggttcttgttgtacgtgcctatgaaacaaaaaaagagagtttagcaaaaacaaaaaaaaagctggaacaagttaatgcaaatatattaggagttgttttgcatggggtagactcttctgactcaccgtcgtattactactacggagtagagtaattggaataaattttaatcaaataaaagacagaaatttgtagaagaggagagcaaatgattgatattcattgccatattttaccgggtatagatgatggagctaaaacttctggagatactttgacaatgctgaaatcagcaattgatgaagggataacaaccatcaccgctactcctcatcataatcctcaatttaataatgaatcaccacttattttaaaaaaagttaaggaagttcaaaatatcattgacgagcatcaattaccaattgaagttttgcctggacaagaggttagaatatatggtgatttattaaaagaattttctgaaggaaagttactgaaagcagcgggcacttcaagttatatattgattgaatttccatcaaatcatgtgccagcttatgctaaagaacttttttataatattaaattggagggccttcaacctattttggtccaccctgagcgtaatagtggaatcattgagaaccctgatatattatttgattttattgaacaaggagtactaagtcagataacagcttcaagtgtcactggtcattttggtaaaaaaatacaaaagctatcatttaaaatgatagaaaaccatcttacgcattttgttgcatcagatgcgcataatgtgacgtcacgtgcatttaagatgaaggaagcatttgaaattattgaagatagttatggttctggtgtatcacgaatgttacaaaataatgcagactcggtgattttgaacgaaagtttttatcaagaagaaccaataaaaattaaaacaaagaaatttttgggattattttaaaaggattaaatggagtaaataatggaagtttttgaggcatcatctgaactggaagagcctaagttagtagaattaaaaaaattttctcgcagagagataattataaaaagagggattgatattttagggggattagcgggttcaggtttatttcttatcgcggctgcattgctttatgtcccttacaaaatgagctcaaaaaaagatcaagggccaatgttctataaacaaaaacggtatggaaaaaatggtaaaattttttatattttgaaatttagaacaatgataattaatgctgagcagtatttagagctacatccagaagttaaagccgcctatcatgccaatggcaataaactagaaagtgatccccgtgtaacgaagattgggtcatttattagacaacactcaattgatgaattaccacaatttatcaatgtccttaaaggagatatgtcattagttggtccaagaccaattttgctttttgaagcgaaagaatatggggagcgcctctcttacttactgatatgcaaacctggaattactggttattggacaacacatggtcgaagtaaagttctttttcctcaacgagcagatttagagctctattatctccagtaccatagtacaaaaaatgatataaaacttattatgcttacaataaaacaaattctacatggatcggatgcttattaaagtaacattatgaaaaaaaaaacaactaaaatttgcatgatttcttcttctgggggtcatttaaaagagcttaatgaattgatagagatttcagagcagtatgaaacgtttcaaattactgaaaaagataaattttctaatatcaagattggaactaggcaatactatgtgaataaaattgatagagatgaaaaaaattttttatttcatttttttattctttttttgaaaatatttcaaatatttgctgtagagaagcctaaagttatagtaaccactggtgccttagtagcttatccagcatgtctaataggaaaattaatgagagctaaagttatttttatagagtcttatgctcgaacagaaacattatcattaacaggaaaattagtttataggttatctgatttatttattgttcaatggccagatctttcaaaaaaatattctaaagctaaatactatggggaattattctgatgatattaataatattagggactcaaaaatttcaattcaaccgacttataaaaaaagttgataaattaatagaagatgatcaaatcaaagattctgtaatagctcaaatcggatattctaattacaaacctataaattataaattttcagatttttttgatcaatcggaatttgattcattaataaataaatcagatataataataactcatggaggagtaggtgggatagtttcttccttaaaaaagaataaaaaaatcatagtagttccgcgtttaaagaaatacagagaacatattgatgatcatcaattagagatagcaagggcgtttcaaagaaaaaatctagttattttaaacgagaatctaaatgaactatgtaatgatatatctaaaattgaatcattcgagccaatacactatgtcaaagataataaaaaaattatatgtgaaataaaaaaatttatatcgaaagttaaatgatatttttatacaaaattatcttatgatgagaaaggactttttaaaagataaaaaatgataaaattgagcattataattccaatttataacgtggaaaaatatttaagtaaatgtttaaattctattttagaacaaacttataaagaaatagaaataatattagtaaatgatggtagtactgataactcaaaagatatagctgtaagctattgtgaaagatttcctaatgtttttaaatattttgagaaagataacggaggcctctcttcagccagaaattttggacttgaaaaaatttctggtgattttgtaggcttcttagactcagatgactatatagataacgatttatatgaaattatgattaattcattggatagttcaataaaaattgtggaatgtgattttatatgggaatacgaaaatggaaaaagtgtccttgataaaacatctgaatataattctatcaaagacttaatggttaacggtagagttgttgcttggaataaaatatataatgttgaatggttagaaaaaataaacataaagtttaaagaaggtctattgtatgaagatttaaattttttcttcaaaattgttcctcacttgactagtatttcagaagtatcaacagttaaaaatagttttgttcactatgtccagcataaaggtacaataacttcagataattctcttaatatcttggatatcataaaatcttacgaagatgtctttcattattataacgaaaaacagattaatgatttatattttgatgagctagaatataaattttctaggaacttaatgggggcatttttaaaaagagcaattaagattaaagataaaagacaacgtaaaataattttagatgaattttggaataatgttttatcttactatccgaattggaaaaaaaataaatatataaaaaaactatcaaaacagaatatacttttattttttattaataaatatacatataaattattttatttattataaaaaaaatttaatattagagtatttgtattagttgcaatgaaaatatcgaaagtagaataaatgatttatgtagaaataaggggaaacttaggtaatcaattatttatctatgccaccgcaaaaaaaattcaaaagttaaccggacaaaaaattcaattaaatacaacaactttaaataaatactttccaaattacaagtttggcctttcagaatttataatggaggatcctgattgttttattgaatcctataaaaaattaccctggttcacaaacgagtatctcttacctattaaaatttttaaaaaaatattgaataaaacacccaaaattaataaaatcctttcagatttttttttcaaagcttttgaaaaaaaaggatattttatttggcgaggagagacttttaaaaagttttctttaggaaatcataaaaattactatttatcaggtttttggcaatcggaagaatatttttatgatataagggatgaattattagaaatcatcactcctataaattcaataagagagtgtaactttgaacttctcaatttaataaggaattcagaatcaatttgtgtttcaatacgccgaggagattatgtagataatcctaaaatatcagctatttataacgtatgtgatataaattattttatagaatctgtaaatgaaataaagaaaaatgttgtgaatgttaaagttatctgtttttcagatgatgttgaatgggtcaaaaaaaatataaaattcgactgtgaaacacattatgaaacttatggtaattctttatctgaaaaagttcaacttatgtcttcttgtaaacattttgttttatctaatagttcttttagttggtggacagaatttttatctatacgaggtgggattactatagcccccaaaaattggtatgcagatgaacgtgaagctgatatctatagaaaaaattggatttacttagaagataagacagaggaagagtaatgggatttctatttttaactataatacttattttgtgggggtatagttttaccaatataaaaataagcccttttagtattttattcatgagtttagggatcttttactctcaatttacttcaataaatattgacttaataataaaagtactttttttgataacttccataatttatcttattaaagataaatattcaaaaaaatacgttttttctttattattaattgcagtattaattttaattgagtcaactagtccctctaaatttaatcaatattatggttttattgatgctttgacatcatttgcaaccttctcaacaggcatacttctattttccataaaatttagtttacaagaacgcagaagtattttaaaatcaatttcatatttgccaatcttttcagtgttaattggaatccctctaacttttggtggttttatatctatgacagctagaggcggaattgccctttcaggagcagctttagaaacaaatttatcttttttttcagttctaagccttgtttcattagatattttatatcaggacactcgttctaataaatatcaaattttaaaaattattaactttatattgctatgttgtactttaacacgaggcggtattatttctggaattatcattattttaccaagtttactatttcttttaaaaaaaggatttaaaggagtaagacaatttatttttttgattattactatttttgggagtatttatccgcttattttattgtggaaaagtattagtgagaggactttcagtgcagatggtattaatacttcaggtcgatatacggcctgggactatattgtgaatttgacaacaaacaaatctcagggaatgggattgggaagtttaaagacattaactgaggatattaatttacgtgcctttactgctgctcataatacatatattcaattttattatgaaactggttatttgggagtaacactattatctattttatttattttaatattaataataatcctaaaattgactaattatagaaaaaaaatcatttacttaacattcatttcatttttagtatatagttatacagataattgtattgttaataatagatactggtatttgtttatgtttattataggatgttttaaatattttgacagaaaggaagaaaatgcgctactttaaaatattatttgagattattcaactattggtagctagtattttatgtagattatataaaaatccaaatgatatctggctaataaatgaaaaacctgatgaagctagagataatggttatgctttttatcaatatttaagaaagaatttccccgatattaaagtttattatgtaatcagtaaagagtctactgatatttataagtttgataatgaaactaacattgtattttataagagttttttacattttattttatatatcaaatctaaagttttaattagttctcaaacattgccctatccatctagcagaaaattatgtgaagcgctaatgtaccttaatttgaataaaccaaagaggatttggttacaacatggagttactaaagataaactcccatatgagaatatggcaagggaaatttttaagtatgatttaataacctgtgtttcattaaaagaggctaattttataatgaaagaatatggatataatgaagatcaggtgaaggctcttggatttgcaagatatgataatttgccaattggaaataataacacatttgatatacttataatgcctactttccgtaagggttacgagattaaaaattttagtctcccaacagatagtgaaactaaacattttgaggaaagtgtattctttaaaacatatgttgatttattgaattctgaagagctagacgagtatttagaaaagtctggtaaaaaagcaattttttatttacactatgcttttcaaccatatgcaaaatctttttctaaacgactaatgtcttcaaatgttatcattgctgaaagaacagaatatgatgttcaaaaactattaattaattgtgaattgctaattacagattattcaagcgttttttttgatttttcatatatgaaaaaacctgaaatatttttccattttgatgagaaagaatatagaagtaatcattatagggagggatattttgattataaaacagatggatttggtccagtagttaattctaaagaagaattactaactgaaatcaaagagtttattgataacccatctctgttaatggaatttaataagcgagctaataatttcttcaaatatactgataacaataattgccaacgtattttaaaagaaatttggagaattaatgaaactaattaagaattatttaatgacaagctcttatcaattgttaattattatcttaccaataataacaacaccatatatatcgagagtacttagtccagaagggattggactttattcatatacttatactattacacagtatttcgtattatttgctactcttggtactgttacgtatggtagcagagagatagcatattatcagtcaaataaacaaaagagaagtgaaattttttggggaattaccttccttagctgggctactggtgctatatcacttttaatattttatatatttattttttttaatggcaaatatagtgttttatttttttggcaaagctttttgatttttggagttatttttgatattaattggtatttcacaggaatggaaaagttcaaggttattatttcacgtaacttttgtataaaaattattagtttattgtgtatttttgtctttgtaaaatctgagaaagatttaagtttatatatagttatactaggattgagcaatataataggtaatatattagtttggccatatttgagaaaagaggtttataaacctaatttttctaagttatcattcaaaaaacatttgggaagtacatggatattttttttgccacaaacttctgttactttaaactcattaataaaccaaaatatgattgcatattttgactcaataacaagcttaggatactttacacaaacaaataagtttactgtgattgcgatttcaatagttatttcaattgggactgttatgttgcctagaatgtccaatttagttgcgcgcaaagagtattcaaagtttacagactatgttactaagagtattaatataagctcaggaatttctatagcaataatgtttggtttaatggctatagcacctaagtttacaactttttttttaggagctcaatataaatttgttattcatttgctagttttatcatcaccgatagtggttttagtaacctggagtaatgttcttggtcaacaatatttaatacctttaaataggatgaaaatatttacaaaatctctaatttgtggaaacttagtaaatgtttctctaaacttgattttgttacccaaaatgggagtagaaatttcaataataaatcagttaattaatgaaattattattgtaggtattcaatttatatcagttagaaaagagttaaaaataaatataatattaggagatctaataaaatatttttttgcgggtataattatgtttattgccgttttatatctgaatttacaattaccgatgactatcttcacactacttatagagattggtattggagttcttatatattcaatgctagttatttctcttaaaactggattatataaagaattgaaaaagattattaaaattcgttagcttaaaatctatcacctttcatttgagtagtaagaaatacaaagctttattataaaatttatcatttttaagactatcataaaagaagaaggatgacatggaacgaaaaaagaagaaaaaaaaaatatatataattattctaatattattaatgtttatcactattgtttgttttgggggatatgctacacgagagttaattactcccactgaaaaaacaataccaaatgtctcggatcaacctaaaaaaacttcggcctctaacggttatgtagagcaaaaaggggaagaagctgctgtgggtagtatagcacttgtagacgatgctggtgtaccagaatgggttaaagttccctcaaaggtaaatctagataaatttactgatttatctacgaataatatcactatttatcgaattaataatccggaagtcttaaaaacagttaccaatcgtacagatcaacggatgaaaatgtcagaagttatagctaagtatcctaatgctttgattatgaatgcttccgcttttgatatgcagacaggacaagtagctggatttcaaattaataatggaaagttgattcaagactggagtccaggtacaacgactcaatatgcttttgttattaacaaagatggttcgtgcaaaatttatgattcaagtacacctgctttaactattattaaaaatggagggcaacaagcctatgattttggtactgcgattatccgtgatggtaaaattcaaccaagtgatggctcagtagattggaagattcatatttttattgcgaatgataaagataataatctctatgctattttgagtgatacaaatgcaggttatgataatataataaaatcagtatcaaatttgaagctccaaaatatgttattacttgatagtggtggctcaagtcaactatctgtcaatggtaaaacgattgttgctagtcaagatgatcgagccgtaccggattatattgtgatgaaataaaaataaaagaacctcatggttcttttattttagagatttttcaaaaagggttttgactgagtctaattctgtttgagaaacgaccttagctccattttcatctgttgtatgtagattgagcttgctggtattctttagagccttactgtagctcataacgatcgttttagcatcattgaaacttatattggttttaacagtgtttgaaaaagcatagagaatttctcgatagtaattgaaactttcaagttttttcatatgagcaattttttgaatatttccgtagagttccattgagacattttgaatccgagtgattgaagcatccaaatcagtatcgtcaatttgtgtcatataggcttggacttgatcagcagtttgtaaattaacagttccttgtttaaactcataaccttcagccttgaatgcctttggattttgcatggtgattccaccagtggcctgtacaagtgatcccattttattaacatcgatctgaattactttgttaatggacgcattcaataggtctttaaccatctggaaaattccatcatctccattcgtattgtaaacttcagtgattgttttttgattaggcattgtcgcaaaaactgggaagttcatgaaagtagtttgatttgtctttacattcgttgaagctaaaacagtagcataagctgaatttttagaattatttttaccagttgcaatgataagtgtggtgaatgttttagctttttttaagtcaatacttgttgttttagggaaattttcatatgatgttgaaaaggttgattcaacatttctataagctacgtaggctatagaagcaacagaaataattactaatacaaaaataattgaaataacttttagtactgtgtattttttcttacgataatgacgcctctttttttgattcat

SEQ ID NO: 6 - protein, 105 aa, Lactococcus lactis
  33204_epsR
MNDLFYHRLKELVESSGKSANQIERELGYPRNSLNNYKLGGEPSGTRLIGLSEYFNVSPKYLMGISDEPNDSSAINLFKTLTQEEKKEMFIICQKWLFLEYQIEL

SEQ ID NO: 7 - protein, 139 aa, Lactococcus lactis
  33204_epsX
LEVFYSYNSRINNLSKADKGKEVVKNSSEKNQIDLTYKKYYKNLPKSVQNKIDDISSKNKEVTLTCIWQSDSVISEQFQQNLQKYYGNKFWNIKNITYNGETSEQLLAEKVQNQVLATNPDVVLYEAPLFNDNQYRLLG

SEQ ID NO: 8 - protein, 259 aa, Lactococcus lactis
  33204_epsC
MQETQEQTIDLRGIFKIIRKRLGLILFSALIVTILGSIYTFFIASPVYTASTQLVVKLPNSDNSAAYAGEVTGNIQMANTINQVIVSPVILDKVQSNLNLSDDSFQKQVTAANQTNSQVIMLTVKYSNPYIAKKIADETAKIFSSDAAKLLNVTNVNILSKAKAQTTPISPKPKLYLAISVIAGLVLGLAIALLKELFDNKINKEEDIEALGLTVLGVTSYDQMSDFNKNTNKNGTQSGTKSSPPSDHEVNRSSKRNKR

SEQ ID NO: 9 - protein, 231 aa, Lactococcus lactis
  33204_epsD
MAKNKRSIDNNHYIITSVNPQSPISEQYRTIRTTIDFKMADQGIKSFLVTSSETDEGKTTVSANIAVAFAQQGKKVLLIDGDLRKPTVNITFKVQNRVGLTNILMHQSSIEDAIQGTRLSENLTIITSGPIPPNPSELLASSAMKNLIDSVSDFFDVVLIDIPPLSAVTDAQILSSYVGGVVLVVRAYETKKESLAKTKKKLEQVNANILGVVLHGVDSSDSPSYYYYGVE

SEQ ID NO: 10 - protein, 254 aa, Lactococcus lactis
  33204_epsB
MIDIHCHILPGIDDGAKTSGDTLTMLKSAIDEGITTITATPHHNPQFNNESPLILKKVKEVQNIIDEHQLPIEVLPGQEVRIYGDLLKEFSEGKLLKAAGTSSYILIEFPSNHVPAYAKELFYNIKLEGLQPILVHPERNSGIIENPDILFDFIEQGVLSQITASSVTGHFGKKIQKLSFKMIENHLTHFVASDAHNVTSRAFKMKEAFEIIEDSYGSGVSRMLQNNADSVILNESFYQEEPIKIKTKKFLGLF

SEQ ID NO: 11 - protein, 226 aa, Lactococcus lactis
  33204_epsE
MEVFEASSELEEPKLVELKKFSRREIIIKRGIDILGGLAGSGLFLIAAALLYVPYKMSSKKDQGPMFYKQKRYGKNGKIFYILKFRTMIINAEQYLELHPEVKAAYHANGNKLESDPRVTKIGSFIRQHSIDELPQFINVLKGDMSLVGPRPILLFEAKEYGERLSYLLICKPGITGYWTTHGRSKVLFPQRADLELYYLQYHSTKNDIKLIMLTIKQILHGSDAY

SEQ ID NO: 12 - protein, 156 aa, Lactococcus lactis
  33204_GT1
MKKKTTKICMISSSGGHLKELNELIEISEQYETFQITEKDKFSNIKIGTRQYYVNKIDRDEKNFLFHFFILFLKIFQIFAVEKPKVIVTTGALVAYPACLIGKLMRAKVIFIESYARTETLSLTGKLVYRLSDLFIVQWPDLSKKYSKAKYYGELF

SEQ ID NO: 13 - protein, 160 aa, Lactococcus lactis
  33204_GT2
MILIILGTQKFQFNRLIKKVDKLIEDDQIKDSVIAQIGYSNYKPINYKFSDFFDQSEFDSLINKSDIIITHGGVGGIVSSLKKNKKIIVVPRLKKYREHIDDHQLEIARAFQRKNLVILNENLNELCNDISKIESFEPIHYVKDNKKIICEIKKFISKVK

SEQ ID NO: 14 - protein, 316 aa, Lactococcus lactis
  33204_GT3
MIKLSIIIPIYNVEKYLSKCLNSILEQTYKEIEIILVNDGSTDNSKDIAVSYCERFPNVFKYFEKDNGGLSSARNFGLEKISGDFVGFLDSDDYIDNDLYEIMINSLDSSIKIVECDFIWEYENGKSVLDKTSEYNSIKDLMVNGRVVAWNKIYNVEWLEKINIKFKEGLLYEDLNFFFKIVPHLTSISEVSTVKNSFVHYVQHKGTITSDNSLNILDIIKSYEDVFHYYNEKQINDLYFDELEYKFSRNLMGAFLKRAIKIKDKRQRKIILDEFWNNVLSYYPNWKKNKYIKKLSKQNILLFFINKYTYKLFYLL

SEQ ID NO: 15 - protein, 309 aa, Lactococcus lactis
  33204_GT4
MIYVEIRGNLGNQLFIYATAKKIQKLTGQKIQLNTTTLNKYFPNYKFGLSEFIMEDPDCFIESYKKLPWFTNEYLLPIKIFKKILNKTPKINKILSDFFFKAFEKKGYFIWRGETFKKFSLGNHKNYYLSGFWQSEEYFYDIRDELLEIITPINSIRECNFELLNLIRNSESICVSIRRGDYVDNPKISAIYNVCDINYFIESVNEIKKNVVNVKVICFSDDVEWVKKNIKFDCETHYETYGNSLSEKVQLMSSCKHFVLSNSSFSWWTEFLSIRGGITIAPKNWYADEREADIYRKNWIYLEDKTEEE

SEQ ID NO: 16 - protein, 396 aa, Lactococcus lactis
  33204_wzy
MGFLFLTIILILWGYSFTNIKISPFSILFMSLGIFYSQFTSINIDLIIKVLFLITSIIYLIKDKYSKKYVFSLLLIAVLILIESTSPSKFNQYYGFIDALTSFATFSTGILLFSIKFSLQERRSILKSISYLPIFSVLIGIPLTFGGFISMTARGGIALSGAALETNLSFFSVLSLVSLDILYQDTRSNKYQILKIINFILLCCTLTRGGIISGIIIILPSLLFLLKKGFKGVRQFIFLIITIFGSIYPLILLWKSISERTFSADGINTSGRYTAWDYIVNLTTNKSQGMGLGSLKTLTEDINLRAFTAAHNTYIQFYYETGYLGVTLLSILFILILIIILKLTNYRKKIIYLTFISFLVYSYTDNCIVNNRYWYLFMFIIGCFKYFDRKEENALL

SEQ ID NO: 17 - protein, 396 aa, Lactococcus lactis
  33204_Glyphos_trans
MRYFKILFEIIQLLVASILCRLYKNPNDIWLINEKPDEARDNGYAFYQYLRKNFPDIKVYYVISKESTDIYKFDNETNIVFYKSFLHFILYIKSKVLISSQTLPYPSSRKLCEALMYLNLNKPKRIWLQHGVTKDKLPYENMAREIFKYDLITCVSLKEANFIMKEYGYNEDQVKALGFARYDNLPIGNNNTFDILIMPTFRKGYEIKNFSLPTDSETKHFEESVFFKTYVDLLNSEELDEYLEKSGKKAIFYLHYAFQPYAKSFSKRLMSSNVIIAERTEYDVQKLLINCELLITDYSSVFFDFSYMKKPEIFFHFDEKEYRSNHYREGYFDYKTDGFGPVVNSKEELLTEIKEFIDNPSLLMEFNKRANNFFKYTDNNNCQRILKEIWRINETN

SEQ ID NO: 18 - protein, 472 aa, Lactococcus lactis
  33204_wzx
MKLIKNYLMTSSYQLLIIILPIITTPYISRVLSPEGIGLYSYTYTITQYFVLFATLGTVTYGSREIAYYQSNKQKRSEIFWGITFLSWATGAISLLIFYIFIFFNGKYSVLFFWQSFLIFGVIFDINWYFTGMEKFKVIISRNFCIKIISLLCIFVFVKSEKDLSLYIVILGLSNIIGNILVWPYLRKEVYKPNFSKLSFKKHLGSTWIFFLPQTSVTLNSLINQNMIAYFDSITSLGYFTQTNKFTVIAISIVISIGTVMLPRMSNLVARKEYSKFTDYVTKSINISSGISIAIMFGLMAIAPKFTTFFLGAQYKFVIHLLVLSSPIVVLVTWSNVLGQQYLIPLNRMKIFTKSLICGNLVNVSLNLILLPKMGVEISIINQLINEIIIVGIQFISVRKELKINIILGDLIKYFFAGIIMFIAVLYLNLQLPMTIFTLLIEIGIGVLIYSMLVISLKTGLYKELKKIIKIR

SEQ ID NO: 19 - protein, 299 aa, Lactococcus lactis
  33204_epsL
MERKKKKKKIYIIILILLMFITIVCFGGYATRELITPTEKTIPNVSDQPKKTSASNGYVEQKGEEAAVGSIALVDDAGVPEWVKVPSKVNLDKFTDLSTNNITIYRINNPEVLKTVTNRTDQRMKMSEVIAKYPNALIMNASAFDMQTGQVAGFQINNGKLIQDWSPGTTTQYAFVINKDGSCKIYDSSTPALTIIKNGGQQAYDFGTAIIRDGKIQPSDGSVDWKIHIFIANDKDNNLYAILSDTNAGYDNIIKSVSNLKLQNMLLLDSGGSSQLSVNGKTIVASQDDRAVPDYIVMK

SEQ ID NO: 20 - protein, 300 aa, Lactococcus lactis
  33204_lytR
MNQKKRRHYRKKKYTVLKVISIIFVLVIISVASIAYVAYRNVESTFSTSYENFPKTTSIDLKKAKTFTTLIIATGKNNSKNSAYATVLASTNVKTNQTTFMNFPVFATMPNQKTITEVYNTNGDDGIFQMVKDLLNASINKVIQIDVNKMGSLVQATGGITMQNPKAFKAEGYEFKQGTVNLQTADQVQAYMTQIDDTDLDASITRIQNVSMELYGNIQKIAHMKKLESFNYYREILYAFSNTVKTNISFNDAKTIVMSYSKALKNTSKLNLHTTDENGAKVVSQTELDSVKTLFEKSLK

SEQ ID NO: 21 - protein, 105 aa, Lactococcus lactis; identical to SEQ ID NO: 6
  33200_epsR

SEQ ID NO: 22 - protein, 139 aa, Lactococcus lactis; identical to SEQ ID NO: 7
  33200_epsX

SEQ ID NO: 23 - protein, 259 aa, Lactococcus lactis
  33200_epsC
MQETQEQTIDLRGIFKIIRKRLGLILFSALIVTILGSIYTFFIASPVYTASTQLVVKLPNSDNSAAYAVEVTGNIQMANTINQVIVSPVILDKVQSNLNLSDDSFQKQVTAANQTNSQVIMLTVKYSNPYIAKKIADETAKIFSSDAAKLLNVTNVNILSKAKAQTTPISPKPKLYLAISVIAGLVLGLAIALLKELFDNKINKEEDIEALGLTVLGVTSYDQMSDFNKNTNKNGTQSGTKSSPPSDHEVNRSSKRNKR

SEQ ID NO: 24 - protein, 231 aa, Lactococcus lactis; identical to SEQ ID NO: 9
  33200_epsD

SEQ ID NO: 25 - protein, 254 aa, Lactococcus lactis; identical to SEQ ID NO: 10
  33200_epsB

SEQ ID NO: 26 - protein, 226 aa, Lactococcus lactis; identical to SEQ ID NO: 11
  33200_epsE

SEQ ID NO: 27 - protein, 156 aa, Lactococcus lactis; identical to SEQ ID NO: 12
  33200_GT1

SEQ ID NO: 28 - protein, 160 aa, Lactococcus lactis; identical to SEQ ID NO: 13
  33200_GT2

SEQ ID NO: 29 - protein, 316 aa, Lactococcus lactis; identical to SEQ ID NO: 14
  33200_GT3

SEQ ID NO: 30 - protein, 309 aa, Lactococcus lactis; identical to SEQ ID NO: 15
  33200_GT4

SEQ ID NO: 31 - protein, 396 aa, Lactococcus lactis; identical to SEQ ID NO: 16
  33200_wzy

SEQ ID NO: 32 - protein, 396 aa, Lactococcus lactis; identical to SEQ ID NO: 17
  33200_Glyphos_trans

SEQ ID NO: 33 - protein, 472 aa, Lactococcus lactis; identical to SEQ ID NO: 18
  33200_wzx

SEQ ID NO: 34 - protein, 299 aa, Lactococcus lactis; identical to SEQ ID NO: 19
  33200_epsL

SEQ ID NO: 35 - protein, 300 aa, Lactococcus lactis; identical to SEQ ID NO: 20
  33200_lytR

SEQ ID NO: 36 - protein, 105 aa, Lactococcus lactis; identical to SEQ ID NO: 6
  33222_epsR

SEQ ID NO: 37 - protein, 139 aa, Lactococcus lactis; identical to SEQ ID NO: 7
  33222_epsX

SEQ ID NO: 38 - protein, 259 aa, Lactococcus lactis; identical to SEQ ID NO: 8
  33222_epsC

SEQ ID NO: 39 - protein, 231 aa, Lactococcus lactis; identical to SEQ ID NO: 9
  33222_epsD

SEQ ID NO: 40 - protein, 254 aa, Lactococcus lactis; identical to SEQ ID NO: 10
  33222_epsB

SEQ ID NO: 41 - protein, 226 aa, Lactococcus lactis; identical to SEQ ID NO: 11
  33222_epsE

SEQ ID NO: 42 - protein, 156 aa, Lactococcus lactis; identical to SEQ ID NO: 12
  33222_GT1

SEQ ID NO: 43 - protein, 160 aa, Lactococcus lactis; identical to SEQ ID NO: 13
  33222_GT2

SEQ ID NO: 44 - protein, 316 aa, Lactococcus lactis; identical to SEQ ID NO: 14
  33222_GT3

SEQ ID NO: 45 - protein, 309 aa, Lactococcus lactis
  33222_GT4
MIYVEIRGNLGNQLFIYATAKKIQKLTGQKIQLNTTTLNKYFPNYKFGLSEFIMEDPDCFIESYKKLPWFTNEYLLPIKIFKKILNKTPKINKILSDFFFKAFEKKGYFIWRGETFKKFSLGNHKNYYLSGFWQSEEYFYDIRDELLEIITPINSIRECNFELLNLISNSESICVSIRRGDYVDNPKISAIYNVCDINYFIESVNEIKKNVVNVKVICFSDDVEWVKKNIKFDCETHYETYGNSLSEKVQLMSSCKHFVLSNSSFSWWTEFLSIRGGITIAPKNWYADEREADIYRKNWIYLEDKTEEE

SEQ ID NO: 46 - protein, 396 aa, Lactococcus lactis; identical to SEQ ID NO: 16
  33222_wzy

SEQ ID NO: 47 - protein, 396 aa, Lactococcus lactis; identical to SEQ ID NO: 17
  33222_Glyphos_trans

SEQ ID NO: 48 - protein, 472 aa, Lactococcus lactis; identical to SEQ ID NO: 18
  33222_wzx

SEQ ID NO: 49 - protein, 299 aa, Lactococcus lactis; identical to SEQ ID NO: 19
  33222_epsL

SEQ ID NO: 50 - protein, 300 aa, Lactococcus lactis; identical to SEQ ID NO: 20
  33222_lytR

SEQ ID NO: 51 - protein, 105 aa, Lactococcus lactis
  33225_epsR
MNDLFYHRLKELVESSGKSANQIERELGYPRNSLNNYKLGGEPSGTRLIGQSEYFNVSPKYLMGISDEPNDSSAINLFKTLTQEEKKEMFIICQKWLFLEYQIEL

SEQ ID NO: 52 - protein, 139 aa, Lactococcus lactis; identical to SEQ ID NO: 7
  33225_epsX

SEQ ID NO: 53 - protein, 259 aa, Lactococcus lactis; identical to SEQ ID NO: 8
  33225_epsC

SEQ ID NO: 54 - protein, 231 aa, Lactococcus lactis; identical to SEQ ID NO: 9
  33225_epsD

SEQ ID NO: 55 - protein, 254 aa, Lactococcus lactis; identical to SEQ ID NO: 10
  33225_epsB

SEQ ID NO: 56 - protein, 226 aa, Lactococcus lactis; identical to SEQ ID NO: 11
  33225_epsE

SEQ ID NO: 57 - protein, 156 aa, Lactococcus lactis; identical to SEQ ID NO: 12
  33225_GT1

SEQ ID NO: 58 - protein, 160 aa, Lactococcus lactis; identical to SEQ ID NO: 13
  33225_GT2

SEQ ID NO: 59 - protein, 316 aa, Lactococcus lactis; identical to SEQ ID NO: 14
  33225_GT3

SEQ ID NO: 60 - protein, 309 aa, Lactococcus lactis; identical to SEQ ID NO: 15
  33225_GT4

SEQ ID NO: 61 - protein, 396 aa, Lactococcus lactis; identical to SEQ ID NO: 16
  33225_wzy

SEQ ID NO: 62 - protein, 396 aa, Lactococcus lactis; identical to SEQ ID NO: 17
  33225_Glyphos_trans

SEQ ID NO: 63 - protein, 472 aa, Lactococcus lactis; identical to SEQ ID NO: 18
  33225_wzx

SEQ ID NO: 64 - protein, 299 aa, Lactococcus lactis; identical to SEQ ID NO: 19
  33225_epsL

SEQ ID NO: 65 - protein, 300 aa, Lactococcus lactis; identical to SEQ ID NO: 20
  33225_lytR

SEQ ID NO: 66 - protein, 105 aa, Lactococcus lactis; identical to SEQ ID NO: 6
  33133_epsR

SEQ ID NO: 67 - protein, 139 aa, Lactococcus lactis; identical to SEQ ID NO: 7
  33133_epsX

SEQ ID NO: 68 - protein, 259 aa, Lactococcus lactis
  33133_epsC
MQETQEQTIDLRGIFKIIRKRLGLILFSALIVTILGSIYTFFIASPVYTASTQLVVKLPNSDNSAAYAGEVTGNIQMANTINQVIVSPVILGKVQSNLNLSDDSFQKQVTAANQTNSQVITLTVKYSNPYIAKKIADETAKIFSSDAAKLLNVTNVNILSKAKAQTTPISPKPKLYLAISVIAGLVLGLAIALLKELFDNKINKEEDIEALGLTVLGVTSYDQMSDFNKNTNKNGTQSGTKSSPPSDHEVNRSSKRNKR

SEQ ID NO: 69 - protein, 231 aa, Lactococcus lactis
  33133_epsD
MAKNKRSIDNNHYIITSVNPQSPISEQYRTIRTTIDFKMADQGIKSFLVTSSETDEGKTAVSANIAVAFAQQGKKVLLIDGDLRKPTVNITFKVQNRVGLTNILMHQSSIEDAIQGTRLSENLTIITSGPIPPNPSELLASSAMKNLIDSVSDFFDVVLIDTPPLSAVTDAQILSSYVGGVVLVVRAYETKKESLAKTKKKLEQVNANILGVVLHGVDSSDSPSYYYYGVE

SEQ ID NO: 70 - protein, 254 aa, Lactococcus lactis; identical to SEQ ID NO: 10
  33133_epsB

SEQ ID NO: 71 - protein, 226 aa, Lactococcus lactis; identical to SEQ ID NO: 11
  33133_epsE

SEQ ID NO: 72 - protein, 156 aa, Lactococcus lactis; identical to SEQ ID NO: 12
  33133_GT1

SEQ ID NO: 73 - protein, 160 aa, Lactococcus lactis; identical to SEQ ID NO: 13
  33133_GT2

SEQ ID NO: 74 - protein, 316 aa, Lactococcus lactis; identical to SEQ ID NO: 14
  33133_GT3

SEQ ID NO: 75 - protein, 309 aa, Lactococcus lactis; identical to SEQ ID NO: 15
  33133_GT4

SEQ ID NO: 76 - protein, 396 aa, Lactococcus lactis; identical to SEQ ID NO: 16
  33133_wzy

SEQ ID NO: 77 - protein, 396 aa, Lactococcus lactis; identical to SEQ ID NO: 17
  33133_Glyphos_trans

SEQ ID NO: 78 - protein, 472 aa, Lactococcus lactis; identical to SEQ ID NO: 18
  33133_wzx

SEQ ID NO: 79 - protein, 299 aa, Lactococcus lactis; identical to SEQ ID NO: 19
  33133_epsL

SEQ ID NO: 80 - protein, 300 aa, Lactococcus lactis; identical to SEQ ID NO: 20
  33133_lytR

(19) United States
(12) Patent Application Publication
(10) Pub. No.: US 2022/0287318 A1
(43) Pub. Date: Sep. 15, 2022

(54) TEXTURING L. LACTIS WITH NIZO B40-LIKE EPS GENE CLUSTERS

(71) Applicant: Chr. Hansen A/S, Hoersholm (DK)

(72) Inventors: Vera Kuzina POULSEN, Hoersholm (DK); Gunnar OEREGAARD, Hoersholm (DK); Elahe Ghanei MOGHADAM, Hoersholm (DK)

(73) Assignee: Chr. Hansen A/S, Hoersholm (DK)

(21) Appl. No.: 17/634,938

(22) PCT Filed: Aug. 21, 2020
(86) PCT No.: PCT/EP2020/073535

§ 371 Date: Feb. 11, 2022

(30) Foreign Application Priority Data
Aug. 23, 2019 (EP) 19193318.3
Aug. 23, 2019 (EP) 19193319.1
Aug. 23, 2019 (EP) 19193321.7
Aug. 23, 2019 (EP) 19193323.3
Aug. 23, 2019 (EP) 19193335.7
Aug. 23, 2019 (EP) 19193336.5

(51) Int. Cl. A23C 9/123; C12N 1/20

(52) U.S. Cl. CPC A23C 9/1236; C12N 1/205; A23C 2220/202; A23C 2220/206

(57) ABSTRACT

The present invention provides novel Lactococcus lactis lactic acid bacterium strains having improved texturing properties and methods of using the strains for producing food products.

### FIELD OF THE INVENTION

The present invention relates to novel Lactococcus lactis lactic acid bacterium strains, having improved texturing properties. The present invention also relates to methods of using the strains for making food products and to food products comprising the strains.

### BACKGROUND OF THE INVENTION

Lactic acid bacteria (LAB) are used extensively by the food industry for fermentation of food. Conversion of fresh milk to fermented milk by LAB is a way of extending the life time of the milk and provides taste as well as texture.

Thus, important features of the strains used for milk fermentation include fast acidification, stable (no/low) post-acidification, long shelf-life and good texture. Good texture is typically high mouth thickness and viscosity (measured as high shear stress using a rheometer) and high gel firmness.

Some LAB strains contribute significantly to an improved texture associated with their ability to produce exo- (or extracellular) polysaccharides (EPS), which can be capsular (remain attached to the cell in the form of capsules) or secreted into the media. EPS consists of either a single type of sugar (homo-exopolysaccharides) or repeating units made of different sugars (hetero-exopolysaccharides). EPS-producing LAB are of interest, since EPS act as natural viscosifiers and texture enhancers of fermented foods. Furthermore, EPS from food-grade LAB with defined rheological properties have potential for development and exploitation as food additives. EPS are known to improve the rheological properties of LAB-fermented products by influencing viscosity, syneresis, firmness and sensory properties. The primary structural features (monosaccharide type and configuration, glycosidic linkage, non-sugar decorations, charge), the conformation and molecular weight, the amount of polysaccharide and the interactions of the polysaccharide with other system components are all factors that can contribute to and influence the displayed techno-functional properties (Zeidan et al., 2017).

Fermented milk can be produced by mesophilic LAB, e.g. Lactococcus sp. leading to, e.g., sour milk, or thermophilic LAB, e.g., Streptococcus thermophilus and Lactobacillus delbruckii subsp. bulgaricus, for yoghurt. Dairy products, such as fresh cheese, butter milk, sour milk and sour cream, prepared with mesophilic starter cultures, such as Lactococcus lactis, are in popular demand with consumers. In addition, market for dairy alternative products, where plant bases fermented with L. lactis can play a role, is growing. Consumers with lactose intolerance and milk allergy, as well as consumers with concerns about cow milk hormones and cholesterol, animal well-being and impact of animal-based food on the environment play a role in the increasing demand. Also, plant-based diet is supposedly healthier than meat-based diet (Tangyu et al., 2019).

WO 2017/108679 relates to the novel strain Lactococcus lactis subsp. lactis DSM 29291, which had the highest shear stress out of the eight different L. lactis subsp. lactis strains tested, both according to the TADM and the rheometer measurements (see Example 1 and FIG. 1 of WO 2017/108679).

Since mesophilic cultures are used for fermented milk products, and texture is an important parameter, there is a need for further texturing mesophilic strains, in particular for improved texturing mesophilic strains, e.g., texturing Lactococcus lactis strains.

### SUMMARY OF THE INVENTION

In a first aspect, the present invention relates to a Lactococcus lactis lactic acid bacterium (LAB) strain comprising an active eps gene cluster capable of producing exopolysaccharide (EPS), wherein the eps gene cluster is selected from:
(i) a nucleotide sequence as defined in SEQ ID NO.: 1;
(ii) a nucleotide sequence as defined in SEQ ID NO.: 2, or a nucleotide sequence which differs by no more than 1 nucleotide from the nucleotide sequence as defined in SEQ ID NO.: 2;
(iii) a nucleotide sequence as defined in SEQ ID NO.: 3, or a nucleotide sequence which differs by no more than 1 nucleotide from the nucleotide sequence as defined in SEQ ID NO.: 3;
(iv) a nucleotide sequence as defined in SEQ ID NO.: 4, or a nucleotide sequence which differs by no more than 1 nucleotide from the nucleotide sequence as defined in SEQ ID NO.: 4; and
(v) a nucleotide sequence as defined in SEQ ID NO.: 5, or a nucleotide sequence which differs by no more than 5 nucleotides from the nucleotide sequence as defined in SEQ ID NO.: 5,
   wherein the Lactococcus lactis LAB strain comprising the active eps gene cluster as defined in SEQ ID NO.: 1 belongs to the MLST (multilocus sequence typing) group ST76, wherein the MLST analysis is performed as described in Example 4 of the present description, i.e., with a 12 gene MLST scheme developed at Chr. Hansen. The scheme is based on the twelve genes dnaK, fusA, groEL, gyrA, gyrB, ileS, lepA, pheS, recA, rpoA, rpoB and rpoC chosen from the core genome of Lactobacillaceae (Salvetti et al., 2018). A total of 22493 bp are used in the scheme, which thereby represents almost 1% of the average Lactococcus genome. MLST typing with Illumina whole genome sequences is performed with the help of the CLC Microbial Genomics Module, which is a plugin to the CLC Genomics Workbench v10. In CLC, MLST is incorporated into Chr. Hansen's custom designed standard genome sequence analysis pipeline. It is performed both on de novo contigs and reference assemblies.

In a second aspect, the present invention relates to a composition comprising at least one lactic acid bacterium strain according to the present invention, as described above.

Preferably, the composition of the present invention comprises at least one lactic acid bacterium strain according to the present invention, as described above, and at least one further lactic acid bacterium strain (also referred to as “helper strain” or “co-acidifier strain”) comprising an active eps gene cluster capable of producing exopolysaccharide (EPS), wherein the further lactic acid bacterium strain (“helper strain” or “co-acidifier strain”) is able to (i) generate fermented milks with a pH of about 4.55 in about 15 h or less, preferably in about 12 h or less and is able to (ii) generate fermented milks having a shear stress of 40 Pa or more measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 ((i), time to pH 4.55) followed by storage at 4° C. until shear stress is measured, typically from 1-7 days, such as for 5 days, followed by gently stirring and measuring the shear stress at shear rate 300 s⁻¹ ((ii), shear stress), wherein the inoculation temperature is 30° C.

More preferably, the composition according to the second aspect of the present invention comprises at least one Lactococcus lactis lactic acid bacterium strain according to the present invention in combination with (a) a lactic acid bacterium strain comprising an active eps gene cluster capable of producing exopolysaccharide (EPS), wherein the eps gene cluster comprises the nucleotide sequences (a), (b) and (c) (c1 to c4) as defined in (vi), or (b) a lactic acid bacterium strain Lactococcus lactis comprising an active eps gene cluster capable of producing exopolysaccharide (EPS), wherein the eps gene cluster is as defined in (vii):
(vi) (a): a nucleotide sequence encoding a polypeptide having polymerase activity and having at least 95% identity with the amino acid sequence encoded by nucleotides 6955-8145 of SEQ ID NO: 1 (herein termed wzy);
   (b): a nucleotide sequence encoding a polypeptide having polysaccharide transporter activity and having at least 95% identity with the amino acid sequence encoded by nucleotides 9309-10727 of SEQ ID NO: 1 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 4008-4478 of SEQ ID NO: 1 (herein termed GT1);
   (c2): a nucleotide sequence having at least 95% identity with the amino acid sequence encoded by nucleotides 4478-4960 of SEQ ID NO: 1 (herein termed GT2);
   (c3): a nucleotide sequence having at least 95% identity with the amino acid sequence encoded by nucleotides 5015-5965 of SEQ ID NO: 1 (herein termed GT3); and
   (c4): a nucleotide sequence having at least 95% identity with the amino acid sequence encoded by nucleotides 6026-6955 of SEQ ID NO: 1 (herein termed GT4);
(vii) a nucleotide sequence as defined in SEQ ID NO.: 5.

The composition of the present invention may comprise further components, such as cryoprotectants, lyoprotectants, antioxidants, nutrients, fillers, flavorants or mixtures thereof, as described in detail below.

In a third aspect, the present invention relates to the use of the lactic acid bacterium strains of the present invention and/or the composition of the present invention for increasing the viscosity (measured as shear stress with a shear rate of 300 s⁻¹, as described in the present invention) of a fermented milk product. The third aspect further relates to the use of the Lactococcus lactis subsp. cremoris strain DSM 25485 and/or the Lactococcus lactis subsp. lactis strain DSM 33192 for increasing viscosity (measured as shear stress with a shear rate of 300 s⁻¹, as described in the present invention) of a fermented milk product. The fermented milk product may be a mammalian-based fermented milk product (i.e., the milk base which is fermented has mammalian origin) or a plant-based fermented milk product (i.e., the milk base which is fermented is derived from plants, such as soy milk).

In a fourth aspect, the invention relates to a method of producing a food product comprising at least one stage in which at least one lactic acid bacterium strain as defined in the first aspect of the present invention, and/or the composition as defined in the second aspect of the present invention is used. The present invention further relates to a food product comprising at least one lactic acid bacterium strain as defined in the first aspect of the present invention, and/or the composition as defined in the second aspect of the present invention. The food product may comprise further components, such as thickeners or stabilizers, or mixtures thereof, as described in detail below.

In a fifth aspect, the present invention relates to a method for manufacturing a Lactococcus lactis lactic acid bacterium (LAB) strain which comprises the following steps:
(a) providing a Lactococcus lactis lactic acid bacterium (LAB) strain comprising an active eps gene cluster capable of producing exopolysaccharide (EPS), wherein the eps gene cluster is selected from:
(i) a nucleotide sequence as defined in SEQ ID NO.: 1;
(ii) a nucleotide sequence as defined in SEQ ID NO.: 2, or a nucleotide sequence which differs by no more than 1 nucleotide from the nucleotide sequence as defined in SEQ ID NO.: 2;
(iii) a nucleotide sequence as defined in SEQ ID NO.: 3, or a nucleotide sequence which differs by no more than 1 nucleotide from the nucleotide sequence as defined in SEQ ID NO.: 3;
(iv) a nucleotide sequence as defined in SEQ ID NO.: 4, or a nucleotide sequence which differs by no more than 1 nucleotide from the nucleotide sequence as defined in SEQ ID NO.: 4; and
(v) a nucleotide sequence as defined in SEQ ID NO.: 5, or a nucleotide sequence which differs by no more than 5 nucleotides from the nucleotide sequence as defined in SEQ ID NO.: 5;
(b) screening for a Lactococcus lactis lactic acid bacterium (LAB) strain which is
   (i) able to generate fermented milks having a shear stress of 40 Pa or more, preferably 50 Pa or more, more preferably 55 Pa or more, even more preferably more than 56 Pa, such as 60 Pa, 61 Pa, 62 Pa, 63 Pa, 64 Pa, 65 Pa, 66 Pa, 67 Pa, 68 Pa, 69 Pa, 70 Pa, 75 Pa, 80 Pa, 85 Pa, 86 Pa, 87 Pa, 88 Pa, 89 Pa, 90 Pa, 95 Pa, 98 Pa, 100 Pa, 105 Pa, 110 Pa, 115 Pa, 120 Pa or 121 Pa or more, measured at shear rate 300 s⁻¹, measured under following conditions:
200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C.; and/or
(ii) able to generate fermented milks having a shear stress greater than 24 Pa, such as about 35 Pa, 36 Pa, 45 Pa, 47 Pa, 54 Pa, 56 Pa, 57 Pa, 60 Pa, 62 Pa, 63 Pa, 64 Pa, 71 Pa, 74 Pa, 75 Pa, 79 Pa, 86 Pa, 88 Pa, 93 Pa, 96 Pa, 99 Pa, 102 Pa, 106 Pa or more, measured at shear rate 300 s⁻¹, under the following conditions: 200 ml of soy milk supplemented with 2% glucose (as described in Example 2) are inoculated with 2 ml of an overnight culture of the lactic acid bacterium strain, and left at inoculation temperature until pH^{˜}4.55 (such as pH 4.49, 4.53, 4.54, 4.55 or 4.66), followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

### BRIEF DESCRIPTION OF THE FIGURES

FIG. 1: Eps gene cluster in L. lactis cremoris strain B40 (pNZ4000, GenBank AF036485). The nomenclature used is the one suggested by Zeidan et al. (2017).

FIG. 2: Display of the differences between the strains. ClustVis, a web tool for visualizing clustering of multivariate data (https://biit.cs.ut.ee/clustvis/) was used for the analysis, using standard parameters. Milk acidification data (minimal pH, Vmax), the ability of the strains to grow on different carbon sources, texturing ability, phage resistance and nisin sensitivity data were to make the plots. (A) PCA method “SVD with imputation” is used to calculate principal components. Unit variance scaling is applied to rows. X and Y axis show principal component 1 and principal component 2 that explain 43.2% and 21.3% of the total variance, respectively. N=21 data points representing the strains. (B) For the heatmap construction, rows were centered; unit variance scaling was applied to rows. Both rows and columns were clustered using correlation distance and average linkage. 25 rows represent the different experiments, 21 columns represent the strains.

FIG. 3: Experimental setup for characterization of strains for their texturing, milk acidification properties, and media (e.g.; C source) preferences in 96-well micro-titer plate format. Milk added pH indicator was fermented in micro-titer plates, which were incubated on flat-bed scanners at 30° C.; plate bottom was scanned every 6 min, and the color development was correlated with pH changes in milk (see Poulsen et al., 2019 for details). Strain texturing properties were assessed using TADM tool of the liquid handling robot Hamilton, where pressure versus time curves obtained during pipetting of the samples were used to assess the relative shear stress of the samples. Samples with the highest relative shear stress were represented by aspiration curves with the lowest pressure values (Pa) or highest curve area (see Poulsen et al., 2019 for details). For selected samples, milk acidification until pH 4.55 and shear stress measurements are performed in 200-ml scale using rheometer. Milk pH is measured by an electrode, and the shear stress of fermented milk is measured using rheometer.

### BRIEF DESCRIPTION OF THE SEQUENCE LISTING

SEQ ID NO:1 sets out the complete sequence of the eps gene cluster of Lactococcus lactis strains DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192.

SEQ ID NO:2 sets out the complete sequence of the eps gene cluster of Lactococcus lactis strains DSM 33200, DSM 33201, DSM 33202 and DSM 33203.

SEQ ID NO:3 sets out the complete sequence of the eps gene cluster of Lactococcus lactis strain DSM 33222.

SEQ ID NO:4 sets out the complete sequence of the eps gene cluster of Lactococcus lactis strain DSM 33225.

SEQ ID NO:5 sets out the complete sequence of the eps gene cluster of Lactococcus lactis strain DSM 33133.

SEQ ID NO:6 sets out the amino acid sequence encoded by the epsR gene of DSM 33204, DSM 33205, DSM 33220DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 1-318 of SEQ ID NO.: 1);

SEQ ID NO:7 sets out the amino acid sequence encoded by the epsXgene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 407-826 of SEQ ID NO.: 1);

SEQ ID NO:8 sets out the amino acid sequence encoded by the epsCgene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 993-1772 of SEQ ID NO.: 1);

SEQ ID NO:9 sets out the amino acid sequence encoded by the epsD gene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 1782-2477 of SEQ ID NO.: 1);

SEQ ID NO:10 sets out the amino acid sequence encoded by the epsB gene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 2532-3296 of SEQ ID NO.: 1);

SEQ ID NO:11 sets out the amino acid sequence encoded by the epsE gene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 3318-3998 of SEQ ID NO.: 1);

SEQ ID NO:12 sets out the amino acid sequence of a putative glycosyltransferase (GT1) of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 4008-4478 of SEQ ID NO.: 1);

SEQ ID NO:13 sets out the amino acid sequence of a putative glycosyltransferase (GT2) of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 4478-4960 of SEQ ID NO.: 1);

SEQ ID NO:14 sets out the amino acid sequence of a putative glycosyltransferase (GT3) of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 5015-5965 of SEQ ID NO.: 1);

SEQ ID NO:15 sets out the amino acid sequence of a putative glycosyltransferase (GT4) of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 6026-6955 of SEQ ID NO.: 1);

SEQ ID NO:16 sets out the amino acid sequence encoded by the wzy gene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 6955-8145 of SEQ ID NO.: 1);

SEQ ID NO:17 sets out the amino acid sequence of a putative glycerophosphotransferase (glyphos trans) family protein of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 8132-9322 of SEQ ID NO.: 1);

SEQ ID NO:18 sets out the amino acid sequence encoded by the wzx gene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 9309-10727 of SEQ ID NO.: 1);

SEQ ID NO:19 sets out the amino acid sequence encoded by the epsL gene of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 10825-11724 of SEQ ID NO.: 1);

SEQ ID NO:20 sets out the amino acid sequence of the IytR protein of DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193 and DSM 33192 (nucleotides 11749-12651 of the complementary strand of SEQ ID NO.: 1);

SEQ ID NO:21 sets out the amino acid sequence encoded by the epsR gene of DSM 33200, DSM 33201, DSM 33202 and DSM 33203 (nucleotides 1-318 of SEQ ID NO.: 2);

SEQ ID NO:22 sets out the amino acid sequence encoded by the epsX gene of DSM 33200, DSM 33201, DSM 33202 and DSM 33203 (nucleotides 407-826 of SEQ ID NO.: 2);

SEQ ID NO:23 sets out the amino acid sequence encoded by the epsC gene of DSM 33200, DSM 33201, DSM 33202 and DSM 33203 (nucleotides 993-1772 of SEQ ID NO.: 2);

SEQ ID NO:24 sets out the amino acid sequence encoded by the epsD gene of DSM 33200, DSM 33201, DSM 33202 and DSM 33203 (nucleotides 1782-2477 of SEQ ID NO.: 2);

SEQ ID NO:25 sets out the amino acid sequence encoded by the epsB gene of DSM 33200, DSM 33201, DSM 33202 and DSM 33203 (nucleotides 2532-3296 of SEQ ID NO.: 2);

SEQ ID NO:26 sets out the amino acid sequence encoded by the epsE gene of DSM 33200, DSM 33201, DSM 33202 and DSM 33203 (nucleotides 3318-3998 of SEQ ID NO.: 2);

SEQ ID NO:27 sets out the amino acid sequence of a putative glycosyltransferase (GT1) of DSM 33200, DSM 33201, DSM 33202 and DSM 33203 (nucleotides 4008-4478 of SEQ ID NO.: 2);

SEQ ID NO:28 sets out the amino acid sequence of a putative glycosyltransferase (GT2) of DSM 33200, DSM 33201, DSM 33202 and DSM 33203 (nucleotides 4478-4960 of SEQ ID NO.: 2);

SEQ ID NO:29 sets out the amino acid sequence of a putative glycosyltransferase (GT3) of DSM 33200, DSM 33201, DSM 33202 and DSM 33203 (nucleotides 5015-5965 of SEQ ID NO.: 2);

SEQ ID NO:30 sets out the amino acid sequence of a putative glycosyltransferase (GT4) of DSM 33200, DSM 33201, DSM 33202 and DSM 33203 (nucleotides 6026-6955 of SEQ ID NO.: 2);

SEQ ID NO:31 sets out the amino acid sequence encoded by the wzy gene of DSM 33200, DSM 33201, DSM 33202 and DSM 33203 (nucleotides 6955-8145 of SEQ ID NO.: 2);

SEQ ID NO:32 sets out the amino acid sequence of a putative glycerophosphotransferase (glyphos trans) family protein of DSM 33200, DSM 33201, DSM 33202 and DSM 33203 (nucleotides 8132-9322 of SEQ ID NO.: 2);

SEQ ID NO:33 sets out the amino acid sequence encoded by the wzx gene of DSM 33200, DSM 33201, DSM 33202 and DSM 33203 (nucleotides 9309-10727 of SEQ ID NO.: 2);

SEQ ID NO:34 sets out the amino acid sequence encoded by the epsL gene of DSM 33200, DSM 33201, DSM 33202 and DSM 33203 (nucleotides 10825-11724 of SEQ ID NO.: 2);

SEQ ID NO:35 sets out the amino acid sequence of the lytR protein of DSM 33200, DSM 33201, DSM 33202 and DSM 33203 (nucleotides 11749-12651 of the complementary strand of SEQ ID NO.: 2);

SEQ ID NO:36 sets out the amino acid sequence encoded by the epsR gene of DSM 33222 (nucleotides 1-318 of SEQ ID NO.: 3);

SEQ ID NO:37 sets out the amino acid sequence encoded by the epsX gene of DSM 33222 (nucleotides 407-826 of SEQ ID NO.: 3);

SEQ ID NO:38 sets out the amino acid sequence encoded by the epsC gene of DSM 33222 (nucleotides 993-1772 of SEQ ID NO.: 3);

SEQ ID NO:39 sets out the amino acid sequence encoded by the epsD gene of DSM 33222 (nucleotides 1782-2477 of SEQ ID NO.: 3);

SEQ ID NO:40 sets out the amino acid sequence encoded by the epsB gene of DSM 33222 (nucleotides 2532-3296 of SEQ ID NO.: 3);

SEQ ID NO:41 sets out the amino acid sequence encoded by the epsE gene of DSM 33222 (nucleotides 3318-3998 of SEQ ID NO.: 3);

SEQ ID NO:42 sets out the amino acid sequence of a putative glycosyltransferase (GT1) of DSM 33222 (nucleotides 4008-4478 of SEQ ID NO.: 3);

SEQ ID NO:43 sets out the amino acid sequence of a putative glycosyltransferase (GT2) of DSM 33222 (nucleotides 4478-4960 of SEQ ID NO.: 3);

SEQ ID NO:44 sets out the amino acid sequence of a putative glycosyltransferase (GT3) of DSM 33222 (nucleotides 5015-5965 of SEQ ID NO.: 3);

SEQ ID NO:45 sets out the amino acid sequence of a putative glycosyltransferase (GT4) of DSM 33222 (nucleotides 6026-6955 of SEQ ID NO.: 3);

SEQ ID NO:46 sets out the amino acid sequence encoded by the wzy gene of DSM 33222 (nucleotides 6955-8145 of SEQ ID NO.: 3);

SEQ ID NO:47 sets out the amino acid sequence of a putative glycerophosphotransferase (glyphos trans) family protein of DSM 33222 (nucleotides 8132-9322 of SEQ ID NO.: 3);

SEQ ID NO:48 sets out the amino acid sequence encoded by the wzx gene of DSM 33222 (nucleotides 9309-10727 of SEQ ID NO.: 3);

SEQ ID NO:49 sets out the amino acid sequence encoded by the epsL gene of DSM 33222 (nucleotides 10825-11724 of SEQ ID NO.: 3);

SEQ ID NO:50 sets out the amino acid sequence of the lytR protein of DSM 33222 (nucleotides 11749-12651 of the complementary strand of SEQ ID NO.: 3);

SEQ ID NO:51 sets out the amino acid sequence encoded by the epsR gene of DSM 33225 (nucleotides 1-318 of SEQ ID NO.: 4);

SEQ ID NO:52 sets out the amino acid sequence encoded by the epsX gene of DSM 33225 (nucleotides 407-826 of SEQ ID NO.: 4);

SEQ ID NO:53 sets out the amino acid sequence encoded by the epsC gene of DSM 33225 (nucleotides 993-1772 of SEQ ID NO.: 4);

SEQ ID NO:54 sets out the amino acid sequence encoded by the epsD gene of DSM 33225 (nucleotides 1782-2477 of SEQ ID NO.: 4);

SEQ ID NO:55 sets out the amino acid sequence encoded by the epsB gene of DSM 33225 (nucleotides 2532-3296 of SEQ ID NO.: 4);

SEQ ID NO:56 sets out the amino acid sequence encoded by the epsE gene of DSM 33225 (nucleotides 3318-3998 of SEQ ID NO.: 4);

SEQ ID NO:57 sets out the amino acid sequence of a putative glycosyltransferase (GT1) of DSM 33225 (nucleotides 4008-4478 of SEQ ID NO.: 4);

SEQ ID NO:58 sets out the amino acid sequence of a putative glycosyltransferase (GT2) of DSM 33225 (nucleotides 4478-4960 of SEQ ID NO.: 4);

SEQ ID NO:59 sets out the amino acid sequence of a putative glycosyltransferase (GT3) of DSM 33225 (nucleotides 5015-5965 of SEQ ID NO.: 4);

SEQ ID NO:60 sets out the amino acid sequence of a putative glycosyltransferase (GT4) of DSM 33225 (nucleotides 6026-6955 of SEQ ID NO.: 4);

SEQ ID NO:61 sets out the amino acid sequence encoded by the wzy gene of DSM 33225 (nucleotides 6955-8145 of SEQ ID NO.: 4);

SEQ ID NO:62 sets out the amino acid sequence of a putative glycerophosphotransferase (glyphos trans) family protein of DSM 33225 (nucleotides 8132-9322 of SEQ ID NO.: 4);

SEQ ID NO:63 sets out the amino acid sequence encoded by the wzx gene of DSM 33225 (nucleotides 9309-10727 of SEQ ID NO.: 4);

SEQ ID NO:64 sets out the amino acid sequence encoded by the epsL gene of DSM 33225 (nucleotides 10825-11724 of SEQ ID NO.: 4);

SEQ ID NO:65 sets out the amino acid sequence of the lytR protein of DSM 33225 (nucleotides 11749-12651 of the complementary strand of SEQ ID NO.: 4);

SEQ ID NO:66 sets out the amino acid sequence encoded by the epsR gene of DSM 33133 (nucleotides 1-318 of SEQ ID NO.: 5);

SEQ ID NO:67 sets out the amino acid sequence encoded by the epsX gene of DSM 33133 (nucleotides 407-826 of SEQ ID NO.: 5);

SEQ ID NO:68 sets out the amino acid sequence encoded by the epsC gene of DSM 33133 (nucleotides 993-1772 of SEQ ID NO.: 5);

SEQ ID NO:69 sets out the amino acid sequence encoded by the epsD gene of DSM 33133 (nucleotides 1782-2477 of SEQ ID NO.: 5);

SEQ ID NO:70 sets out the amino acid sequence encoded by the epsB gene of DSM 33133 (nucleotides 2532-3296 of SEQ ID NO.: 5);

SEQ ID NO:71 sets out the amino acid sequence encoded by the epsE gene of DSM 33133 (nucleotides 3318-3998 of SEQ ID NO.: 5);

SEQ ID NO:72 sets out the amino acid sequence of a putative glycosyltransferase (GT1) of DSM 33133 (nucleotides 4008-4478 of SEQ ID NO.: 5);

SEQ ID NO:73 sets out the amino acid sequence of a putative glycosyltransferase (GT2) of DSM 33133 (nucleotides 4478-4960 of SEQ ID NO.: 5);

SEQ ID NO:74 sets out the amino acid sequence of a putative glycosyltransferase (GT3) of DSM 33133 (nucleotides 5015-5965 of SEQ ID NO.: 5);

SEQ ID NO:75 sets out the amino acid sequence of a putative glycosyltransferase (GT4) of DSM 33133 (nucleotides 6026-6955 of SEQ ID NO.: 5);

SEQ ID NO:76 sets out the amino acid sequence encoded by the wzy gene of DSM 33133 (nucleotides 6955-8145 of SEQ ID NO.: 5);

SEQ ID NO:77 sets out the amino acid sequence of a putative glycerophosphotransferase (glyphos trans) family protein of DSM 33133 (nucleotides 8132-9322 of SEQ ID NO.: 5);

SEQ ID NO:78 sets out the amino acid sequence encoded by the wzx gene of DSM 33133 (nucleotides 9309-10727 of SEQ ID NO.: 5);

SEQ ID NO:79 sets out the amino acid sequence encoded by the epsL gene of DSM 33133 (nucleotides 10825-11724 of SEQ ID NO.: 5);

SEQ ID NO:80 sets out the amino acid sequence of the lytR protein of DSM 33133 (nucleotides 11749-12651 of the complementary strand of SEQ ID NO.: 5).

### DETAILED DESCRIPTION

### Definitions

All definitions of herein relevant terms are in accordance of what would be understood by the skilled person in relation to the herein relevant technical context.

In the context of the present invention in any of its embodiments, the expression “lactic acid bacteria” (“LAB”) designates food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of carbohydrate by these bacteria causes the formation of lactic acid, reducing the pH and leading to the formation of a protein coagulum. These bacteria are thus responsible for the acidification of milk and for the texture of the dairy product. The industrially most useful lactic acid bacteria are found within the order “Lactobacillales” which includes Lactococcus spp., Streptococcus spp., Lactobacillus spp., Leuconostoc spp., Pediococcus spp. and Propionibacterium spp. These are frequently used as food cultures alone or in combination with other lactic acid bacteria.

By “texturing strain” in the present specification and claims is meant a strain which preferably generates fermented mammalian milks having, under the conditions described below and as exemplified in Example 1 herein, a shear stress preferably greater than 40 Pa measured at shear rate 300 s⁻¹. A strain of Lactococcus lactis can be defined as strongly texturing in that it generates fermented milks having, under the same conditions, a shear stress greater than 50 Pa measured at shear rate 300 s⁻¹. 200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹ wherein the inoculation temperature is 30° C.

In addition, by “texturing strain” in the present specification and claims is meant a strain which preferably generates fermented plant-based milks having, under the conditions described below and as exemplified in Example 2 herein, a shear stress of 24 Pa or more, preferably 30 Pa or more, or even more preferably 42 Pa or more than measured at shear rate 300 s⁻¹. A strain of Lactococcus lactis can be defined as strongly texturing in that it generates fermented milks having, under the same conditions, a shear stress of 30 Pa or more measured at shear rate 300 s⁻¹. 1% volume overnight microbial culture (obtained by inoculating the microbial culture in M17 broth supplemented with 2% glucose at 30° C.) is inoculated in soy milk with glucose, such as 0.5-5% glucose, preferably 0.5-2% glucose, more preferably about 2% glucose, as described in Example 2. The inoculation takes place at 30° C. in 200-ml scale until target pH has been reached e.g. pH between 4 and 5, preferably between pH 4.3 to 4.7, more preferably between pH 4.4 to 4.6, and even more preferably pH 4.45, pH 4.50, or pH 4.55 followed by cooling to 4° C. and storage at 4° C. until shear stress is measured, typically from 1-7 days, such as for 5 days, followed by gently stirring and measuring the shear stress at shear rate 300 s⁻¹ wherein the inoculation temperature is 30° C.

The texturing lactic acid bacterium strain of the invention may be an isolated strain, e.g., isolated from a naturally occurring source, or may be a non-naturally occurring strain, e.g., obtained recombinantly. Recombinant strains will differ from naturally occurring strains by at least the presence of the nucleic acid construct(s) used to transform or transfect the mother strain.

The term “sequence identity” relates to the relatedness between two nucleotide sequences or between two amino acid sequences. For purposes of the present invention, the degree of sequence identity between two nucleotide sequences or two amino acid sequences is determined using multiple sequence alignment tool Clustal Omega (https://www.ebi.ac.uk/Tools/msa/clustalo/; Sievers, F. et al., 2011, “Fast, scalable generation of high-quality protein multiple sequence alignments using Clustal Omega”, Mol. Syst. Biol., 7:539) with standard parameters.

In the present context, the terms “strains derived from”, “derived strain” or “mutant” should be understood as a strain derived from a strain of the invention by means of, e.g., genetic engineering, radiation and/or chemical treatment, and/or selection, adaptation, screening, etc. It is preferred that the derived strain is a functionally equivalent mutant, e.g., a strain that has substantially the same, or improved, properties with respect to texturing capacity as the mother strain. Such a derived strain is a part of the present invention. Especially, the term “derived strain” or “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, no more than 10, or no more than 5, treatments are carried out. In a presently preferred derived strain, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

The term “thermophilic” herein refers to microorganisms that thrive best at temperatures above 35° C. The industrially most useful thermophilic bacteria include Streptococcus spp. and Lactobacillus spp. The term “thermophilic fermentation” herein refers to fermentation at a temperature above about 35° C., such as between about 35° C. to about 45° C. The term “thermophilic fermented milk product” refers to fermented milk products prepared by thermophilic fermentation of a thermophilic starter culture and include such fermented milk products as set-yoghurt, stirred-yoghurt and drinking yoghurt, e.g., Yakult. In addition, the term “thermophilic fermented milk product” refers to fermented milk products prepared by thermophil fermentation of a thermophilic starter culture in a plant-based milk base, such as soy milk or soy milk supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

The term “mesophilic” herein refers to microorganisms that thrive best at moderate temperatures (15° C.-35° C.). The industrially most useful mesophilic bacteria include Lactococcus spp. and Leuconostoc spp. The term “mesophilic fermentation” herein refers to fermentation at a temperature between about 22° C. and about 35° C. The term “mesophilic food products” refers to food products prepared by mesophilic fermentation of a mesophilic starter culture. The term “mesophilic fermented milk product” refers to fermented milk products prepared by mesophilic fermentation of a mesophilic starter culture and include such fermented milk products as buttermilk, sour milk, cultured milk, smetana, sour cream, Kefir and fresh cheese, such as quark, tvarog, cream cheese and plantgurt. In addition, the term “mesophilic fermented milk product” refers to fermented milk products prepared by mesophilic fermentation of a mesophilic starter culture in a plant-based milk base, such as soy milk or soy milk supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

The term “mesophilic starter culture” herein refers to any starter cultures culture containing at least one mesophilic bacterium strain. Mesophilic starter cultures, such as combinations of Lactococcus lactis subsp lactis strains and Lactococcus lactis subsp. cremoris strains, are used to produce fermented milk products, such as fresh cheese, butter milk, sour milk and sour cream.

The terms “fermented milk” and “dairy” are used interchangeably herein. In the context of the present invention in any of its embodiments, the expression “fermented milk product” means a food or feed product wherein the preparation of the food or feed product involves fermentation of a milk base with a lactic acid bacterium. “Fermented milk product” as used herein includes but is not limited to products such as thermophilic fermented milk products or mesophilic fermented milk products, as defined above. In addition, as described above “fermented milk product” as used herein includes products prepared by fermentation of plant-based milk bases, such as soy milk or soy milk supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose. Hence, “fermented milk product” according to the present invention encompass fermented mammalian-milk products (i.e., the milk base has mammalian origin) and fermented plant-milk product (i.e., the milk base is a plant-derived milk base, such as soy milk base).

In the context of the present application, the term “milk” is broadly used in its common meaning to refer to liquids produced by the mammary glands of animals (e.g., cows, sheep, goats, buffaloes, camel, etc.) or produced by plants. The term “milk base” or “milk substrate” may be any milk material that can be subjected to fermentation according to the present invention. Thus, useful milk bases include, but are not limited to, solutions/-suspensions of any milk or milk like products comprising protein, such as whole or low-fat milk, skim milk, buttermilk, reconstituted milk powder, condensed milk, dried milk, whey, whey permeate, lactose, mother liquid from crystallization of lactose, whey protein concentrate, cream, or plant-based milks. Obviously, the milk base may originate from any mammalian, e.g., being substantially pure mammalian milk, or reconstituted milk powder. Plant sources of milk include, but are not limited to, milk extracted from soy bean. Preferably, the plant-based milk is soy milk, which can be preferably supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

Prior to fermentation, the milk base may be homogenized and pasteurized according to methods known in the art. “Homogenizing” as used in the context of the present invention in any of its embodiments, means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used in the context of the present invention in any of its embodiments, means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow. For instance, milk base may be heat treated at 92° C. for 3 min, cooled to 38° C. and then inoculated as described in step i. of the process of the present invention.

As used herein, the term “about” (or “around”) means the indicated value ±1% of its value, or the term “about” means the indicated value ±2% of its value, or the term “about” means the indicated value ±5% of its value, the term “about” means the indicated value ±10% of its value, or the term “about” means the indicated value ±20% of its value, or the term “about” means the indicated value ±30% of its value; preferably the term “about” means exactly the indicated value (±0%).

Throughout the description and claims the word “comprise” and variations of the word (e.g., “comprising”, “having”, “including”, “containing”) typically is not limiting and thus does not exclude other features, which may be for example technical features, additives, components, or steps. However, whenever the word “comprise” is used herein, this also includes a special embodiment in which this word is understood as limiting; in this particular embodiment the word “comprise” has the meaning of the term “consist of”.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Texture is an important quality factor for fermented milk products such as yoghurt, and consumer acceptance is often closely linked to texture properties. The texture of fermented milk is dependent on both the bacteria used for fermentation and process parameters. Polysaccharide-producing bacteria can positively influence product characteristics such as texture and sensory properties. Sensory textural attributes are often correlated with the results from instrumental text, e.g., shear stress is related to viscosity and perceived mouth thickness (Poulsen et al., 2019). In the context of the present invention, the rheological properties (texture) of a fermented milk product, such as viscosity, can be measured as a function of shear stress of the fermented milk product, as described below.

In connection with the present invention, shear stress may be measured by the following method: When the pH of the fermented milk (e.g., mammalian- or plant-based milk) reached pH^{˜}4.55, the fermented milk product was brought to 4° C. and manually stirred gently by means of a stick fitted with a perforated disc until homogeneity of the sample. The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:
Holding time (to rebuild to somewhat original structure)
5 minutes without any physical stress (oscillation or rotation) applied to the sample.
Oscillation step (to measure the elastic and viscous modulus, G′ and G″, respectively, therefore calculating the complex modulus G*)

Constant strain=0.3%, frequency (f)=[0.5 . . . 8] Hz
6 measuring points over 60 s (one every 10 s)
Rotation step (to measure shear stress at 300 1/s)
Two steps were designed:
Shear rate=[0.3−300] 1/s and 2) Shear rate=[275−0.3] 1/s.

Each step contained 21 measuring points over 210 s (on every 10 s). The shear stress at 300 1/s (300 s⁻¹) was chosen for further analysis, as this correlates to mouth thickness when swallowing a fermented milk product.

Preferably, the shear stress is measured by the following method: Shear stress data were obtained by inoculating the same microbial cultures in semi-fat milk (1.5% fat); milk was heated at 90° C. for 20 min and cooled down to the inoculation temperature (30° C.), prior to inoculation with 1% overnight microbial culture. The inoculation took place for 8-22 h at 30° C. in 200-ml scale until pH^{˜}4.55 followed by cooling to 4° C. and storage at 4° C. until shear stress was measured, typically from 1-7 days, such as for 5 days. After the storage, the fermented milk was stirred gently by means of a stick fitted with a bored disc until homogeneity of the sample. Shear stress of the samples was assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

Alternatively, the shear stress is measured by the following method: 1% volume overnight microbial culture (obtained by inoculating the microbial culture in M17 broth supplemented with 2% glucose at 30° C.) is inoculated in soy milk with glucose, such as 0.5-5% glucose, preferably 0.5-2% glucose, more preferably about 2% glucose. The inoculation takes place at 30° C. in 200-ml scale until pH^{˜}4.55, followed by cooling to 4° C. and storage at 4° C. until shear stress is measured, typically from 1-7 days, such as for 5 days. After the storage, the fermented milk is stirred gently by means of a stick fitted with a bored disc until homogeneity of the sample. Shear stress is assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ may be chosen.

The Lactococcus lactis Lactic Acid Bacterium (LAB) Strains

It is an object of the present invention to provide texturing LAB strains suitable for use in preparation of food products. In particular, it is an object of the present invention to provide texturing Lactococcus lactis strains suitable for use in preparation of mesophilic food products. This object has been solved with the Lactococcus lactis strains as described herein. As discussed in the examples (see, e.g., Tables 1, 2 and 3, and Examples land 2), the disclosed Lactococcus lactis strains DSM 33204, DSM 33205, DSM 33220, DSM 33221, DSM 33218, DSM 33219, DSM 33224, DSM 33197, DSM 33196, DSM 33195, DSM 33194, DSM 33226, DSM 33193, DSM 33200, DSM 33201, DSM 33202, DSM 33203, DSM 33222, DSM 33225, DSM 33133, DSM 33223 and DSM 33192 have excellent texturing properties.

The present inventors analyzed the eps gene cluster of the above strains and identified gene sequences which are believed to be involved in the production of exopolysaccharide (EPS), and thereby involved in the creation of the excellent texturing properties of the above Lactococcus lactis strains for fermenting milk.

In LAB, the Wzy-dependent pathway is the pathway of choice for the synthesis of heteropolymeric EPS. The genetic loci for polysaccharide biosynthesis by the Wzy-dependent mechanism are similar in all bacteria and are well studied in Streptococcus pneumoniae. Of note, S. pneumoniae produces only capsular exocellular polysaccharides (often abbreviated as CPS), while LAB can produce both CPS and EPS (EPS stands for “exocellular polysaccharide”, which is excreted into the medium/milk). The same gene cluster is responsible for the production of CPS and EPS. Genetic analysis of the CPS locus from 90 pneumococcal serotypes demonstrated a striking feature of the polysaccharide operon: the presence of many highly divergent forms of each of the key enzyme classes. Thus, there were found 40 homology groups for polysaccharide polymerases, 13 groups of lipases, and a great diversity of glycosyltransferases. The presence of multiple non-homologous or highly divergent forms of these enzymes, together with often different G+C content of the region in which these are encoded, supports the view that these genes have been imported on multiple occasions from different and unknown sources. Many eps gene clusters have undergone rearrangement mediated by insertion sequence (IS) elements and received genes from other organisms by a horizontal gene transfer. Typical of eps operon organization is the presence of IS elements flanking or within the operon. The plethora of glycosyltransferases observed in the loci for polysaccharide production provides an opportunity to continually generate new strains producing unique EPS by gene shuffling. As EPS show an enormous diversity in monosaccharide building blocks, anomeric configuration, conformation, and stereochemistry, the resulting diversity of EPS structures is uncanny: for instance, two glucose residues can be joined together in 30 different ways. According to Carbohydrate-Active enZymes (CAZy) database (cazy.org), glycosyltransferases are currently classified into 107 families (June 2019, http://www.cazy.org/GlycosylTransferases.html), which can help in predicting their mode of action. Nevertheless, this does not mean that all enzymes of a family recognize the same donor and acceptor, as polyspecificity is common among glycosyltransferase families, and thus one should be prudent with the over-interpretation of predictions based purely on this classification.

Genes encoding Wzy-dependent exocellular polysaccharide biosynthesis proteins in LAB are typically organized in a cluster with an operon structure and are generally chromosomal in Streptococcus thermophilus, but can reside on a plasmid or the chromosome in L. lactis and Lactobacillus sp. Generally, eps gene clusters are highly diverse, and their nucleotide sequences are among the most variable sequences in LAB genomes. However, the modular gene organization in eps gene clusters is conserved (Zeidan et al., 2017). The conserved genes in the beginning of the eps gene cluster, which are involved in the modulation and assembly machinery of polysaccharide biosynthesis, were denominated epsRXCDB, according to the nomenclature by Zeidan et al. (2017) and Poulsen et al. (2019), and those at the end, epsL and lytR, while the polymerase was named wzy, and the flippase, wzx. The genes of the variable part include polymerase wzy, polysaccharide transporter also called flippase wzx, and glucosyltransferases (GT) or other polymer-modifying enzymes. The common denominator for the texturing strains is that they all contain the genes required for the polysaccharide production, e.g. epsCDBE-wzy-wzx and GT (Zeidan et al., 2017). No putative function could be yet assigned to epsX and epsL. NIZO B40 epsL can be disrupted by single crossover using an internal gene fragment or overproduced without any effect on EPS production (van Kranenburg, 1999). However, it might be that the second copy of epsL takes over, if the one from the eps cluster is not functional.

EpsR is believed to be responsible for EPS biosynthesis regulation, and thus certain mutations would affect the EPS production. EpsCDB and ATP are believed to form a stable complex acting as a tyrosine kinase—phosphatase system, which controls EPS synthesis, likely through the phosphorylation of EpsE, a glycosylphospho-transferase that catalyzes the first step in the assembly of the EPS repeat unit and defines the type of sugar added to the lipid carrier for the formation of EPS. All three genes responsible for tyrosine phosphorylation are essential for the complete encapsulation of the pneumococcus, with cpsC (corresponding to epsC in L. lactis) being a major virulence factor, crucial via its role in the regulation of the CPS biosynthesis (Whittall et al., 2015). In L. lactis, epsC and epsD were found to be essential for the EPS production, while epsB was not strictly required, as the effect of its deletion was the reduced amount of EPS produced (Nierop Groot and Kleerebezem, 2007). Gene epsE encoding the initial glycose phosphate transferase, which does not catalyze glycosidic linkage, but is involved in linking the first sugar of the repeat unit to the lipid carrier, was shown to be essential for polysaccharide biosynthesis in L. lactis, as its disruption abolished EPS production (Dabour and LaPointe 2005, van Kranenburg et al., 1997).

Subsequently, the following genes of the eps cluster typically encoding glycosyltransferases, polymerases and transporters, are situated in a variable part of the cluster and do often have a low degree of similarity to already characterized genes, which makes the prediction of their putative functions difficult. Comparison of polysaccharide synthesis operons from 90 pneumococcal serotypes, where polysaccharide biosynthesis is well studied, revealed that central genes responsible for the synthesis and polymerization of the repeat unit are highly variable and often non-homologous between serotypes (Bentley et al., 2006). Wzy-dependent CPS biosynthesis in S. pneumoniae resembles peptidoglycan synthesis, whereby repeat units are built on the innerface of the cytoplasmic membrane, transported to the outer face of the membrane by a Wzx transporter, also called flippase, and polymerized by a Wzy polymerase. The polysaccharide polymerase Wzy links individual repeat units to form lipid-linked CPS. In S. pneumonia, 40 homology groups for polysaccharide polymerases were found. The initial sugar of the repeat oligosaccharide unit is also the donor sugar in the polymerization of the repeat units, and the specificity of the Wzy polymerase determines the linkage type. The predictions for initial sugars, and subsequent repeat-unit polymerization linkage, correlate well with the polymerase homology groups. In S. pneumonia, there are 32 polymerase homology groups associated with WchA, five with WciI, four with WcjG and one with WcjH. These associations are mostly exclusive, with only five polymerase homology groups associated with two initial transferases, which indicates a high specificity of the initial transferases (Bentley et al., 2006).

Without being limited to theory, it is currently believed that differences in the genes related to EPS biosynthesis, specially of the variable region, in particular wzy, wzx, the GT genes, if present, and other oligosaccharide repeating unit modifying genes, are likely to be responsible for the different EPS structures produced by the different LAB strains. This may have an impact on the differences in the texturing capability of the different LAB strains. In addition, without being limited to theory, it is believed that the genetic background of each specific strain may also contribute to the differences in the texturing capability of the different LAB strains.

As discussed above, a first aspect of the present invention relates to a Lactococcus lactis lactic acid bacterium strain comprising an active eps gene cluster capable of producing exopolysaccharide (EPS), wherein the eps gene cluster is selected from:
(i) a nucleotide sequence as defined in SEQ ID NO.: 1;
(ii) a nucleotide sequence as defined in SEQ ID NO.: 2, or a nucleotide sequence which differs by no more than 1 nucleotide from the nucleotide sequence as defined in SEQ ID NO.: 2;
(iii) a nucleotide sequence as defined in SEQ ID NO.: 3, or a nucleotide sequence which differs by no more than 1 nucleotide from the nucleotide sequence as defined in SEQ ID NO.: 3;
(iv) a nucleotide sequence as defined in SEQ ID NO.: 4, or a nucleotide sequence which differs by no more than 1 nucleotide from the nucleotide sequence as defined in SEQ ID NO.: 4; and
(v) a nucleotide sequence as defined in SEQ ID NO.: 5, or a nucleotide sequence which differs by no more than 5 nucleotides, preferably by no more than 4 nucleotides, more preferably by no more than 3 nucleotides, even more preferably by no more than 2 nucleotides, most preferably by no more than 1 nucleotide from the nucleotide sequence as defined in SEQ ID NO.: 5.

In a preferred embodiment, the eps gene cluster of the Lactococcus lactis lactic acid bacterium strain comprising an active eps gene cluster capable of producing exopolysaccharide (EPS), according to the first aspect of the present invention is selected from:
(i) a nucleotide sequence as defined in SEQ ID NO.: 1;
(ii) a nucleotide sequence as defined in SEQ ID NO.: 2;
(iii) a nucleotide sequence as defined in SEQ ID NO.: 3;
(iv) a nucleotide sequence as defined in SEQ ID NO.: 4; and
(v) a nucleotide sequence as defined in SEQ ID NO.: 5.

In a preferred embodiment, the Lactococcus lactis LAB strain comprising an active eps gene cluster capable of producing exopolysaccharide, wherein the eps gene cluster is defined in SEQ ID NO.: 1, belongs to the MLST (multilocus sequence typing) group ST76, wherein the MLST analysis is performed as described in Example 4 of the present description, i.e., with a 12 gene MLST scheme developed at Chr. Hansen. The scheme is based on the twelve genes dnaK, fusA, groEL, gyrA, gyrB, ileS, lepA, pheS, recA, rpoA, rpoB and rpoCchosen from the core genome of Lactobacillaceae (Salvetti et al., 2018). A total of 22493 bp are used in the scheme, which thereby represents almost 1% of the average Lactococcus genome. MLST typing with Illumina whole genome sequences is performed with the help of the CLC Microbial Genomics Module, which is a plugin to the CLC Genomics Workbench v10. In CLC, MLST is incorporated into Chr. Hansen's custom designed standard genome sequence analysis pipeline. It is performed both on de novo contigs and reference assemblies.

Preferably, the Lactococcus lactis LAB strain comprising an active eps gene cluster capable of producing exopolysaccharide, wherein the eps gene cluster is defined in SEQ ID NO.: 2, or wherein the eps gene cluster is defined by a nucleotide sequence which differs by no more than 1 nucleotide from the nucleotide sequence as defined in SEQ ID NO.: 2, belongs to the MLST (multilocus sequence typing) group ST76.

Preferably, the Lactococcus lactis LAB strain comprising an active eps gene cluster capable of producing exopolysaccharide, wherein the eps gene cluster is defined in SEQ ID NO.: 3, or wherein the eps gene cluster is defined by a nucleotide sequence which differs by no more than 1 nucleotide from the nucleotide sequence as defined in SEQ ID NO.: 3, belongs to the MLST (multilocus sequence typing) group ST76.

Preferably, the Lactococcus lactis LAB strain comprising an active eps gene cluster capable of producing exopolysaccharide, wherein the eps gene cluster is defined in SEQ ID NO.: 4, or wherein the eps gene cluster is defined by a nucleotide sequence which differs by no more than 1 nucleotide from the nucleotide sequence as defined in SEQ ID NO.: 4, belongs to the MLST (multilocus sequence typing) group ST76.

Preferably, the Lactococcus lactis LAB strain comprising an active eps gene cluster capable of producing exopolysaccharide, wherein the eps gene cluster is defined in SEQ ID NO.: 5, or wherein the eps gene cluster is defined by a nucleotide sequence which differs by no more than 1 nucleotide, preferably by no more than 4 nucleotides, more preferably by no more than 3 nucleotides, even more preferably by no more than 2 nucleotides, most preferably by no more than 1 nucleotide, from the nucleotide sequence as defined in SEQ ID NO.: 5, belongs to the MLST (multilocus sequence typing) group ST140.

As discussed above, the MLST analysis is performed as described in Example 4, i.e., with a 12 gene MLST scheme developed at Chr. Hansen. The scheme is based on the twelve genes dnaK, fusA, groEL, gyrA, gyrB, ileS, lepA, pheS, recA, rpoA, rpoB and rpoC chosen from the core genome of Lactobacillaceae (Salvetti et al., 2018). A total of 22493 bp are used in the scheme, which thereby represents almost 1% of the average Lactococcus genome. MLST typing with Illumina whole genome sequences is performed with the help of the CLC Microbial Genomics Module, which is a plugin to the CLC Genomics Workbench v10. In CLC, MLST is incorporated into Chr. Hansen's custom designed standard genome sequence analysis pipeline. It is performed both on de novo contigs and reference assemblies.

In a further preferred embodiment, the Lactococcus lactis LAB strain comprising an active eps gene cluster capable of producing exopolysaccharide, wherein the eps gene cluster is defined in SEQ ID NO.: 1 is able to generate fermented milks with a pH of about 4.55 in about 15 h or less (“time-to-pH 4.55” of 15 h or less), preferably in about 13 h or less (“time-to-pH 4.55” of 13 h or less), more preferably in about 12 h or less (“time-to-pH 4.55” of 12 h or less), even more preferably in about 11 h or less (“time-to-pH 4.55” of 11 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis LAB strain comprising an active eps gene cluster capable of producing exopolysaccharide, wherein the eps gene cluster is defined in SEQ ID NO.: 1 is able to generate fermented milks with a pH of about 4.55 (such as a pH of about 4.49, 4.53 or 4.55) in about 21 h or less (“time-to-pH 4.55” of 21 h or less), preferably in about 16 h or less (“time-to-pH 4.55” of 16 h or less), more preferably in about 11 h or less (“time-to-pH 4.55” of 11 h or less), even more preferably in about 8 h or less (“time-to-pH 4.55” of 8 h or less), measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 (such as a pH of about 4.49, 4.53 or 4.55), as described above, is reached.

In a further preferred embodiment, the Lactococcus lactis LAB strain comprising an active eps gene cluster capable of producing exopolysaccharide, wherein the eps gene cluster is defined in SEQ ID NO.: 2, or wherein the eps gene cluster is defined by a nucleotide sequence which differs by no more than 1 nucleotide from the nucleotide sequence as defined in SEQ ID NO.: 2, is able to generate fermented milks with a pH of about 4.55 in about 15 h or less (“time-to-pH 4.55” of 15 h or less), preferably in about 13 h or less (“time-to-pH 4.55” of 13 h or less), more preferably in about 12 h or less (“time-to-pH 4.55” of 12 h or less), even more preferably in about 9 h or less (“time-to-pH 4.55” of 9 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis LAB strain comprising an active eps gene cluster capable of producing exopolysaccharide, wherein the eps gene cluster is defined in SEQ ID NO.: 2 is able to generate fermented milks with a pH of about 4.55 (such as a pH of about 4.54, 4.55 or 4.66) in about 21 h or less (“time-to-pH 4.55” of 21 h or less), preferably in about 11 h or less (“time-to-pH 4.55” of 11 h or less), more preferably in about 10.5 h or less (“time-to-pH 4.55” of 10.5 h or less), measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 (such as a pH of about 4.54, 4.55 or 4.66), as described above, is reached.

In a further preferred embodiment, the Lactococcus lactis LAB strain comprising an active eps gene cluster capable of producing exopolysaccharide, wherein the eps gene cluster is defined in SEQ ID NO.: 3, or wherein the eps gene cluster is defined by a nucleotide sequence which differs by no more than 1 nucleotide from the nucleotide sequence as defined in SEQ ID NO.: 3, is able to generate fermented milks with a pH of about 4.55 in about 14 h or less (“time-to-pH 4.55” of 14 h or less), preferably in about 12 h or less (“time-to-pH 4.55” of 12 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis LAB strain comprising an active eps gene cluster capable of producing exopolysaccharide, wherein the eps gene cluster is defined in SEQ ID NO.: 3 is able to generate fermented milks with a pH of about 4.55 in about 7.5 h or less (“time-to-pH 4.55” of 7.5 h or less, measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 is reached.

In a further preferred embodiment, the Lactococcus lactis LAB strain comprising an active eps gene cluster capable of producing exopolysaccharide, wherein the eps gene cluster is defined in SEQ ID NO.: 4, or by a nucleotide sequence which differs by no more than 1 nucleotide from the nucleotide sequence as defined in SEQ ID NO.: 4, is able to generate fermented milks with a pH of about 4.55 in about 13 h or less (“time-to-pH 4.55” of 13 h or less), preferably in about 11 h or less (“time-to-pH 4.55” of 11 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis LAB strain comprising an active eps gene cluster capable of producing exopolysaccharide, wherein the eps gene cluster is defined in SEQ ID NO.: 4 is able to generate fermented milks with a pH of about 4.55 in about 10 h or less (“time-to-pH 4.55” of 10 h or less, measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 is reached.

In a further preferred embodiment, the Lactococcus lactis LAB strain comprising an active eps gene cluster capable of producing exopolysaccharide, wherein the eps gene cluster is defined in SEQ ID NO.: 5, or wherein the eps gene cluster is defined by a nucleotide sequence which differs by no more than 1 nucleotide, preferably by no more than 4 nucleotides, more preferably by no more than 3 nucleotides, even more preferably by no more than 2 nucleotides, most preferably by no more than 1 nucleotide, from the nucleotide sequence as defined in SEQ ID NO.: 5, is able to generate fermented milks with a pH of about 4.55 in about 10 h or less (“time-to-pH 4.55” of 10 h or less), preferably in about 8 h or less (“time-to-pH 4.55” of 8 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis LAB strain comprising an active eps gene cluster capable of producing exopolysaccharide, wherein the eps gene cluster is defined in SEQ ID NO.: 5 is able to generate fermented milks with a pH of about 4.55 in about 10.5 h or less (“time-to-pH 4.55” of 10.5 h or less, measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 is reached.

The active eps gene clusters as defined in (i) to (v) above (SEQ ID NO.: 1-5) are found in Lactococcus lactis lactic acid bacteria with excellent texturing properties, as disclosed in the examples.

The term “exopolysaccharide (EPS)” is well known and the skilled person can routinely determine if a lactic acid bacterium of interest produces EPS. As known and understood by the skilled person a lactic acid bacterium of interest, which produces EPS, will comprise an active eps gene cluster.

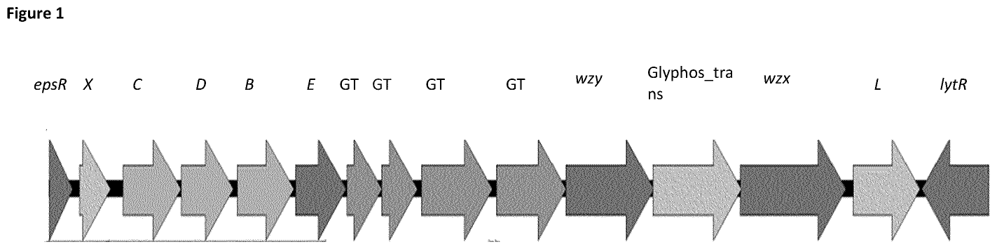

As known to the skilled person, as described above, an active eps gene cluster comprises genes involved in regulation and modulation of EPS biosynthesis and genes involved in the biosynthesis of an oligosaccharide repeat unit and export, including a glycosyltransferase (GT), a polymerase and a transporter. In short and as understood by the skilled person, since the lactic acid bacterium strains of the first aspect are capable of producing and exporting exopolysaccharide (EPS), then they comprise an active eps gene cluster. Zeidan et al. (2017) reviews the production of EPS by LAB, and provide details of the structure of eps gene clusters in LAB. FIG. 1 shows the eps gene cluster in L. lactis cremoris strain B40 (pNZ4000, GenBank AF036485).

Preferably, the Lactococcus lactis lactic acid bacterium (LAB) strain comprising an active eps gene cluster capable of producing exopolysaccharide (EPS), wherein the eps gene cluster has the sequence as defined in SEQ ID NO.: 1 is selected from the following strains:
Strain DSM 33204, or a mutant or variant therefrom;
Strain DSM 33205, or a mutant or variant therefrom;
Strain DSM 33220, or a mutant or variant therefrom;
Strain DSM 33221, or a mutant or variant therefrom;
Strain DSM 33218, or a mutant or variant therefrom;
Strain DSM 33219, or a mutant or variant therefrom;
Strain DSM 33224, or a mutant or variant therefrom;
Strain DSM 33197, or a mutant or variant therefrom;
Strain DSM 33196, or a mutant or variant therefrom;
Strain DSM 33195, or a mutant or variant therefrom;
Strain DSM 33194, or a mutant or variant therefrom;
Strain DSM 33226, or a mutant or variant therefrom;
Strain DSM 33223, or a mutant or variant therefrom;
Strain DSM 33193, or a mutant or variant therefrom; and
Strain DSM 33192, or a mutant or variant therefrom.

The above strains belong to the MLST (multilocus sequence typing) group ST76. The MLST analysis is performed as described in Example 4 below.

Preferably, the Lactococcus lactis lactic acid bacterium (LAB) strain comprising an active eps gene cluster capable of producing exopolysaccharide (EPS), wherein the eps gene cluster has the sequence as defined in SEQ ID NO.: 2 is selected from the following strains:
Strain DSM 33200, or a mutant or variant therefrom;
Strain DSM 33201, or a mutant or variant therefrom;
Strain DSM 33202, or a mutant or variant therefrom; and
Strain DSM 33203, or a mutant or variant therefrom.

Strains DSM 33201 and DSM 33203 belong to the MLST (multilocus sequence typing) group ST76. The MLST analysis is performed as described in Example 4 below.

Preferably, the Lactococcus lactis lactic acid bacterium (LAB) strain comprising an active eps gene cluster capable of producing exopolysaccharide (EPS), wherein the eps gene cluster has the sequence as defined in SEQ ID NO.: 3 is strain DSM 33222, or a mutant or variant therefrom. Strain DSM 33222 belongs to the MLST (multilocus sequence typing) group ST76. The MLST analysis is performed as described in Example 4 below.

Preferably, the Lactococcus lactis lactic acid bacterium (LAB) strain comprising an active eps gene cluster capable of producing exopolysaccharide (EPS), wherein the eps gene cluster has the sequence as defined in SEQ ID NO.: 4 is strain DSM 33225, or a mutant or variant therefrom. Strain DSM 33225 belongs to the MLST (multilocus sequence typing) group ST76. The MLST analysis is performed as described in Example 4 below.

Preferably, the Lactococcus lactis lactic acid bacterium (LAB) strain comprising an active eps gene cluster capable of producing exopolysaccharide (EPS), wherein the eps gene cluster has the sequence as defined in SEQ ID NO.: 5 is strain DSM 33133, or a mutant or variant therefrom. Strain DSM 33133 belongs to the MLST (multilocus sequence typing) group ST140. The MLST analysis is performed as described in Example 4 below.

As discussed in working examples herein (see, e.g., Table 1)—the herein disclosed novel Lactococcus lactis strains have excellent texturing properties in mammalian milk. In addition, as shown in Example 2 Tables 2 and 3, the herein disclosed novel Lactococcus lactis strains have excellent texturing properties in plant-based milk, in particular in soy milk supplemented with glucose, such as 0.5-5% glucose, preferably 0.5-2% glucose, more preferably about 2% glucose.

Preferably, the texturing lactic acid bacterium strains as described herein is a LAB strain which generates fermented milks having a shear stress greater than 50 Pa, more preferably 55 Pa or more, even more preferably greater than 56 Pa, such as about 51 Pa, 55 Pa, 58 Pa, 60 Pa, 61 Pa, 62 Pa, 64 Pa, 65 Pa, 66 Pa, 67 Pa, 69 Pa, 70 Pa, 72 pa, 75 Pa, 80 Pa, 85 Pa, 86 Pa, 87 Pa, 88 Pa, 89 Pa, 90 Pa, 95 Pa, 100 Pa, 105 Pa, 110 Pa, 115 Pa, 120 Pa, 121 Pa or more, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. for 5 days, followed by gently stirring and measuring the shear stress at shear rate 300 s⁻¹, wherein the inoculation temperature is 30° C. The shear stress is measured using the method indicated in Example 1.

In addition, the texturing lactic acid bacterium strains as described herein is a LAB strain which generates fermented milks having a shear stress greater than 24 Pa, such as about 35 Pa, 36 Pa, 45 Pa, 47 Pa, 54 Pa, 56 Pa, 57 Pa, 60 Pa, 62 Pa, 63 Pa, 64 Pa, 71 Pa, 74 Pa, 75 Pa, 79 Pa, 86 Pa, 88 Pa, 93 Pa, 96 Pa, 99 Pa, 102 Pa, 106 Pa or more, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH^{˜}4.55 (such as pH 4.49, 4.53, 4.54, 4.55 or 4.66), followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

Preferably the LAB strain generates fermented milk having a shear stress of 55 Pa or more, preferably more than 56 Pa, such as about 58 Pa, 60 Pa, 64 Pa, 65 Pa, 70 Pa, 75 Pa, 80 Pa, 85 Pa, 88 Pa, 90 Pa, 95 Pa, 98 Pa, preferably in the presence of a co-acidifier or helper strain, which is preferably strain DSM 25485, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

In a preferred embodiment, the present invention provides the following Lactococcus lactis lactic acid bacterium (LAB) strains, which are able to generate fermented milks with a pH of about 4.55 in about 13 h or less (“time-to-pH 4.55” of 13 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached:
Strain DSM 33193, or a mutant or variant therefrom
Strain DSM 33204, or a mutant or variant therefrom;
Strain DSM 33205, or a mutant or variant therefrom;
Strain DSM 33220, or a mutant or variant therefrom;
Strain DSM 33221, or a mutant or variant therefrom;
Strain DSM 33218, or a mutant or variant therefrom;
Strain DSM 33219, or a mutant or variant therefrom;
Strain DSM 33224, or a mutant or variant therefrom;
Strain DSM 33197, or a mutant or variant therefrom;
Strain DSM 33196, or a mutant or variant therefrom;
Strain DSM 33200, or a mutant or variant therefrom;
Strain DSM 33201, or a mutant or variant therefrom;
Strain DSM 33203, or a mutant or variant therefrom;
Strain DSM 33222, or a mutant or variant therefrom;
Strain DSM 33225, or a mutant or variant therefrom; and
Strain DSM 33133, or a mutant or variant therefrom.

Since the above strains are able to acidify milk (i.e., reach a target pH—e.g. pH 4.55 as described above) in about 13 h or less, measured as described above, they may be referred to as “fast-acidifying” strains. Target pH may be e.g. pH between 4 and 5, preferably between pH 4.3 to 4.7, more preferably between pH 4.4 to 4.6, and even more preferably pH 4.45, pH 4.50, or pH 4.55. These strains may thus be used on their own or in combination with other strains for the generation of fermented milks and fermented milk products.

In a further preferred embodiment, the present invention provides the following Lactococcus lactis lactic acid bacterium (LAB) strains, which are not able to generate fermented milks with a pH of about 4.55 in about 13 h or less (“time-to-pH 4.55” of more than 13 h), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached:
Strain DSM 33226, or a mutant or variant therefrom;
Strain DSM 33194, or a mutant or variant therefrom;
Strain DSM 33195, or a mutant or variant therefrom;
Strain DSM 33192, or a mutant or variant therefrom;
Strain DSM 33223, or a mutant or variant therefrom; and
Strain DSM 33202, or a mutant or variant therefrom.

Since the above strains are not able to acidify milk (i.e., reach a target pH—e.g. pH 4.55 as described above) in about 13 h or less, measured as described above, they may be referred to as “slow-acidifying” strains. Target pH may be e.g. pH between 4 and 5, preferably between pH 4.3 to 4.7, more preferably between pH 4.4 to 4.6, and even more preferably pH 4.45, pH 4.50, or pH 4.55. Without being limited to theory, for the production of fermented milks, it is currently preferred that milk fermentation (acidification) occurs as fast as possible, e.g., in order to avoid the growth of any potential contaminant microorganism. Accordingly, it is preferred that the above strains are used in combination with a further lactic acid bacterium strain, which, in the context of the present invention, is referred to as “co-acidifier” or “helper” strain. The co-acidifier or helper strain would help the “slow-acidifying” strains to acidify milk in lower amount of time.

Without being limited to theory, it is currently believed that the co-acidifier or helper strain would inter alia metabolize the proteins present in milk (casein) faster than the “slow-acidifying” strain, so that the “slow-acidifying” strain would have more available nitrogen source for their growth, which would then be facilitated. LAB require an exogenous source of amino acids or peptides, which are provided by the proteolysis of milk proteins, e.g., casein, which the most abundant protein in milk and the main source of amino acids (Savijoki, K., et al., Appl Microbiol Biotechnol (2006) 71: 394-406).

Slow-acidifying strains are often associated with low proteolytic activity. Proteolysis is the breakdown of proteins into smaller polypeptides or amino acids. A cell wall proteinase (Prt) hydrolyses milk proteins, such as casein, providing a nitrogen source, which makes milk suitable for rapid growth of strains. Other factors than the prt activity, such as carbon metabolism, Idh and codY activities can also play a role. It is not enough to have high prt activity to acidify milk fast. Uptake and further degradation of peptides are also important for the milk acidification rate. Moreover, EPS production is a highly energy demanding process (Zeidan et al., 2017). Texturing L. lactis strains are generally slower in acidifying milk than the non-texturing strains (Poulsen et al., 2019).

The “co-acidifier” or “helper” strain according with the present invention may be any lactic acid bacterium strain which is able to:
i) generate fermented milks with a pH of about 4.55 in 15 h or less, preferably in 12 h or less (“time-to-pH 4.55” of 15 h or less, preferably 12 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a pH of about 4.55 is reached. Therefore, the “time-to-pH 4.55” can be calculated for a certain lactic acid bacterium strain; and
ii) generate fermented milks having a shear stress of 40 Pa or more measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. until shear stress is measured, typically from 1-7 days, such as for 5 days, followed by gently stirring and measuring the shear stress at shear rate 300 s⁻¹, wherein the inoculation temperature is 30° C.

As shown in the examples, e.g., Table 1, the combination of strains DSM 33226, DSM 33194 or DSM 33195 with a co-acidifier or helper strain, e.g., strain DSM 25485, leads to faster acidification times and/or fermented milks having higher viscosity (measured as shear stress at shear rate 300 s⁻¹, as described above).

In a preferred embodiment, the co-acidifier or helper strain is a lactic acid bacterium strain Lactococcus lactis comprising an active eps gene cluster capable of producing exopolysaccharide (EPS), wherein the eps gene cluster comprises the nucleotide sequences (a), (b) and (c) ((a) to (c4)) as defined in (vi):
(vi) (a): a nucleotide sequence encoding a polypeptide having polymerase activity and having at least 95% identity with the amino acid sequence encoded by nucleotides 6955-8145 of SEQ ID NO: 1 (herein termed wzy);
   (b): a nucleotide sequence encoding a polypeptide having polysaccharide transporter activity and having at least 95% identity with the amino acid sequence encoded by nucleotides 9309-10727 of SEQ ID NO: 1 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 4008-4478 of SEQ ID NO: 1 (herein termed GT1);
   (c2): a nucleotide sequence having at least 95% identity with the amino acid sequence encoded by nucleotides 4478-4960 of SEQ ID NO: 1 (herein termed GT2);
   (c3): a nucleotide sequence having at least 95% identity with the amino acid sequence encoded by nucleotides 5015-5965 of SEQ ID NO: 1 (herein termed GT3); and
   (c4): a nucleotide sequence having at least 95% identity with the amino acid sequence encoded by nucleotides 6026-6955 of SEQ ID NO: 1 (herein termed GT4).

In a further preferred embodiment, a lactic acid bacterium strain Lactococcus lactis comprising an active eps gene cluster capable of producing exopolysaccharide (EPS), wherein the eps gene cluster is as defined in (vii):
(vii) a nucleotide sequence as defined in SEQ ID NO.: 5.

The skilled person would be able to find further co-acidifier or helper strains suitable for the present invention. For instance, a suitable co-acidifier or helper strain may be a lactic acid bacterium strain Lactococcus lactis comprising an active eps gene cluster capable of producing exopolysaccharide (EPS), wherein the eps gene cluster is as defined in SEQ ID NO.: 1-4, wherein the co-acidifier or helper strain is able to (i) generate fermented milks with a pH of about 4.55 in about 15 h or less, preferably in about 12 h or less and is able to (ii) generate fermented milks having a shear stress of 40 Pa or more measured at shear rate 300 s⁻¹, measured as described above.

For instance, the following strains may be used as co-acidifier or helper strains in the context of the present invention: strains DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142 and/or DSM 33137, preferably strains DSM 33193, DSM 33196, DSM 33197, DSM 33200, DSM 33201, DSM 33205, DSM 33218, DSM 33220, DSM 33221, DSM 33222, DSM 33224, DSM 33225, and/or DSM 33137.

More preferably, the co-acidifier or helper strain is a lactic acid bacterium strain is selected from:
(i) the lactic acid bacterium strain Lactococcus lactis subsp. cremoris DSM 25485, or a mutant or variant therefrom; and/or
(ii) the lactic acid bacterium strain Lactococcus lactis subsp. lactis DSM 33192, or a mutant or variant therefrom; and/or
(iii) the lactic acid bacterium strain Lactococcus lactis DSM 33133, or a mutant or variant therefrom.

Accordingly, the present invention further provides the use of any one of strains DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142 and/or DSM 33137, preferably the use of any one of strains DSM 33193, DSM 33196, DSM 33197, DSM 33200, DSM 33201, DSM 33205, DSM 33218, DSM 33220, DSM 33221, DSM 33222, DSM 33224, DSM 33225, and/or DSM 33137 as a co-acidifier or helper strain.

### The Composition Comprising a LAB of the Invention

In a second aspect, the present invention provides a composition comprising one or more of the Lactococcus lactis strains of the invention as described in the first aspect of the present invention.

In particular, the present invention provides a composition comprising one or more of the texturing Lactococcus lactis strains of the invention as described in the first aspect of the present invention and a co-acidifier or helper strain as defined in the first aspect of the present invention. In a preferred embodiment, the composition of the present invention comprises one or more of the Lactococcus lactis strains of the invention as described in the first aspect of the present invention and a co-acidifier or helper strain as defined in the first aspect of the present invention in a ratio of about 9:1 (LAB strain(s) of the present invention: co-acidifier or helper strain(s)).

Preferably, the composition of the present invention comprises at least one Lactococcus lactis lactic acid bacterium strain according to the first aspect of the present invention and one or more further lactic acid bacterium strain(s), wherein the one or more further lactic acid bacterium strain(s) is(are) able to:
i) generate fermented milks with a pH of about 4.55 in about 15 h or less, preferably in about 12 h or less, measured under the following conditions:
   200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a pH of about 4.55 is reached. Therefore, the “time-to-pH 4.55” can be calculated for a certain lactic acid bacterium strain; and
ii) generate fermented milks having a shear stress of 40 Pa or more measured at shear rate 300 s⁻¹, measured under following conditions:
   200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. for 5 days, followed by gently stirring and measuring the shear stress at shear rate 300 s⁻¹, wherein the inoculation temperature is 30° C., wherein the shear stress is measured as described in the present description, e.g., Example 1.

More preferably, the composition of the present invention comprises at least one Lactococcus lactis lactic acid bacterium strain according to the first aspect of the present invention and (a) a lactic acid bacterium strain Lactococcus lactis comprising an active eps gene cluster capable of producing exopolysaccharide (EPS), wherein the eps gene cluster comprises the nucleotide sequences (a), (b) and (c) (a to c4) as defined in (vi), or (b) a lactic acid bacterium strain Lactococcus lactis comprising an active eps gene cluster capable of producing exopolysaccharide (EPS), wherein the eps gene cluster is as defined in (vii):
(vi) (a): a nucleotide sequence encoding a polypeptide having polymerase activity and having at least 95% identity with the amino acid sequence encoded by nucleotides 6955-8145 of SEQ ID NO: 1 (herein termed wzy);
   (b): a nucleotide sequence encoding a polypeptide having polysaccharide transporter activity and having at least 95% identity with the amino acid sequence encoded by nucleotides 9309-10727 of SEQ ID NO: 1 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 4008-4478 of SEQ ID NO: 1 (herein termed GT1);
   (c2): a nucleotide sequence having at least 95% identity with the amino acid sequence encoded by nucleotides 4478-4960 of SEQ ID NO: 1 (herein termed GT2);
   (c3): a nucleotide sequence having at least 95% identity with the amino acid sequence encoded by nucleotides 5015-5965 of SEQ ID NO: 1 (herein termed GT3); and
   (c4): a nucleotide sequence having at least 95% identity with the amino acid sequence encoded by nucleotides 6026-6955 of SEQ ID NO: 1 (herein termed GT4);
(vii) a nucleotide sequence as defined in SEQ ID NO.: 5.

In a further preferred embodiment, the composition of the present invention comprises at least one Lactococcus lactis lactic acid bacterium strain according to the first aspect of the present invention and one or more lactic acid bacterium strains selected from strains DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142, DSM 33137, DSM 33192 and/or DSM 25485, preferably selected from strains DSM 33193, DSM 33196, DSM 33197, DSM 33200, DSM 33201, DSM 33205, DSM 33218, DSM 33220, DSM 33221, DSM 33222, DSM 33224, DSM 33225, DSM 33137, DSM 33192 and/or DSM 25485.

More preferably, the composition of the present invention comprises at least one Lactococcus lactis lactic acid bacterium strain according to the first aspect of the present invention and
(i) the lactic acid bacterium strain Lactococcus lactis subsp. cremoris DSM 25485, or a mutant or variant therefrom; and/or
(ii) the lactic acid bacterium strain Lactococcus lactis subsp. lactis DSM 33192, or a mutant or variant therefrom; and/or
(iii) the lactic acid bacterium strain Lactococcus DSM 33133, or a mutant or variant therefrom.

For example, the composition of the present invention comprises strain DSM 33195 and strain DSM 25485. For example, the composition of the present invention comprises strain DSM 33226 and strain DSM 25485. For example, the composition of the present invention comprises strain DSM 33194 and strain DSM 25485. For example, the composition of the present invention may comprise one or more of strains DSM 33202, DSM 33203, DSM 33204, DSM 33219 and/or strain DSM 33223 and one or more of the co-acidifier or helper strains as defined in the first aspect of the present invention, preferably one or more of the following strains: DSM 25485, DSM 33192 and/or DSM 33133.

In a further embodiment, the composition of the present invention may comprise strain DSM 33226 and strain DSM 24649. In a further embodiment, the composition of the present invention may comprise strain DSM 33194 and strain DSM 24649.

Preferably, the composition of the present invention in any of its embodiments comprises at least 1×10⁶ CFU (colony-forming units)/ml total LAB strains. It may be preferred that the composition comprises at least 1×10⁸ CFU/ml total LAB strains.

As described above in the context of the first aspect of the present invention, the LAB of the present invention, either alone or in combination with a co-acidifier or helper strain, preferably LAB strain Lactococcus lactis subsp. cremoris DSM 25485, are able to generate fermented milks having high shear stress and/or to acidify milk (i.e., reach a pH of about 4.55) in less time. Accordingly, the composition of the present invention is able to generate at least the same shear stress as the one described for the LAB of the present invention, either alone or in the presence of a co-acidifier or helper strain, as described in the context of the first aspect of the present invention.

Lactic acid bacteria, including bacteria of the species Lactococcus sp., are normally supplied to the dairy industry either as frozen (F-DVS) or freeze-dried (FD-DVS) cultures for bulk starter propagation or as so-called “Direct Vat Set” (DVS) cultures, intended for direct inoculation into a fermentation vessel or vat for the production of a dairy product, such as a fermented milk product. Such lactic acid bacterial cultures are in general referred to as “starter cultures” or “starters”. Accordingly, the composition of the present invention may be frozen or freeze-dried. In addition, the composition of the present invention may be provided in liquid form. Thus, in one embodiment, the composition is in frozen, dried, freeze-dried or liquid form.

The composition of the present invention may additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the composition according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity, e.g., cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i. e. the production of, e. g., lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the composition of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

In one embodiment wherein the microorganism produces a polysaccharide (such as EPS) which causes a high/ropy texture in the acidified milk product the acidified milk product is produced substantially free, or completely free of any addition of thickener and/or stabilizer, such as pectin (e.g. HM pectin, LM pectin), gelatin, CMC, Soya Bean Fiber/Soya Bean Polymer, starch, modified starch, carrageenan, alginate, and guar gum. By substantially free should be understood that the product comprises from 0% to 20% (w/w) (e.g. from 0% to 10%, from 0% to 5% or from 0% to 2% or from 0% to 1%) thickener and/or stabilizer.

### Use of the LAB Strains for Increasing the Viscosity of a Fermented Milk Product

In a third aspect, the present invention provides the use of the LAB or the present invention, as described in the first aspect, and/or the use of the composition of the present invention, as described in the second aspect, for increasing the viscosity of a fermented milk product. Hence, in the third aspect, the present invention provides a method for increasing the viscosity (i.e., for improving the texture) of a fermented milk product, wherein the method comprises the use of the LAB or the present invention, as described in the first aspect, and/or the use of the composition of the present invention, as described in the second aspect.

As described above, the LAB strains of the present invention as described in the first aspect and the compositions of the present invention, as described in the second aspect, are able to generate fermented milks having a shear stress greater than 50 Pa, preferably greater than 55 Pa, such as greater than 56 Pa, such as about 51 Pa, 55 Pa, 58 Pa, 60 Pa, 61 Pa, 62 Pa, 64 Pa, 65 Pa, 66 Pa, 67 Pa, 69 Pa, 70 Pa, 72 pa, 75 Pa, 80 Pa, 85 Pa, 86 Pa, 87 Pa, 88 Pa, 89 Pa, 100 Pa, 110 Pa, 115 Pa, 120 Pa, 121 Pa or more, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

As described above, the LAB strains of the present invention as described in the first aspect and the compositions of the present invention, as described in the second aspect, are able to generate fermented milks having a shear stress greater than 24 Pa, such as about 35 Pa, 36 Pa, 45 Pa, 47 Pa, 54 Pa, 56 Pa, 57 Pa, 60 Pa, 62 Pa, 63 Pa, 64 Pa, 71 Pa, 74 Pa, 75 Pa, 79 Pa, 86 Pa, 88 Pa, 93 Pa, 96 Pa, 99 Pa, 102 Pa, 106 Pa or more, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH^{˜}4.55 (or higher/lower, if a strain stops acidifying at a higher/lower pH, see Table 3, such as pH 4.49, 4.53, 4.54, 4.55 or 4.66), followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

The LAB strains of the present invention as described in the first aspect and the compositions of the present invention, as described in the second aspect, are able to generate fermented milk having a shear stress of 55 Pa or more, preferably more than 56 Pa, such as about 58 Pa, 60 Pa, 64 Pa, 65 Pa, 70 Pa, 75 Pa, 80 Pa, 90 Pa, 95 Pa, or 98 Pa, preferably in the presence of an acidifying strain which is preferably selected from DSM 25485, DSM 33192 and/or DSM 33133, even more preferably DSM 25485, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For the specific shear stress of milk fermented with the specific LAB strains of the invention, either alone or in the presence of a co-acidifier or helper strain, we refer to the first aspect of the present invention and to Tables 1-3 in the Examples.

As discussed in the context of the first aspect of the present invention, some of the LAB strains of the present invention are able to acidify milk in about 13 h or less (“fast-acidifying” strains), measured as described above, i.e., 200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached. These strains may thus be preferably used on their own or in combination with other strains for the generation of fermented milks, in particular for their use of fermented milk with increased viscosity.

In addition, there are some LAB strains of the present invention that are not able to acidify milk in about 13 h or less, measured as described above, i.e., 200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached. They may be referred to as “slow-acidifying” strains (e.g., DSM 33192, DSM 33226, DSM 33194, DSM 33202, DSM 33223 and/or DSM 33195). These strains may advantageously be used in the presence of a co-acidifier or helper strain as defined in the first aspect of the present invention. In particular, these strains may be advantageously used in the presence of strain DSM 25485, and/or strain DSM 33192, and/or strain DSM 33133, preferably in the presence of strain DSM 25485. As described above, preferably, the one or more of the Lactococcus lactis strains of the invention as described in the first aspect of the present invention and a co-acidifier or helper strain as defined in the first aspect of the present invention are used in combination in a ratio of about 9:1 (LAB strain(s) of the present invention:co-acidifier or helper strain(s)).

As shown in Table 1, when milk is fermented with strains DSM 33226, DSM 33194 and/or DSM 33195, and the co-acidifier strain DSM 25485, the shear stress values of milk are increased and/or the “time-to-pH 4.55” (measured as described above) is decreased. Without being limited to theory, it is believed that, as described above, the proteolytic nature of DSM 25485 allows and/or facilitates the growth of the LAB of the present invention. In addition, it is believed that a combination of the EPS produced by DSM 25485 and the EPS produced by the strain of the present invention may result in the enhanced viscosity of the fermented milk observed, measured as shear stress, as described above.

Without being limited to theory, it is believed that effect in increased in shear stress of milk fermented with one of the LAB of the present invention and the co-acidifier strain DSM 25485 (see Table 1) would also be obtained when milk is incubated with one of the LAB of the present invention and strain DSM 33192. Strain DSM 33192 is also a helper strain and produces EPS with similar structure as the structure of the EPS produced by strain DSM 25485.

In addition, without being limited to theory, it is it is believed that effect in increase in shear stress of milk fermented with one of the LAB of the present invention and the co-acidifier strain DSM 25485 (see Table 1) would also be obtained when milk is incubated with one of the LAB of the present invention, as described above, and one or more of the following strains: DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142, DSM 33137, DSM 33192 and/or DSM 25485, preferably one of the following strains DSM 33193, DSM 33196, DSM 33197, DSM 33200, DSM 33201, DSM 33205, DSM 33218, DSM 33220, DSM 33221, DSM 33222, DSM 33224, DSM 33225, DSM 33137, DSM 33192 and/or DSM 25485. These strains are able to:

i) generate fermented milks with a pH of about 4.55 in about 15 h or less, preferably in about 12 h or less, measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a pH of about 4.55 is reached; and

ii) generate fermented milks having a shear stress of 40 Pa or more measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. until shear stress is measured, typically from 1-7 days, such as for 5 days, followed by gently stirring and measuring the shear stress at shear rate 300 s⁻¹, wherein the inoculation temperature is 30° C.

In a specific embodiment of the third aspect, the present invention provides the use of the Lactococcus lactis subsp. cremoris strain DSM 25485, for increasing viscosity of a fermented milk product.

The present inventors have surprisingly found that strain DSM 25485 generated fermented milks having a shear stress greater than 45 Pa, preferably greater than 50 Pa, more preferably greater than 55 Pa, such as 56 Pa, see Table 1, measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

In this embodiment, advantageously, strain DSM 25485 can be used either alone or in combination with one or more of the LAB of the present invention, described in the first aspect of the present invention. Preferably, strain DSM 25485 is used in combination with one or more of the LAB of the present invention, described in the first aspect of the present invention in a ratio of about 9:1 (LAB strain(s) of the present invention: strain DSM 25485).

In addition, the present inventors have surprisingly found that strain DSM 25485 generated fermented milks having a shear stress greater than 24 Pa, preferably greater than 30 Pa, more preferably greater than 50 Pa, such as 54 Pa, see Table 2, measured at shear rate 300 s⁻¹, measured under following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

In a further specific embodiment of the third aspect, the present invention provides the use of the Lactococcus lactis subsp. lactis strain DSM 33192, for increasing viscosity of a fermented milk product.

The present inventors have surprisingly found that strain DSM 33192 generated fermented milks having a shear stress greater than 40 Pa, preferably greater than 50 Pa, more preferably greater than 80 Pa, even more preferably greater than 90 Pa, such as 94 Pa, see Table 1, measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

In this embodiment, advantageously, strain DSM 33192 can be used either alone or in combination with one or more of the LAB of the present invention, described in the first aspect of the present invention. Preferably, strain DSM 33192 is used in combination with one or more of the LAB of the present invention, described in the first aspect of the present invention in a ratio of about 9:1 (LAB strain(s) of the present invention:strain DSM 33192).

In addition, the present inventors have surprisingly found that strain DSM 33192 generated fermented milks having a shear stress greater than 24 Pa, preferably greater than 30 Pa, more preferably greater than 40 Pa, even more preferably greater than 45 Pa, such as 47 Pa, see Table 2, measured at shear rate 300 s⁻¹, measured under following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

In a specific embodiment of the third aspect, the present invention provides the use of the Lactococcus lactis subsp. cremoris strain DSM 25485 and/or the use of Lactococcus lactis subsp. lactis strain DSM 33192, as co-acidifier or helper strains, preferably for their use in combination with other texturing LAB strains, as defined in the first aspect of the present invention, for increasing viscosity of a fermented milk product. Preferably, strains DSM 33192 and/or DSM 25485 are used in combination with one or more of the LAB of the present invention, described in the first aspect of the present invention in a ratio of about 9:1 (LAB strain(s) of the present invention:strains DSM 33192 and/or DSM 25485).

### Method of Producing a Food Product and Food Product

In a fourth aspect, the present invention relates to a method of producing a food product comprising at least one stage in which at least one lactic acid bacterium strain as defined in the first aspect of the present invention and/or the composition as defined in the second aspect of the present invention is used. The production of the food product is carried out by methods known to the person skilled in the art.

In another embodiment, the present invention relates to a method of producing a food product comprising at least one stage in which the lactic acid bacterium strain Lactococcus lactis subsp. cremoris DSM 25485, or a mutant or variant therefrom is used.

In another embodiment, the present invention relates to a method of producing a food product comprising at least one stage in which the lactic acid bacterium strain Lactococcus lactis subsp. lactis DSM 33192, or a mutant or variant therefrom is used.

“Fermentation” in the context of the present invention in any of its embodiments means the conversion of carbohydrates into alcohols or acids through the action of microorganisms (LAB). Fermentation processes to be used in production of food products such as dairy products are well known and the person of skill in the art will know how to select suitable process conditions, such as temperature, oxygen, amount of microorganism(s) and process time. Obviously, fermentation conditions are selected so as to support the achievement of the present invention, e.g., to obtain a food product, preferably a food product which has an improved texture as compared to a food product produced with a method which does not involve the use of at least one of the LAB as described in the first aspect of the present invention or the use of the composition as described in the second aspect of the present invention, in any of its embodiments.

In one preferred embodiment, the method of the present invention in any of its embodiments comprises fermenting a milk substrate, which can be a mammalian-based milk substrate or a plant-based milk substrate, such as soy milk, with a composition comprising at least 1×10⁶ CFU, preferably at least 1×10⁸ CFU/ml of total LAB strains.

For instance, the method of the present invention comprises fermenting a milk substrate with a composition comprising at least 1×10⁶ CFU, preferably at least 1×10⁸ CFU/ml of one or more of a strain selected from: DSM 33193, DSM 33133, DSM 33196, DSM 33197, DSM 33200, DSM 33201, DSM 33202, DSM 33195, DSM 33203, DSM 33204, DSM 33205, DSM 33218, DSM 33219, DSM 33220, DSM 33221, DSM 33222, DSM 33223, DSM 33224 and/or DSM 33225.

For instance, the method of the present invention comprises fermenting a milk substrate with a composition comprising at least 1×10⁶ CFU, preferably at least 1×10⁸ CFU/ml of strains DSM 33226 and DSM 25485.

For instance, the method of the present invention comprises fermenting a milk substrate with a composition comprising at least 1×10⁶ CFU, preferably at least 1×10⁸ CFU/ml of strains DSM 33194 and DSM 25485.

For instance, the method of the present invention comprises fermenting a milk substrate with a composition comprising at least 1×10⁶ CFU, preferably at least 1×10⁸ CFU/ml of strains DSM 33195 and DSM 25485.

In another preferred embodiment, the method comprises fermenting a milk substrate with the composition as described in the second aspect of the present invention, in any of its embodiments.

Preferably, the food product is a dairy product and the method in any of its embodiments comprises fermenting a milk substrate (also referred to as “milk base” in the context of the present invention) with the at least one LAB strain and/or with the composition according to the invention (first and second aspects, respectively) and/or with strain DSM 25485 and/or with strain DSM 33192.

Preferably, the food product is a dairy product and the method in any of its embodiments comprises fermenting a plant-based milk substrate (also referred to as “plant-based milk base” in the context of the present invention), such as soy milk, preferably soy milk supplemented with glucose, e.g., 0.5-5% glucose, preferably 0.5-2% glucose, more preferably about 2% glucose, with the at least one LAB strain and/or with the composition according to the invention (first and second aspects, respectively).

The food product according to the present invention may advantageously further comprise a thickener and/or a stabilizer, such as pectin (e.g. HM pectin, LM pectin), gelatin, CMC, Soya Bean Fiber/Soya Bean Polymer, starch, modified starch, carrageenan, alginate, and guar gum.

In a specific embodiment the food product is a dairy product, a meat product, a vegetable product, a fruit product or a cereal product. In a preferred embodiment, the food product is a dairy product. In another preferred embodiment, the food product is a plant-based food product, such as fermented soy milk.

The term “dairy product” as used herein refers to a food product produced from milk. As described above, in the context of the present application, the term “milk” is broadly used in its common meaning to refer to liquids produced by the mammary glands of animals (e.g., cows, sheep, goats, buffaloes, camel, etc.) or by plants. In a preferred embodiment, the milk is cow's milk. In accordance with the present invention the milk may have been processed and the term “milk” includes whole milk, skim milk, fat-free milk, low fat milk, full fat milk, lactose-reduced milk, or concentrated milk. Fat-free milk is non-fat or skim milk product. Low-fat milk is typically defined as milk that contains from about 1% to about 2% fat. Full fat milk often contains 2% fat or more. The term “milk” is intended to encompass milks from different mammals and plant sources. Mammalian sources of milk include, but are not limited to cow, sheep, goat, buffalo, camel, llama, mare and deer. Plant sources of milk include, but are not limited to, milk extracted from soy bean. In a specific embodiment, the milk is cow's milk. In another specific embodiment, the milk is a plant-based milk, preferably soy milk, which can be preferably supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

Preferred dairy products according to the invention are fermented milk products and cheese. In a specific embodiment the dairy product is a mesophilic dairy product.

In a particular embodiment of the invention, the fermented milk product is selected from the group consisting of buttermilk, sour milk, cultured milk, Smetana, sour cream, thick cream, cultured cream, ymer, fermented whey, Kefir, Yakult and fresh cheese, such as Quark, tvorog and cream cheese. In particular, the fermented milk product is selected from the group consisting of Quark, sour cream and Kefir. In a preferred embodiment of the invention, the fermented milk product contains a further food product selected from the group consisting of fruit beverage, cereal products, fermented cereal products, chemically acidified cereal products, soy milk products, fermented soy milk products and any mixture thereof. In another preferred embodiment, the fermented milk product is a plant-based fermented milk product, preferably fermented soy milk, e.g. “plantgurt” from “Alpro”.

The fermented milk product typically contains protein in a level of between 1.0% by weight to 12.0% by weight, preferably between 2.0% by weight to 10.0% by weight. In a particular embodiment, sour cream contains protein in a level of between 1.0% by weight to 5.0% by weight, preferably between 2.0% by weight to 4.0% by weight. In a particular embodiment, Quark contains protein in a level of between 4.0% by weight to 12.0% by weight, preferably between 5.0% by weight to 10.0% by weight.

Preferably, the food product has an improved texture (improved viscosity, measured as shear stress at 300 s⁻¹, as described in the present application and, e.g., in Examples 1 and 2) as compared to a food product produced with a comparable method which does not involve the use of at least one of the LAB as described in the first aspect of the present invention and/or the use of the composition as described in the second aspect of the present invention, in any of its embodiments and/or the use of a co-acidifier or helper strain, as defined above, preferably strain DSM 25485 and/or strain DSM 33192.

The invention also relates to a food product, preferably a dairy product, comprising at least one LAB strain as described in the first aspect of the present invention and/or the composition as described in the second aspect of the present invention.

Method for Manufacturing a Lactococcus lactis Lactic Acid Bacterium (LAB) Strain

In a fifth aspect, the present invention provides a method for manufacturing a Lactococcus lactis lactic acid bacterium (LAB) strain which comprises the following steps:
(a) providing a Lactococcus lactis lactic acid bacterium (LAB) strain comprising an active eps gene cluster capable of producing exopolysaccharide (EPS), wherein the eps gene cluster is selected from:
   (i) a nucleotide sequence as defined in SEQ ID NO.: 1;
   (ii) a nucleotide sequence as defined in SEQ ID NO.: 2, or a nucleotide sequence which differs by no more than 1 nucleotide from the nucleotide sequence as defined in SEQ ID NO.: 2;
   (iii) a nucleotide sequence as defined in SEQ ID NO.: 3, or a nucleotide sequence which differs by no more than 1 nucleotide from the nucleotide sequence as defined in SEQ ID NO.: 3;
   (iv) a nucleotide sequence as defined in SEQ ID NO.: 4, or a nucleotide sequence which differs by no more than 1 nucleotide from the nucleotide sequence as defined in SEQ ID NO.: 4; and
   (v) a nucleotide sequence as defined in SEQ ID NO.: 5, or a nucleotide sequence which differs by no more than 5 nucleotides, preferably by no more than 4 nucleotides, more preferably by no more than 3 nucleotides, even more preferably by no more than 2 nucleotides, most preferably by no more than 1 nucleotide from the nucleotide sequence as defined in SEQ ID NO.: 5,
(b) screening for a Lactococcus lactis lactic acid bacterium (LAB) strain which is:
   (i) able to generate fermented milks having a shear stress of 40 Pa or more, preferably 50 Pa or more, such as more than 56 Pa, more preferably 60 Pa or more, even more preferably 65 Pa or more, such as 70 Pa or more, 75 Pa or more, 80 Pa or more, 85 Pa or more, 90 Pa or more, 95 Pa or more, 100 Pa or more or 120 Pa or more, measured at shear rate 300 s⁻¹, measured under following conditions:
   200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C.; and/or
   (ii) able to generate fermented milks having a shear stress greater than 24 Pa, such as about 35 Pa, 36 Pa, 45 Pa, 47 Pa, 54 Pa, 56 Pa, 57 Pa, 60 Pa, 62 Pa, 63 Pa, 64 Pa, 71 Pa, 74 Pa, 75 Pa, 79 Pa, 86 Pa, 88 Pa, 93 Pa, 96 Pa, 99 Pa, 102 Pa, 106 Pa or more, measured at shear rate 300 s⁻¹, under the following conditions:
   200 ml of soy milk supplemented with 2% glucose (as described in Example 2) are inoculated with 2 ml of an overnight culture of the lactic acid bacterium strain, and left at inoculation temperature until pH^{˜}4.55 (such as pH 4.49, 4.53, 4.54, 4.55 or 4.66), followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

In a preferred embodiment, the Lactococcus lactis lactic acid bacterium (LAB) strain provided in step (a) is a Lactococcus lactis lactic acid bacterium (LAB) strain comprising an active eps gene cluster capable of producing exopolysaccharide (EPS), wherein the eps gene cluster is selected from:
(i) a nucleotide sequence as defined in SEQ ID NO.: 1;
(ii) a nucleotide sequence as defined in SEQ ID NO.: 2;
(iii) a nucleotide sequence as defined in SEQ ID NO.: 3;
(iv) a nucleotide sequence as defined in SEQ ID NO.: 4; and
(v) a nucleotide sequence as defined in SEQ ID NO.: 5.

In a preferred embodiment, the Lactococcus lactis lactic acid bacterium (LAB) strain provided in step (a), which comprises a eps gene cluster as defined in SEQ ID NO.: 1, belongs to the MLST (multilocus sequence typing) group ST76, wherein the MLST analysis is performed as described in Example 4, i.e., with a 12 gene MLST scheme developed at Chr. Hansen. The scheme is based on the twelve genes dnaK, fusA, groEL, gyrA, gyrB, ileS, lepA, pheS, recA, rpoA, rpoB and rpoC chosen from the core genome of Lactobacillaceae (Salvetti et al., 2018). A total of 22493 bp are used in the scheme, which thereby represents almost 1% of the average Lactococcus genome. MLST typing with Illumina whole genome sequences is performed with the help of the CLC Microbial Genomics Module, which is a plugin to the CLC Genomics Workbench v10. In CLC, MLST is incorporated into Chr. Hansen's custom designed standard genome sequence analysis pipeline. It is performed both on de novo contigs and reference assemblies.

In a further preferred embodiment, the Lactococcus lactis lactic acid bacterium (LAB) strain provided in step (a), which comprises a eps gene cluster as defined in SEQ ID NO.: 2, or which comprises a eps gene cluster as defined in a nucleotide sequence which differs by no more than 1 nucleotide from the nucleotide sequence as defined in SEQ ID NO.: 2, belongs to the MLST (multilocus sequence typing) group ST76.

In a further preferred embodiment, the Lactococcus lactis lactic acid bacterium (LAB) strain provided in step (a), which comprises a eps gene cluster as defined in SEQ ID NO.: 3, or which comprises a eps gene cluster as defined in a nucleotide sequence which differs by no more than 1 nucleotide from the nucleotide sequence as defined in SEQ ID NO.: 3, belongs to the MLST (multilocus sequence typing) group ST76.

In a further preferred embodiment, the Lactococcus lactis lactic acid bacterium (LAB) strain provided in step (a), which comprises a eps gene cluster as defined in SEQ ID NO.: 4, or which comprises a eps gene cluster as defined in a nucleotide sequence which differs by no more than 1 nucleotide from the nucleotide sequence as defined in SEQ ID NO.: 4, belongs to the MLST (multilocus sequence typing) group ST76.

In a further preferred embodiment, the Lactococcus lactis lactic acid bacterium (LAB) strain provided in step (a), which comprises a eps gene cluster as defined in SEQ ID NO.: 5, or which comprises a eps gene cluster as defined in a nucleotide sequence which differs by no more than 5 nucleotides, preferably by no more than 4 nucleotides, more preferably by no more than 3 nucleotides, even more preferably by no more than 2 nucleotides, most preferably by no more than 1 nucleotide from the nucleotide sequence as defined in SEQ ID NO.: 5, belongs to the MLST (multilocus sequence typing) group ST140.

As discussed above, the MLST analysis is performed as described in Example 4, i.e., with a 12 gene MLST scheme developed at Chr. Hansen. The scheme is based on the twelve genes dnaK, fusA, groEL, gyrA, gyrB, ileS, lepA, pheS, recA, rpoA, rpoB and rpoC chosen from the core genome of Lactobacillaceae (Salvetti et al., 2018). A total of 22493 bp are used in the scheme, which thereby represents almost 1% of the average Lactococcus genome. MLST typing with Illumina whole genome sequences is performed with the help of the CLC Microbial Genomics Module, which is a plugin to the CLC Genomics Workbench v10. In CLC, MLST is incorporated into Chr. Hansen's custom designed standard genome sequence analysis pipeline. It is performed both on de novo contigs and reference assemblies.

In a further preferred embodiment, the Lactococcus lactis lactic acid bacterium (LAB) strain provided in step (a), which comprises a eps gene cluster as defined in SEQ ID NO.: 1 is able to generate fermented milks with a pH of about 4.55 in about 15 h or less (“time-to-pH-4.55” of 15 h or less), preferably in about 13 h or less (“time-to-pH 4.55” of 13 h or less), more preferably in about 12 h or less (“time-to-pH 4.55” of 12 h or less), even more preferably in about 11 h or less (“time-to-pH 4.55” of 11 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis lactic acid bacterium (LAB) strain provided in step (a), which comprises a eps gene cluster as defined in SEQ ID NO.: 1 is able to generate fermented milks with a pH of about 4.55 (such as a pH of about 4.49, 4.53 or 4.55) in about 21 h or less (“time-to-pH 4.55” of 21 h or less), preferably in about 16 h or less (“time-to-pH 4.55” of 16 h or less), more preferably in about 11 h or less (“time-to-pH 4.55” of 11 h or less), even more preferably in about 8 h or less (“time-to-pH 4.55” of 8 h or less), measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 (such as a pH of about 4.49, 4.53 or 4.55), as described above, is reached.

In a further preferred embodiment, the Lactococcus lactis lactic acid bacterium (LAB) strain provided in step (a), which comprises a eps gene cluster as defined in SEQ ID NO.: 2, or which comprises a eps gene cluster as defined in a nucleotide sequence which differs by no more than 1 nucleotide from the nucleotide sequence as defined in SEQ ID NO.: 2, is able to generate fermented milks with a pH of about 4.55 in about 15 h or less (“time-to-pH-4.55” of 15 h or less), preferably in about 13 h or less (“time-to-pH 4.55” of 13 h or less), more preferably in about 12 h or less (“time-to-pH 4.55” of 12 h or less), even more preferably in about 9 h or less (“time-to-pH 4.55” of 9 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis lactic acid bacterium (LAB) strain provided in step (a), which comprises a eps gene cluster as defined in SEQ ID NO.: 2, or which comprises a eps gene cluster as defined in a nucleotide sequence which differs by no more than 1 nucleotide from the nucleotide sequence as defined in SEQ ID NO.: 2, is able to generate fermented milks with a pH of about 4.55 (such as a pH of about 4.54, 4.55 or 4.66) in about 21 h or less (“time-to-pH 4.55” of 21 h or less), preferably in about 11 h or less (“time-to-pH 4.55” of 11 h or less), more preferably in about 10.5 h or less (“time-to-pH 4.55” of 10.5 h or less), measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 (such as a pH of about 4.54, 4.55 or 4.66), as described above, is reached.

In a further preferred embodiment, the Lactococcus lactis lactic acid bacterium (LAB) strain provided in step (a), which comprises a eps gene cluster as defined in SEQ ID NO.: 3, or which comprises a eps gene cluster as defined in a nucleotide sequence which differs by no more than 1 nucleotide from the nucleotide sequence as defined in SEQ ID NO.: 3, is able to generate fermented milks with a pH of about 4.55 in about 14 h or less (“time-to-pH-4.55” of 14 h or less), preferably in about 12 h or less (“time-to-pH 4.55” of 12 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis lactic acid bacterium (LAB) strain provided in step (a), which comprises a eps gene cluster as defined in SEQ ID NO.: 3, or which comprises a eps gene cluster as defined in a nucleotide sequence which differs by no more than 1 nucleotide from the nucleotide sequence as defined in SEQ ID NO.: 3, is able to generate fermented milks with a pH of about 4.55 in about 7.5 h or less (“time-to-pH 4.55” of 7.5 h or less, measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 is reached.

In a further preferred embodiment, the Lactococcus lactis lactic acid bacterium (LAB) strain provided in step (a), which comprises a eps gene cluster as defined in SEQ ID NO.: 4, or which comprises a eps gene cluster as defined in a nucleotide sequence which differs by no more than 1 nucleotide from the nucleotide sequence as defined in SEQ ID NO.: 4, is able to generate fermented milks with a pH of about 4.55 in about 13 h or less (“time-to-pH-4.55” of 13 h or less), preferably in about 11 h or less (“time-to-pH 4.55” of 11 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis lactic acid bacterium (LAB) strain provided in step (a), which comprises a eps gene cluster as defined in SEQ ID NO.: 4, or which comprises a eps gene cluster as defined in a nucleotide sequence which differs by no more than 1 nucleotide from the nucleotide sequence as defined in SEQ ID NO.: 4, is able to generate fermented milks with a pH of about 4.55 in about 10 h or less (“time-to-pH 4.55” of 10 h or less, measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 is reached.

In a further preferred embodiment, the Lactococcus lactis lactic acid bacterium (LAB) strain provided in step (a), which comprises a eps gene cluster as defined in SEQ ID NO.: 5, or which comprises a eps gene cluster as defined in a nucleotide sequence which differs by no more than 5 nucleotides, preferably by no more than 4 nucleotides, more preferably by no more than 3 nucleotides, even more preferably by no more than 2 nucleotides, most preferably by no more than 1 nucleotide from the nucleotide sequence as defined in SEQ ID NO.: 5, is able to generate fermented milks with a pH of about 4.55 in about 10 h or less (“time-to-pH-4.55” of 10 h or less), preferably in about 8 h or less (“time-to-pH 4.55” of 8 h or less), measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until the pH 4.55 is reached.

In a further preferred embodiment, the Lactococcus lactis lactic acid bacterium (LAB) strain provided in step (a), which comprises a eps gene cluster as defined in SEQ ID NO.: 5, or which comprises a eps gene cluster as defined in a nucleotide sequence which differs by no more than 5 nucleotides, preferably by no more than 4 nucleotides, more preferably by no more than 3 nucleotides, even more preferably by no more than 2 nucleotides, most preferably by no more than 1 nucleotide from the nucleotide sequence as defined in SEQ ID NO.: 5, is able to generate fermented milks with a pH of about 4.55 in about 10.5 h or less (“time-to-pH 4.55” of 10.5 h or less, measured under the following conditions:

1% volume (2 ml) overnight microbial culture (obtained by inoculating the microbial culture in M17 broth supplemented with 2% glucose at 30° C.) is inoculated in 200 ml soy milk with glucose, such as 0.5-5% glucose, preferably 0.5-2% glucose, more preferably about 2% glucose and left at inoculation temperature (30° C.) until the pH of about 4.55 is reached.

The skilled person is aware of means to provide a Lactococcus lactis lactic acid bacterium (LAB) strain according to step a). For instance, strains can be isolated from different sources, and the eps gene cluster can be sequenced my means known in the art. In addition, mutants can be obtained, and the eps gene cluster of the mutants can be sequenced my means known in the art. Lactococcus lactis lactic acid bacterium (LAB) strains according to step a) can also be provided by genetic engineering. Oligonucleotides carrying the desired eps gene cluster may be used to amplify a specific DNA fragment by PCR. The PCR fragment carrying the desired sequence is cloned into a vector plasmid and transformed into another lactic acid bacterium (LAB) target strain.

Any combination of the above-described elements, aspects and embodiments in all possible variations thereof is encompassed by the invention unless otherwise indicated herein or otherwise clearly contradicted by context. Embodiments of the present invention are described below, by way of examples only.

### EXAMPLES

### Example 1. High-Throughput Screening for Texturing Strains and Measurement of Milk Gel Texture

L. lactis is used to produce numerous fermented dairy products including cheese and mesophilic fermented milk, such as buttermilk and sour cream. Polysaccharide-producing strains are of great interest for these applications, as polysaccharides released into the medium can result in improved texturing properties of buttermilk and sour cream, while capsular polysaccharides can result in improved water-holding capacity and thus improved yields of, e.g., cheese.

Milk (liquid) is typically converted into milk gel (soft solid) when fermented with lactic acid bacteria typically belonging to, e.g., Streptococcus thermophilus, Lactobacillus spp. and Lactococcus lactis spp. Rheometer or texture analyzer are typically used to assess rheological properties of fermented milk gels, such as shear stress. Shear stress measurements are related to perceived mouth thickness, when the texture of milk gels is assessed by a sensory panel. High mouth thickness is considered an important quality factor of fermented milk gels such as yoghurt, and consumer acceptance is often very closely linked to the texture properties such as mouth thickness, which is a function of shear stress.

A liquid handling unit, Hamilton Robotics MicroLab Star, equipped with pressure sensor inside the air displacement barrel of the individual pipettes was used to screen for texturing strains as described in Poulsen et al., 2019. The liquid handler has a pressure sensor located in the headspace of each pipetting channel. Pressure data from each sensor was collected by TADM (Total Aspiration Dispense Monitoring) software of the Hamilton Robotics MicroLab Star liquid handler (Hamilton Robotics) and used to assess the relative shear stress of milk gel samples.

L. lactis from high-throughput screening strain library were screened for texturing properties using the TADM tool of the Hamilton liquid handling robot, as stated above, in 2-ml scale. Pressure versus time data (TADM) were obtained from 2-ml samples made in a 96-well micro-titer plate, where B-milk was inoculated for 20 h at 30° C. in the presence of different strains (1% of inoculum) unless otherwise stated, and then stored at 4° C. for 1 day. Hamilton liquid handling unit was used to measure pressure during aspiration, and the area above the pressure curves obtained during aspiration was used to compare the texturing abilities of the strains.

Shear stress data were obtained by inoculating the same microbial cultures in semi-fat milk (1.5% fat); milk was heated at 90° C. for 20 min and cooled down to the inoculation temperature, prior to inoculation with 1% volume overnight microbial culture. The inoculation took place for 8-22 h at 30° C. in 200-ml scale until pH^{˜}4.55, followed by cooling to 4° C. and storage until shear stress is measured, typically from 1-7 days, such as for 5 days at 4° C. After the storage, the fermented milk was stirred gently by means of a stick fitted with a bored disc until homogeneity of the sample. Shear stress of the samples was assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

The good texturing ability of 22 new strains DSM 33193, 33226, 33194, 33133, 33195, 33196, 33197, 33200, 33201, 33202, 33203, 33204, 33205, 33218, 33219, 33220, 33221, 33222, 33223, 33224, 33225 and 33192 was confirmed using the rheometer as described above. Further, the good texturing ability of strain DSM 25485 was also confirmed using the rheometer as described above. The results are shown in Table 1.

TABLE 1
Shear stress and time-to-pH 4.55 for selected texturing strains (1% of inoculum) ±
yeast extract (0.2%, see below for the details) ± potential co-acidifier (DSM 24649 or DSM
25485) incubated in heat-treated semi-fat milk at 30° C. Some of the strains under
investigation are slow acidifiers and require either presence of yeast extract or a co-acidifier
strain to acidify milk in an acceptable time, e.g., about 13 h or less to reach a pH of 4.55.
Strains DSM 24649 and DSM 25485 were used alone or in combination with strains under
investigation and/or as potential co-acidifiers (10% inoculum - which corresponds to a ratio
9:1 (selected texturing strain:co-acidifier strain)). Generally, 1% inoculum of the strains in
milk (i.e. 2 ml overnight inoculum in M17 medium added to 200 ml milk) was used. When a
single strain is used as inoculum, 2 ml (100%) of this strain are added to 200 ml milk (1%
inoculum in milk). When a strain is used together with a co-acidifier, 90% of the strain under
investigation (1.8 ml) and 10% (0.2 ml) of a co-acidifier are used (10% of inoculum for the
co- acidifier, ratio 9:1 (selected texturing strain:co-acidifier strain)).

| | Shear stress | | | | Time-to-pH 4.55, h | | | | |
|---|---|---|---|---|---|---|---|---|---|
| | | + | + | + | | + | + | + | |
| Deposit no. | alone | 24649 | 25485 | YE | alone | 24649 | 25485 | YE | MLST |
| DSM 24649 | 20 | | | | 8 | | | | ST45 |
| DSM 25485 | 56 | | | | 12 | | | | ST76 |
| DSM 33192 | 94 | | | 88 | 14 | | | 12 | ST47 |
| DSM 33193 | 86 | | | | 11 | | | | ST76 |
| DSM 33226 | | 64 | 98 | | | 14 | | | ST76 |
| DSM 33194 | | 58 | 95 | 83 | | 15 | | | ST76 |
| DSM 33133 | 75 | | | 69 | 8 | | | 7 | ST140 |
| DSM 33195 | 88 | 30 | 80 | | 22 | 10 | 19 | | ST76 |
| DSM 33196 | 86 | | | | 11 | | | | ST76 |
| DSM 33197 | 121 | | | 90 | 12 | | | 8 | ST76 |
| DSM 33200 | 66 | | | | 9 | | | | ST76 |
| DSM 33201 | 62 | | | | 12 | | | | ST76 |
| DSM 33202 | 51 | | | | 16 | | | | ST76 |
| DSM 33203 | 65 | | | | 13 | | | | ST76 |
| DSM 33204 | 95 | | | | 13 | | | | ST76 |
| DSM 33205 | 61 | | | | 12 | | | | ST76 |
| DSM 33218 | 69 | | | | 11 | | | | ST76 |
| DSM 33219 | 87 | | | | 13 | | | | ST76 |
| DSM 33220 | 89 | | | | 12 | | | | ST76 |
| DSM 33221 | 70 | | | | 11 | | | | ST76 |
| DSM 33222 | 64 | | | | 12 | | | | ST76 |
| DSM 33223 | 60 | | | | 18 | | | | ST76 |
| DSM 33224 | 67 | | | | 12 | | | | ST76 |
| DSM 33225 | 69 | | | | 11 | | | | ST76 |

Since an enhanced texture is associated with the production of polysaccharides, mining for eps gene clusters was performed. The eps gene clusters are typically chromosomal in L. lactis subsp. lactis but can reside on a plasmid in L. lactis subsp. cremoris (Poulsen et al. 2019). Generally, eps gene clusters are highly diverse, and their nucleotide sequences are among the most variable sequences in LAB genomes. Also, the structural variety of polysaccharide molecules is enormous. The type and size of polysaccharides and their interaction with milk proteins is the determining factor for texture development.

All of the 22 strains (DSM 33193, 33226, 33194, 33133, 33195, 33196, 33197, 33200, 33201, 33202, 33203, 33204, 33205, 33218, 33219, 33220, 33221, 33222, 33223, 33224, 33225 and 33192) have eps gene clusters similar to that of NIZO B40 strain (FIG. 1). As the eps cluster in L. lactis subsp. cremoris NIZO B40 is known to be situated on a plasmid, it is likely also situated on a plasmid in the strains mentioned.

In addition, it is believed that the above strains can differ in their genome content and may be different phenotypically, see Example 5.

### Example 2. Rheology Measurement of Texture in Soy Milk

Rheology measurements of 22 L. lactis strains in soy milk supplemented with 2% glucose were performed.

The strains tested in this example were the following: DSM 24649; DSM 25485; DSM 33192; DSM 33193; DSM 33226; DSM 33194; DSM 33133; DSM 33195; DSM 33196; DSM 33197; DSM 33200; DSM 33201; DSM 33202; DSM 33203; DSM 33204; DSM 33205; DSM 33218; DSM 33219; DSM 33220; DSM 33221; DSM 33222; DSM 33223; DSM 33224; and DSM 33225.

The milk base used in was soy milk supplemented with 2% glucose: The soy milk was organic and unsweetened, obtained from Naturli' Foods, with the following composition per 100 ml:

Fat: 2.1 g—Thereof saturated fat: 0.4 g

Carbohydrates: 0.1 g—Thereof sugars: 0.1 g

Fibers: 0.6 g

Protein: 3.7 g

Salt: 0.04 g.

The milk was already sterile, and no pre-treatment was performed to it before its use. It was supplemented with glucose 2%.

1% volume overnight microbial culture (obtained by inoculating the microbial cultures in M17 broth supplemented with 2% glucose at 30° C.) was inoculated in soy milk with 2% glucose. The inoculation took place at 30° C. in 200-ml scale until pH^{˜}4.55 (see Table 3 for the specific pH reached by each culture), followed by cooling to 4° C. and storage until shear stress is measured, typically from 1-7 days, such as for 5 days. After the storage, the fermented milk was stirred gently by means of a stick fitted with a bored disc until homogeneity of the sample. Shear stress of the samples was assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

The results of the shear stress (Pa) are shown in Table 2 below. “Alpro” refers to “Alpro naturell mild & creamy plantgurt”, a commercially-available fermented soy milk from “Alpro” (https://www.alpro.com/se/produkter/vaxtbaserad-yoghurt-variant/mild-creamy/mild-creamy-naturell/), with the following composition per 100 ml:

| Energy | 210 kJ/50 kcal | |
|---|---|---|
| Fat | 2.3 | g |
| Saturated fatty acids | 0.4 | g |
| Monounsaturated | 0.5 | g |
| Polyunsaturated | 1.4 | g |
| Carbohydrates | 2.1 | g |
| Sugar | 2.1 | g |
| Fibers | l | g |
| Protein | 4 | g |
| Salt | 0.3 | g |
| Vitamins | | |
| Vitamin D | 0.75 | μg |
| Vitamin B12 | 0.38 | μg |
| Minerals | | |
| Calcium | 120 | mg |

And with the following ingredients: Water, peeled SOYBEANS (7.9%), sugar, tricalcium citrate, stabilizer (pectin), acidity regulators (sodium citrate, citric acid), sea salt, antioxidants (tocopherol-rich extract, ascorbic acid esters of edible fatty acids), vitamins (B12, D2), yogurt culture (S. thermophilus, L. bulgaricus). Of note, Alpro comprises pectin, which increases the texture of the fermented milk. However, the base milk used in this example (soy milk supplemented with 2% glucose) does not include pectin.

TABLE 2
Shear stress (Pa) at several shear rates (s⁻¹) for selected texturing strains
(1% of inoculum) incubated in soy milk without 2% glucose, as described above.

| | Shear stress (Pa) at shear rate (s⁻¹) | | | | | | | | | | | | | | | | | | | | |
|---|---|---|---|---|---|---|---|---|---|---|---|---|---|---|---|---|---|---|---|---|---|
| Sample (DSM) | 0, 27 | 15 | 30 | 45 | 60 | 75 | 90 | 105 | 120 | 135 | 150 | 165 | 180 | 195 | 210 | 225 | 240 | 255 | 270 | 285 | 300 |
| Alpro | 17 | 36 | 40 | 42 | 44 | 46 | 47 | 48 | 49 | 50 | 51 | 52 | 53 | 54 | 55 | 56 | 56 | 57 | 58 | 60 | 60 |
| DSM 24649 | 7 | 16 | 18 | 19 | 20 | 20 | 21 | 21 | 21 | 21 | 22 | 22 | 22 | 23 | 23 | 23 | 23 | 24 | 24 | 24 | 24 |
| DSM 33193 | 7 | 40 | 57 | 69 | 78 | 86 | 92 | 98 | 102 | 105 | 107 | 109 | 110 | 110 | 110 | 110 | 109 | 109 | 108 | 107 | 106 |
| DSM 33226 | 5 | 25 | 37 | 47 | 55 | 61 | 66 | 71 | 74 | 77 | 79 | 81 | 82 | 83 | 84 | 85 | 85 | 85 | 85 | 85 | 86 |
| DSM 33194 | 6 | 27 | 43 | 54 | 63 | 70 | 75 | 80 | 84 | 87 | 89 | 91 | 92 | 93 | 93 | 93 | 93 | 93 | 93 | 93 | 93 |
| DSM 33133 | 5 | 15 | 21 | 27 | 32 | 36 | 39 | 42 | 45 | 47 | 49 | 50 | 51 | 52 | 53 | 54 | 55 | 55 | 56 | 56 | 57 |
| DSM 33195 | 3 | 19 | 34 | 46 | 55 | 63 | 69 | 73 | 76 | 79 | 80 | 81 | 82 | 82 | 82 | 82 | 81 | 81 | 80 | 80 | 79 |
| DSM 33196 | 6 | 28 | 45 | 56 | 66 | 74 | 80 | 86 | 90 | 93 | 95 | 97 | 98 | 99 | 99 | 99 | 99 | 99 | 99 | 99 | 99 |
| DSM 33197 | 5 | 30 | 44 | 55 | 63 | 71 | 77 | 82 | 86 | 89 | 91 | 93 | 95 | 96 | 97 | 97 | 97 | 97 | 97 | 97 | 96 |
| DSM 33200 | 4 | 15 | 18 | 20 | 23 | 24 | 26 | 27 | 28 | 29 | 30 | 31 | 31 | 32 | 32 | 33 | 33 | 34 | 34 | 35 | 35 |
| DSM 33201 | 4 | 15 | 19 | 23 | 26 | 29 | 31 | 33 | 35 | 36 | 37 | 38 | 39 | 40 | 41 | 42 | 43 | 43 | 44 | 45 | 45 |
| DSM 33202 | 5 | 18 | 21 | 23 | 25 | 27 | 28 | 29 | 30 | 31 | 31 | 32 | 32 | 33 | 33 | 33 | 34 | 34 | 35 | 36 | 36 |
| DSM 33203 | 5 | 18 | 25 | 33 | 38 | 43 | 46 | 50 | 52 | 54 | 56 | 58 | 59 | 60 | 61 | 62 | 62 | 62 | 63 | 64 | 64 |
| DSM 33204 | 6 | 24 | 38 | 49 | 58 | 65 | 71 | 75 | 79 | 81 | 84 | 85 | 86 | 87 | 88 | 88 | 88 | 88 | 88 | 88 | 88 |
| DSM 33205 | 6 | 23 | 35 | 46 | 53 | 58 | 62 | 66 | 69 | 71 | 72 | 73 | 74 | 75 | 75 | 75 | 75 | 75 | 75 | 75 | 74 |
| DSM 33218 | 4 | 16 | 21 | 26 | 31 | 35 | 39 | 42 | 45 | 47 | 49 | 50 | 51 | 52 | 53 | 54 | 54 | 55 | 56 | 56 | 56 |
| DSM 33219 | 5 | 16 | 23 | 31 | 38 | 44 | 49 | 54 | 58 | 61 | 64 | 66 | 68 | 70 | 71 | 72 | 73 | 74 | 74 | 75 | 75 |
| DSM 33220 | 6 | 37 | 56 | 68 | 77 | 84 | 90 | 94 | 98 | 100 | 102 | 103 | 104 | 104 | 104 | 104 | 103 | 103 | 102 | 102 | 102 |
| DSM 33221 | 5 | 18 | 27 | 35 | 39 | 43 | 47 | 50 | 52 | 54 | 56 | 57 | 58 | 59 | 60 | 60 | 61 | 61 | 61 | 61 | 62 |
| DSM 33222 | 5 | 16 | 22 | 29 | 35 | 40 | 44 | 47 | 50 | 53 | 55 | 57 | 58 | 59 | 60 | 61 | 62 | 62 | 63 | 63 | 63 |
| DSM 33223 | 9 | 33 | 41 | 46 | 50 | 54 | 57 | 60 | 62 | 64 | 65 | 67 | 68 | 69 | 70 | 70 | 70 | 71 | 71 | 71 | 71 |
| DSM 33224 | 5 | 16 | 21 | 27 | 33 | 38 | 43 | 46 | 50 | 52 | 54 | 56 | 57 | 59 | 60 | 60 | 61 | 62 | 62 | 62 | 63 |
| DSM 33225 | 4 | 16 | 20 | 24 | 29 | 33 | 37 | 40 | 43 | 45 | 47 | 49 | 50 | 51 | 52 | 53 | 54 | 55 | 55 | 56 | 56 |
| DSM 25485 | 6 | 17 | 25 | 30 | 34 | 38 | 41 | 43 | 45 | 47 | 48 | 49 | 50 | 51 | 52 | 52 | 53 | 53 | 54 | 54 | 54 |
| DSM 33192 | 5 | 16 | 21 | 25 | 29 | 32 | 35 | 37 | 39 | 40 | 41 | 42 | 43 | 43 | 44 | 45 | 46 | 46 | 46 | 47 | 47 |

As it can be seen from Tables 2, all selected texturing strains showed higher shear stress when fermenting soy milk supplemented with glucose 2% than the negative control (DSM 24649). In addition, strains DSM 33221, DSM 33224, DSM 33222, DSM 33203, DSM 33223, DSM 33205, DSM 33219, DSM 33195, DSM 33226, DSM 33204, DSM 33194, DSM 33197, DSM 33196, DSM 33220 and DSM 33193 showed higher shear stress than the commercially available fermented soy product, “Alpro”, which comprises pectin, as described above.

Finally, the pH reached by each of the strains, and the time to this pH (in h) is shown in Table 3.

TABLE 3
Time-to-pH for selected texturing strains (1% of inoculum) incubated in
soy milk supplemented with 2% glucose, as described above.

| Strain | pH reached | Time to pH, h |
|---|---|---|
| Alpro | | |
| DSM 24649 | 4, 55 | 8 |
| DSM 33193 | 4, 55 | 14 |
| DSM 33226 | 4, 55 | 11 |
| DSM 33194 | 4, 55 | 13, 5 |
| DSM 33133 | 4, 55 | 10, 5 |
| DSM 33195 | 4, 55 | 13, 5 |
| DSM 33196 | 4, 55 | 14 |
| DSM 33197 | 4, 55 | 15 |
| DSM 33200 | 4, 54 | 10, 5 |
| DSM 33201 | 4, 55 | 11 |
| DSM 33202 | 4, 66 | 21 |
| DSM 33203 | 4, 55 | 10, 5 |
| DSM 33204 | 4, 55 | 14 |
| DSM 33205 | 4, 55 | 8 |
| DSM 33218 | 4, 49 | 7, 5 |
| DSM 33219 | 4, 55 | 16 |
| DSM 33220 | 4, 55 | 14, 5 |
| DSM 33221 | 4, 53 | 10 |
| DSM 33222 | 4, 55 | 7, 5 |
| DSM 33223 | 4, 55 | 21 |
| DSM 33224 | 4, 55 | 8 |
| DSM 33225 | 4, 55 | 10 |
| DSM 25485 | 4, 55 | 16 |
| DSM 33192 | 4, 55 | 14 |

### Example 3. Sequencing Genomes of Lactococcal Strains and Characterization of their Eps Gene Clusters

The genome of the strains was sequenced in-house at Chr. Hansen as described by Agersø et al. (Agersoe et al., 2018). In brief, total DNA was purified and used to prepare a 250-bp paired-end library for genome sequencing using Illumina MiSeq system. The sequence reads were subjected to quality trimming (Phred score<25) and assembled into contigs using the de novo assembly algorithm in CLC Genomics Workbench, version 10.1.1 (CLC bio, Qiagen Bioinformatics). The resulting genome assembly was filtered by removing contigs with coverage of <15× and/or <20% of the median coverage of the assembly. The consensus sequence of the remaining contigs was exported in FASTA format, which is referred to as the draft genome sequence, and used in the subsequent sequence analysis.

The below Table 4 shows the percent identity matrix of the eps gene cluster of the above strains. Multiple Sequence Alignment tool Clustal Omega (Clustal2.1) (https://www.ebi.ac.uk/Tools/msa/clustalo/) with standard parameters was used to assess the sequence identity (on nucleotide level) of the eps gene cluster of the strains.

TABLE 4
Identity matrix. The identity between two strains is expressed as a number
(in %) found on the cross between the corresponding strains (identified
by their DSM accession number), one in a row and another in a column.

| | | | | | | 33192, 33193, 33226, |
|---|---|---|---|---|---|---|
| | | | | | | 33194, 33195, 33196, 33197, |
| | | | 33200, 33201, | | | 33204, 33205, 33218, 33219, |
| | 33133 | 33222 | 33202, 33203 | 33225 | B40 | 33220, 33221, 33223, 33224 |
| 33133 | 100 | 99, 95 | 99, 95 | 99, 95 | 99, 95 | 99, 96 |
| 33222 | 99, 95 | 100 | 99, 98 | 99, 98 | 99, 98 | 99, 99 |
| 33200, 33201, 33202, 33203 | 99, 95 | 99, 98 | 100 | 99, 98 | 99, 98 | 99, 99 |
| 33225 | 99, 95 | 99, 98 | 99, 98 | 100 | 99, 98 | 99, 99 |
| B40 | 99, 95 | 99, 98 | 99, 98 | 99, 98 | 100 | 99, 99 |
| 33192, 33193, 33226, | 99, 96 | 99, 99 | 99, 99 | 99, 99 | 99, 99 | 100 |
| 33194, 33195, 33196, 33197, | | | | | | |
| 33204, 33205, 33218, 33219, | | | | | | |
| 33220, 33221, 33223, 33224 | | | | | | |

The NIZO B40 strain harbors a 42,180 bp EPS-plasmid pNZ4000 (van Kranenburg et al., 2000), containing the 12 kb NIZO B40 eps gene cluster with 14 coordinately expressed genes. This strain produces a polymer with the following repeating unit: →4)[α-L-Rhap-(1→2)][α-D-Galp-1-PO4-3]-β-D-Galp-(1→4)-β-D-Glcp-(1→4)-β-D-Glcp-(1→(van Casteren et al., 1998):

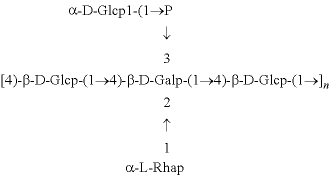

The functionality of the genes of the eps gene cluster of L. lactis NIZO B40 is reviewed in Kleerebezem et al., 2002 and van Kranenburg et al., 1999. Based on sequence comparisons, putative functions could be assigned to several eps genes, predicting their involvement in biosynthesis of the repeating unit oligosaccharides by the sequential addition of sugars to a membrane-anchored lipid carrier, and subsequent export (by wzx) and polymerisation (by wzy) of these lipid-linked oligosaccharides. EpsE links glucose-1-phosphate from UDP-glucose to a lipid carrier, GT1 and GT2 link glucose from UDP-glucose to lipid-linked glucose, and GT3 links galactose from UDP-galactose to lipid-linked cellobiose. The protein containing Glyphos_trans domain (pfam04464) is a phosphotransferase appeared to be involved in EPS biosynthesis as a galactosyl phosphotransferase or an enzyme which releases the backbone oligosaccharide from the lipid carrier. GT4 seems involved in attaching the rhamnose to the B40-EPS repeating unit (Kleerebezem et al, 2002; van Kranenburg et al 1999).

The eps gene cluster of strains DSM 33193, 33226, 33194, 33133, 33195, 33196, 33197, 33200, 33201, 33202, 33203, 33204, 33205, 33218, 33219, 33220, 33221, 33222, 33223, 33224, 33225 and 33192 is similar but not identical to the one of L. lactis NIZO B40. It includes 15 open reading frames (ORF) and oriented in the same transcriptional sense except for the last gene of the cluster, lytR (FIG. 1). As shown in the identity matrix, the eps gene clusters are similar but not identical to the eps gene cluster of strain NIZO B-40. At least one single nucleotide polymorphism was found in NIZO B40 strain compared to the strains DSM 33193, 33226, 33194, 33133, 33195, 33196, 33197, 33200, 33201, 33202, 33203, 33204, 33205, 33218, 33219, 33220, 33221, 33222, 33223, 33224, 33225 and 33192. Nucleotide 1276 is G in B40 and A in all Chr. Hansen's strains. It results in amino acid change in the protein EpsC from the basic amino acid arginine (R, represented by CGA) in NIZO B40 to polar amino acid glutamine (Q, represented by CAA) in Chr. Hansen's strains. The amino acid change can have an influence on e.g. the amount of EPS produced, which, in turn, may have an influence on the texture of the milk fermented with these strains.

Eps genes with the same names often have different functions in different organisms, as the genes are often designated alphabetically by order of occurrence in a given locus and not based on their functions (for a review, see Zeidan et al., 2017). In NIZO strain B40, the EPS polymeraze is named epsI, and the export gene, epsK (e.g. van Kranenburg et al., 2000), while in SMQ-461, the genes with corresponding functions are names epsH and epsM. We have named the conserved genes of the strains according to the nomenclature suggested by Zeidan et al. (2017) (FIG. 1). In this patent application, the polymerase and the flippase genes are named wzy and wzx, respectively, while the genes epsABCD in van Kranenburg et al. (2000) correspond to epsCDBE in Poulsen et al. (2019) and in this patent application.

### Example 4. MLST (Multilocus Sequence Typing) Analysis

MLST (multilocus sequence typing) analysis and fingerprinting were used to investigate if the strains containing the NIZO B40-like eps gene clusters resemble each other in the remaining part of the genome.

Lactococci strains were typed with a 12 gene MLST scheme developed at Chr. Hansen. The scheme is based on the twelve genes dnaK, fusA, groEL, gyrA, gyrB, ileS, lepA, pheS, recA, rpoA, rpoB and rpoCchosen from the core genome of Lactobacillaceae (Salvetti et al., 2018). A total of 22493 bp were used in the scheme, which thereby represents almost 1% of the average Lactococcus genome. MLST typing with Illumina whole genome sequences was performed with the help of the CLC Microbial Genomics Module, which is a plugin to the CLC Genomics Workbench v10. In CLC, MLST is incorporated into our custom designed standard genome sequence analysis pipeline. It is performed both on de novo contigs and reference assemblies.

The results of the analysis are shown in Table 1. Despite strains DSM 33193, 33226, 33194, 33195, 33196, 33197, 33201, 33203, 33204, 33205, 33218, 33219, 33220, 33221, 33222, 33223, 33224 and 33225 belong to the same MLST group as NIZO-B40 (ST76), they do not all have the same phenotype, based on their texture (recorded as shear stress) and acidification speed (“time-to-pH 4.55”) (see Table 1). In addition, as shown in Example 5 below, there are further differences among the strains.

### Example 5. Strain Phenotypic Characterization

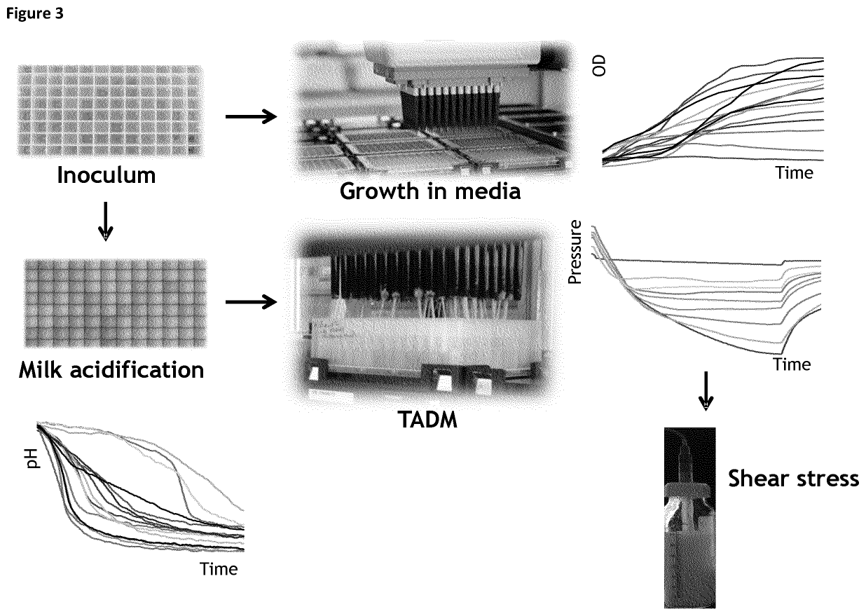

The strains have been characterized in several different assays. Experimental setup for characterization of strains for their texturing, milk acidification properties, and media (e.g. C source) preferences in 96-well micro-titer plate format are shown in FIG. 3. Milk acidification descriptors extracted from the acidification curves in 2-ml and 0.2-ml micro-titer plate scale, such as minimal pH and V max, were used to compare between the ability of the strains to acidify B-milk. Milk acidification in the presence of yeast extract was performed by performing milk acidifications in B-milk containing 0.2% yeast extract. To evaluate the ability of strains to tolerate salt, which is a relevant characteristic if the strains are to be used for cheese production, the strains were incubated in B-milk at 30° C. for 2 h, and then 50 μl were transferred to another micro-titer plate containing 150 μl milk±4% salt. The ability of the strains to use different carbon sources was evaluated by growing the strains in M17 medium containing 2% of different carbon sources, such as lactose, glucose, sucrose, fructose, galactose, xylose, maltose, trehalose (1%) or using water as control. To evaluate if the strains are tolerant to nisin, which is an antibacterial peptide produced by some L. lactis strains and used as food preservative, the strains were measured for their capacity to acidify B-milk in the presence and absence of nisin (0.2 mg/ml Chrisin corresponding to 4.52 μg/ml nisin). Nisin-tolerant strains are defined as strains with acidification curves not affected by addition of nisin as compared to acidification without nisin. For these strains, the lag-time and the slope of the milk acidification curve is similar in the presence and absence of nisin. To evaluate if the strains are resistant or susceptible to phages, a mixture of 50 diverse phages isolated from different host strains covering Chr. Hansen production L. lactis strains was added to milk. If the milk acidification curves are not affected by the presence of the phage pool and are the same with and without the phage presence, the strains are considered phage resistant.

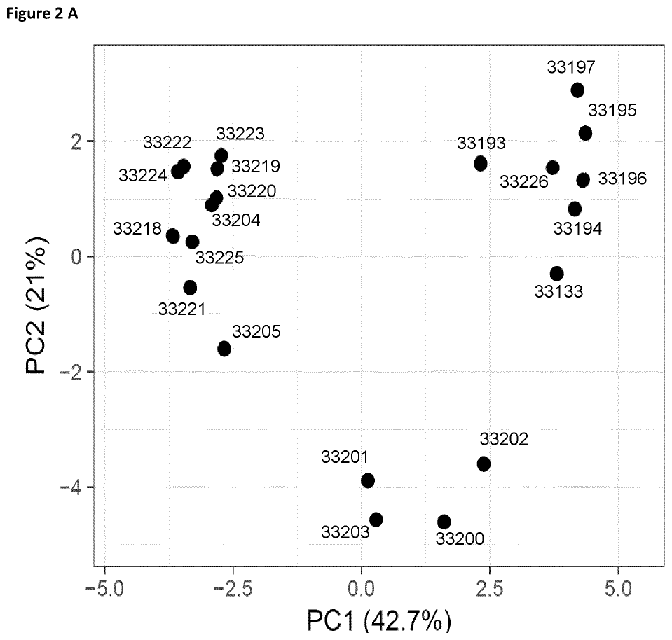

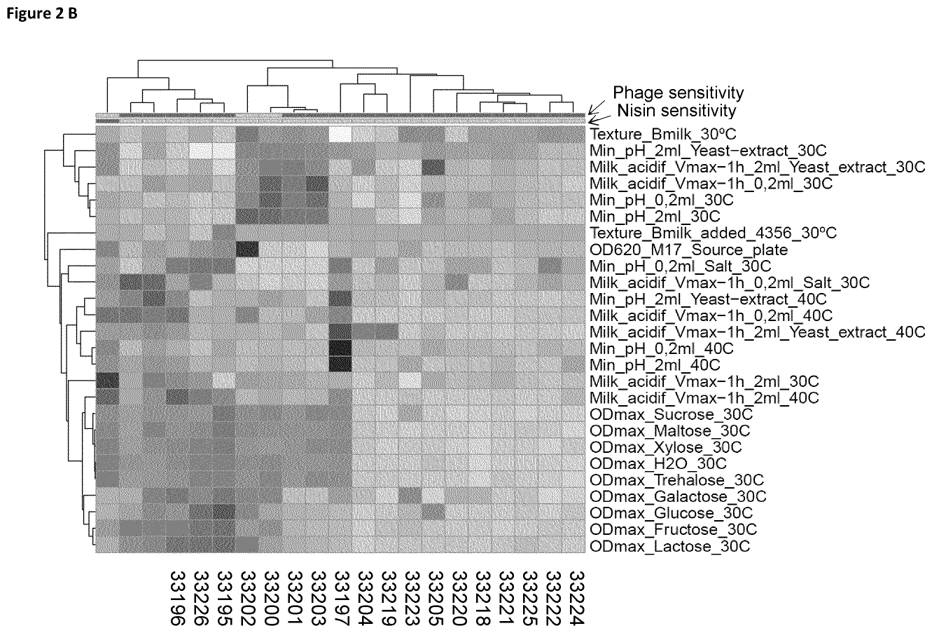

FIG. 2 shows differences between the novel texturing strains. Principal Component Analysis (PCA) was used to reduce the dimensionality of a large dataset, so that each strain (for which we have measured several parameters) was represented by one point. Strains situated close to each other on the PCA plot (FIG. 2A) are similar, based on their phenotypic characteristics. The data used for the PCA plot construction are visualized in FIG. 2B. As it can be seen in FIG. 2A and FIG. 2B, the strains are not identical among them. For instance, strains DSM 33133, 33200 and 33202 were found to be resistant to phages, while the remaining strains are sensitive to a pool of 50 phages used for the analysis. DSM 33133 was found to be tolerant to nisin, while the remaining strains from FIG. 2 were sensitive. Moreover, DSM 33133 has a different genetic background compared to the remaining strains from FIG. 2, as it belongs to a different MLST group (Table 1).

### Deposit and Expert Solution

The Applicant requests that a sample of the deposited microorganisms stated below may only be made available to an expert, until the date on which the patent is granted. In particular, the Applicant requests that the availability of the deposited microorganism referred to in Rule 33 EPC shall be effected only by the issue of a sample to an independent expert nominated by the requester (Rule 32(1) EPC).

TABLE 5
Deposits made by the Applicant, CHR. HANSEN A/S, at a Depositary Institution
having acquired the status of International Depositary Authority under the Budapest Treaty
on the International Recognition of the Deposit of Microorganisms for the Purposes of
Patent Procedure: Leibniz Institute DSMZ-German Collection of Microorganisms and Cell
Cultures Inhoffenstr. 7B, 38124 Braunschweig, Germany.

| Strain | Accession No. | Deposit date |
|---|---|---|
| Lactococcus lactis subsp. cremoris | DSM 25485 | Dec. 14, 2011 |
| Lactococcus lactis subsp. cremoris | DSM 24649 | Mar. 15, 2011 |
| Lactococcus lactis subsp. lactis | DSM 29291 | Aug. 21, 2014 |
| Lactococcus lactis | DSM 33137 | May 28, 2019 |
| Lactococcus lactis | DSM 33139 | May 28, 2019 |
| Lactococcus lactis | DSM 33140 | May 28, 2019 |
| Lactococcus lactis | DSM 33192 | Jul. 3, 2019 |
| Lactococcus lactis | DSM 33142 | May 28, 2019 |
| Lactococcus lactis | DSM 33201 | Jul. 3, 2019 |
| Lactococcus lactis | DSM 33133 | May 28, 2019 |
| Lactococcus lactis | DSM 33225 | Jul. 3, 2019 |
| Lactococcus lactis | DSM 33222 | Jul. 3, 2019 |
| Lactococcus lactis | DSM 33203 | Jul. 3, 2019 |
| Lactococcus lactis | DSM 33200 | Jul. 3, 2019 |
| Lactococcus lactis | DSM 33193 | Jul. 3, 2019 |
| Lactococcus lactis | DSM 33196 | Jul. 3, 2019 |
| Lactococcus lactis | DSM 33197 | Jul. 3, 2019 |
| Lactococcus lactis | DSM 33204 | Jul. 3, 2019 |
| Lactococcus lactis | DSM 33205 | Jul. 3, 2019 |
| Lactococcus lactis | DSM 33220 | Jul. 3, 2019 |
| Lactococcus lactis | DSM 33221 | Jul. 3, 2019 |
| Lactococcus lactis | DSM 33218 | Jul. 3, 2019 |
| Lactococcus lactis | DSM 33219 | Jul. 3, 2019 |
| Lactococcus lactis | DSM 33224 | Jul. 3, 2019 |
| Lactococcus lactis | DSM 33202 | Jul. 3, 2019 |
| Lactococcus lactis | DSM 33195 | Jul. 3, 2019 |
| Lactococcus lactis | DSM 33194 | Jul. 3, 2019 |
| Lactococcus lactis | DSM 33226 | Jul. 3, 2019 |
| Lactococcus lactis | DSM 33223 | Jul. 3, 2019 |

### REFERENCES

1. Zeidan et al., 2017, Polysaccharide production by lactic acid bacteria: from genes to industrial applications. FEMS Microbiol Rev 41: 168-200.
2. Bentley S. D. et al., 2006, Genetic analysis of the capsular biosynthetic locus from all 90 pneumococcal serotypes, PLoS Genet. 2:e31.
3. Dabour N., LaPointe G., 2005, Identification and Molecular Characterization of the Chromosomal Exopolysaccharide Biosynthesis Gene Cluster from Lactococcus lactis subsp. cremoris SMQ-461, Appl Environ Microbiol., 71: 7414-7425.
4. Nierop Groot M. N., Kleerebezem M., 2007, Mutational analysis of the Lactococcus lactis NIZO B40 exopolysaccharide (EPS) gene cluster: EPS biosynthesis correlates with unphosphorylated EpsB, J Appl Microbiol., 103: 2645-2656.
5. Pan D., Mei X. 2010, Antioxidant activity of an exopolysaccharide purified from Lactococcus lactis subsp. lactis 12, Carbohydrate Polymers, 80: 908-914.
6. Savijoki, K., et al., 2006, Proteolytic systems of lactic acid bacteria, Appl Microbiol Biotechnol, 71: 394-406
7. Suzuki C. et al., 2013, Novel exopolysaccharides produced by Lactococcus lactis subsp. lactis, and the diversity of epsE genes in the exopolysaccharide biosynthesis gene clusters, Biosci Biotechnol Biochem., 77: 2013-2018.
8. Tangyu et al., 2019, Fermentation of plant-based milk alternatives for improved flavour and nutritional value, Applied Microbiology and Biotechnology, 103:9263-9275.
9. van Kranenburg R. et al., 1999, Functional analysis of glycosyltransferase genes from Lactococcus lactis and other gram-positive cocci: complementation, expression, and diversity, J Bacteriol., 181: 6347-6353.
10. Whittall J. J. et al., 2015, Topology of Streptococcus pneumoniae CpsC, a Polysaccharide co-polymerase and BY-kinase adaptor protein, J Bacteriol 197: 120-127.
11. Kleerebezem et al. (2002) Metabolic engineering of Lactococcus lactis: the impact of genomics and metabolic modelling. J. Biotechnol. 98: 199-213.
12. van Kranenburg et al. (1999) Exopolysaccharide biosynthesis in Lactococcus lactis NIZO B40: functional analysis of the glycosyltransferase genes involved in synthesis of the polysaccharide backbone, J Bacteriol., 181: 338-340.
13. van Kranenburg et al. (2000) Nucleotide sequence analysis of the lactococcal EPS plasmid pNZ4000, Plasmid, 43: 130-136.
14. van Casteren et al. (1998) Characterisation and modification of the exopolysaccharide produced by Lactococcus lactis subsp. cremoris B40, Carbohydrate Polymers, 37: 123-130.
15. Agersoe Y et al. (2018) Antimicrobial susceptibility testing and tentative epidemiological cutoff values for five bacillus species relevant for use as animal feed additives or for plant protection, Appl Environ Microbiol, 84 (19).
16. Salvetti et al., 2018, Comparative genomics of the genus Lactobacillus reveals robust phylogroups that provide the basis for reclassification, Appl Environ Microbiol., 84 (17) doi: 10.1128/AEM.00993.

1. A Lactococcus lactis lactic acid bacterium strain comprising an active eps gene cluster capable of producing exopolysaccharide (EPS), wherein the eps gene cluster is selected from:
(i) a nucleotide sequence as defined in SEQ ID NO.: 1;
(ii) a nucleotide sequence as defined in SEQ ID NO.: 2, or a nucleotide sequence which differs by no more than 1 nucleotide from the nucleotide sequence as defined in SEQ ID NO.: 2;
(iii) a nucleotide sequence as defined in SEQ ID NO.: 3, or a nucleotide sequence which differs by no more than 1 nucleotide from the nucleotide sequence as defined in SEQ ID NO.: 3;
(iv) a nucleotide sequence as defined in SEQ ID NO.: 4, or a nucleotide sequence which differs by no more than 1 nucleotide from the nucleotide sequence as defined in SEQ ID NO.: 4; and
(v) a nucleotide sequence as defined in SEQ ID NO.: 5, or a nucleotide sequence which differs by no more than 5 nucleotides from the nucleotide sequence as defined in SEQ ID NO.: 5,
wherein the Lactococcus lactis lactic acid bacterium strain comprising an active eps gene cluster as defined in SEQ ID NO.: 1 belongs to the MLST (multilocus sequence typing) group ST76, wherein the MLST analysis is performed as described in Example 4.

2. The lactic acid bacterium strain of claim 1, wherein the lactic acid bacterium strain is a strain which
(i) generates fermented milks having a shear stress of 50 Pa or more, measured at shear rate 300 s⁻¹, measured under following conditions:
200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s-1, wherein the inoculation temperature is 30° C.; and/or
(ii) generates fermented milks having a shear stress of 24 Pa or more, measured at shear rate 300 s⁻¹, measured under following conditions:
200 ml of soy milk supplemented with 2% glucose (as described in Example 2) are inoculated with 2 ml of an overnight culture of the lactic acid bacterium strain, and left at inoculation temperature until pH^{˜}4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C.

3. The lactic acid bacterium strain of claim 1 (i), wherein the lactic acid bacterium strain is a strain which generates fermented milks having a shear stress of 80 Pa or more measured at shear rate 300 s⁻¹ in the presence of the lactic acid bacterium strain Lactococcus lactis subsp. cremoris DSM 25485, or a mutant or variant therefrom, wherein the shear stress is measured under following conditions:
200 ml semi-fat milk (1.5% fat) is heated to 90° C. for 20 min, followed by cooling to inoculation temperature, and inoculated with 2 ml of an overnight culture of the lactic acid bacterium strains, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C.

4. The lactic acid bacterium strain of any of the preceding claims, wherein the lactic acid bacterium is selected from:
(i) Strain DSM 33204, or a mutant or variant therefrom;
Strain DSM 33205, or a mutant or variant therefrom;
Strain DSM 33220, or a mutant or variant therefrom;
Strain DSM 33221, or a mutant or variant therefrom;
Strain DSM 33218, or a mutant or variant therefrom;
Strain DSM 33219, or a mutant or variant therefrom;
Strain DSM 33224, or a mutant or variant therefrom;
Strain DSM 33197, or a mutant or variant therefrom;
Strain DSM 33196, or a mutant or variant therefrom;
Strain DSM 33195, or a mutant or variant therefrom;
Strain DSM 33194, or a mutant or variant therefrom;
Strain DSM 33226, or a mutant or variant therefrom;
Strain DSM 33223, or a mutant or variant therefrom;
Strain DSM 33193, or a mutant or variant therefrom; and
Strain DSM 33192, or a mutant or variant therefrom;
(ii) Strain DSM 33200, or a mutant or variant therefrom;
Strain DSM 33201, or a mutant or variant therefrom;
Strain DSM 33202, or a mutant or variant therefrom; and
Strain DSM 33203, or a mutant or variant therefrom;
(iii) Strain DSM 33222, or a mutant or variant therefrom.
(iv) Strain DSM 33225, or a mutant or variant therefrom.
(v) Strain DSM 33133, or a mutant or variant therefrom.

5. A composition comprising at least one Lactococcus lactis lactic acid bacterium strain of any one of claims 1-4 and a further lactic acid bacterium strain wherein the further lactic acid bacterium strain is able to (i) generate fermented milks with a pH of about 4.55 in 15 h or less, preferably in 12 h or less, and is able to (ii) generate fermented milks having a shear stress of 40 Pa or more measured at shear rate 300 s⁻¹, measured under following conditions:
200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 ((i), time to pH 4.55) followed by storage at 4° C. until shear stress is measured, typically from 1-7 days, such as for 5 days, followed by gently stirring and measuring the shear stress at shear rate 300 s⁻¹ ((ii), shear stress), wherein the inoculation temperature is 30° C.

6. The composition according to claim 5, wherein the composition comprises at least one Lactococcus lactis lactic acid bacterium strain of any one of claims 1-4 and (a) a lactic acid bacterium strain Lactococcus lactis comprising an active eps gene cluster capable of producing exopolysaccharide (EPS), wherein the eps gene cluster comprises the nucleotide sequences (a), (b) and (c) (a to c4) as defined in (vi), or (b) a lactic acid bacterium strain Lactococcus lactis comprising an active eps gene cluster capable of producing exopolysaccharide (EPS), wherein the eps gene cluster is as defined in (vii):
(vi) (a): a nucleotide sequence encoding a polypeptide having polymerase activity and having at least 95% identity with the amino acid sequence encoded by nucleotides 6955-8145 of SEQ ID NO: 1 (herein termed wzy);
(b): a nucleotide sequence encoding a polypeptide having polysaccharide transporter activity and having at least 95% identity with the amino acid sequence encoded by nucleotides 9309-10727 of SEQ ID NO: 1 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 4008-4478 of SEQ ID NO: 1 (herein termed GT1);
(c2): a nucleotide sequence having at least 95% identity with the amino acid sequence encoded by nucleotides 4478-4960 of SEQ ID NO: 1 (herein termed GT2);
(c3): a nucleotide sequence having at least 95% identity with the amino acid sequence encoded by nucleotides 5015-5965 of SEQ ID NO: 1 (herein termed GT3); and
(c4): a nucleotide sequence having at least 95% identity with the amino acid sequence encoded by nucleotides 6026-6955 of SEQ ID NO: 1 (herein termed GT4);
(vii) a nucleotide sequence as defined in SEQ ID NO.: 5.

7. The composition according to any one of claims 5-6, wherein the composition comprises the Lactococcus lactis lactic acid bacterium strain of any one of claims 1-4 and:
(i) the lactic acid bacterium strain Lactococcus lactis subsp. cremoris DSM 25485, or a mutant or variant therefrom; and/or
(ii) the lactic acid bacterium strain Lactococcus lactis subsp. lactis DSM 33192, or a mutant or variant therefrom; and/or
(iii) the lactic acid bacterium strain Lactococcus lactis DSM 33133, or a mutant or variant therefrom.

8. Use of the lactic acid bacterium strain as defined in any one of claims 1-4 or the composition as defined in any one of claims 5-7, for increasing the viscosity of a fermented milk product.

9. A method of producing a food product comprising at least one stage in which at least one lactic acid bacterium strain as defined in any one of claims 1-4 and/or the composition as defined in any one of claims 5-7 is used.

10. The method according to claim 9, wherein the food product is a dairy product and the method comprises fermenting a milk substrate with at least one lactic acid bacterium strain as defined in any of claims 1-4 and/or with the composition as defined in any one of claims 5-7.

11. A food product comprising at least one lactic acid bacterium strain as defined in any of claims 1-4 and/or the composition as defined in any one of claims 5-7.

12. Use of the Lactococcus lactis subsp. cremoris strain DSM 25485 and/or the Lactococcus lactis subsp. lactis strain DSM 33192 for increasing viscosity of a fermented milk product.

13. The use according to claim 12, wherein the fermented milk product has a shear stress of 50 Pa or more, preferably 55 Pa or more, even more preferably 80 Pa or more, such as 80 Pa, 95 Pa or 98 Pa, measured at shear rate 300 s⁻¹, measured under following conditions:
200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C.

14. The use according to any one of claims 12-13, wherein (i) the Lactococcus lactis subsp. cremoris strain DSM 25485 and/or (ii) Lactococcus lactis subsp. lactis strain DSM 33192 is(are) used in combination with at least one of the Lactococcus lactis lactic acid bacterium strain as defined in any one of claims 1-4.

15. The use according to claim 8, wherein the fermented milk product has a shear stress of 24 Pa or more, such as about 35 Pa, 36 Pa, 45 Pa, 47 Pa, 54 Pa, 56 Pa, 57 Pa, 60 Pa, 62 Pa, 63 Pa, 64 Pa, 71 Pa, 74 Pa, 75 Pa, 79 Pa, 86 Pa, 88 Pa, 93 Pa, 96 Pa, 99 Pa, 102 Pa, 106 Pa or more, measured at shear rate 300 s⁻¹, under the following conditions:
200 ml of soy milk supplemented with 2% glucose (as described in Example 2) are inoculated with 2 ml of an overnight culture of the lactic acid bacterium strain, and left at inoculation temperature until pH^{˜}4.55 (such as pH 4.49, 4.53, 4.54, 4.55 or 4.66), followed by storage at 4° C. until shear stress is measured, typically from 1-7 days, such as for 5 days, followed by gently stirring and measuring the shear stress at shear rate 300 s⁻¹, wherein the inoculation temperature is 30° C.

16. Lactococcus lactis lactic acid bacterium strain Lactococcus lactis subsp. lactis DSM 33192.

17. A method for manufacturing a Lactococcus lactis lactic acid bacterium strain which comprises the following steps:
(a) providing a Lactococcus lactis lactic acid bacterium strain comprising an active eps gene cluster capable of producing exopolysaccharide (EPS), wherein the eps gene cluster is selected from:
(i) a nucleotide sequence as defined in SEQ ID NO.: 1;
(ii) a nucleotide sequence as defined in SEQ ID NO.: 2, or a nucleotide sequence which differs by no more than 1 nucleotide from the nucleotide sequence as defined in SEQ ID NO.: 2;
(iii) a nucleotide sequence as defined in SEQ ID NO.: 3, or a nucleotide sequence which differs by no more than 1 nucleotide from the nucleotide sequence as defined in SEQ ID NO.: 3;
(iv) a nucleotide sequence as defined in SEQ ID NO.: 4, or a nucleotide sequence which differs by no more than 1 nucleotide from the nucleotide sequence as defined in SEQ ID NO.: 4; and
(v) a nucleotide sequence as defined in SEQ ID NO.: 5, or a nucleotide sequence which differs by no more than 5 nucleotides from the nucleotide sequence as defined in SEQ ID NO.: 5;
(b) screening for a Lactococcus lactis lactic acid bacterium strain which is
(i) able to generate fermented milks having a shear stress of 40 Pa or more, preferably 50 Pa or more, measured at shear rate 300 s⁻¹, measured under following conditions:
200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C.; and/or
(ii) able to generate fermented milks having a shear stress greater than 24 Pa, such as about 35 Pa, 36 Pa, 45 Pa, 47 Pa, 54 Pa, 56 Pa, 57 Pa, 60 Pa, 62 Pa, 63 Pa, 64 Pa, 71 Pa, 74 Pa, 75 Pa, 79 Pa, 86 Pa, 88 Pa, 93 Pa, 96 Pa, 99 Pa, 102 Pa, 106 Pa or more, measured at shear rate 300 s⁻¹, under the following conditions:
200 ml of soy milk supplemented with 2% glucose (as described in Example 2) are inoculated with 2 ml of an overnight culture of the lactic acid bacterium strain, and left at inoculation temperature until pH^{˜}4.55 (such as pH 4.49, 4.53, 4.54, 4.55 or 4.66), followed by storage at 4° C. until shear stress is measured, typically from 1-7 days, such as for 5 days, followed by gently stirring and measuring the shear stress at shear rate 300 s⁻¹, wherein the inoculation temperature is 30° C.
